SEQUENCE LISTING (20 sequences)

SEQ ID NO: 1 - DNA, 2016 bp, Phoenix dactylifera
cgccattgacatcgatggaggagctggctgaggaactattgagcttaaattccctgaggtgggggcaagaagcttcagctcgtttgatccggaagtcttgtcgctgattgtgaggaccttatcctctccgcctttggtctcagaaagtggtttgtgggtgttgggatcaatgcctctctgccttagcttcttcttgatgcaggagttccacaggttcttaatctcgttgtctgtcctcccaggcagctgagctgcaatttgagaccatctgaaggggaggaaattgagatacatcagctcatatggattgaattctaagagatgcaatgttgataattgagggaagatgagctgaaggactggttagtacctgttgcccaaggccgcatggagttcgatgatgaggttctcctcctgctgtgaaaaagagcctctcttgagatcaggcctcagataatttgtccacctcaatctgcagctctttccacacctttgaagacctggaatgtgtgattttatcttagaatttggagatactttcatgcagaaatgaactcttttcaaaatattatcccacaaaaggaaagagaagtcggtatgattaattaaaagaattgctctttgctatcttgatttttttttcggatgcattcttgttttaggaggtgatgaggcgaagtggtagacatacctgctagcttgggcacagagctccagcacccatggccatacttagtgatgtgcttgaggagcttctcatcctcctccggggaccagaggcctttccttagcttctgcttgtagcagcaggagtgtctccccattttgggcgagataaccacaatgccaccgaggccttcaaatctccagagaatttggtattggatcctttcagctcgagggggtagcttttgtgagtacgaggattgggtgttggtctctaggaaatgaagtggcaacggagaggaggaattgaattaggaggagaaggccaagcttggagagagagagagagatgtgaaggtgacaatcgactgggtgtgattgggtttgaggttttttaatttcttttctccatttatttccttctttttgaggtggggataggggattgaatattgatcatcttacaatgtaggtctcatcaggctctgagataaatgtttgctcctataatggtggggtatctgccctcgtaaattaatggagaagcagtagcacgatttggtccccatgacttgaaagctaaggcagcaaaaaagtaacattatttgaaagctaacctaacaaaatatagcatgcatgggaaaacactgccttccaagcaatccaatacggaaacaaccgcagatttttggagaagggctaaaaagtcctctcctagcaatagacaaacattaaataataagcaccggcctatacagcagcagtatacctttatcccatttgtgaaggagcgaggcccaaaggaaagtcctaacgcccattcgagttcctaagataatcacgtgtcagagaaaactgggcccattttgctttttccactgatccccaaatccatgatcacgttggagaggctcatcattgattggaaagaacgaaatacctccatacatcttataaaacggagggcacctttctctggtttggaaagagacaactcagacaactgtgtcccttggcggataacttggtatttaccaattgcagaggctggacaaccatatcatggcttctctgaagaaactgaaagaaagtacctggggagcaagtctgcttgaccgaaaaatagactgcttgaagcgtcaagcagttaaaagtacgttaacttagcatcagtctcgcgatatatgctctaataacttttcaccctgtacttttctcttatgaatgcatgttttctttgtgttctttttattttgatgaaaaggtggtcagaggccacaaattttattaaagaatgaaagaattacaatacaattaaggtagtgtggagctaggacagatggaggagga

SEQ ID NO: 2 - DNA, 2021 bp, Phoenix dactylifera
aaaccctgtataataacttctatttagcttcttgagtaagttcctggattgttacaaatagtattaaagcggaattggcccatgacctatgtgtgactatagggtacacatgagtcctttttgggctgattgtgggccgattgtggtgtttgtaattagatttagttggatttagactcttagcctagagtgaactccagtgcttaaatgagaggaggtgaggactcgtgtgggtgtgtgcttagtctcatattggttgtgcagtaggaagatcttgggtacctagatagggccaatgaatctaaataataacttttggctagttttttgggtgaggttctaatttgttacaccagggacaattcaaaatatttatcgagtattaagagtttgccaaaatgatatggagctcatgtatggaaaatttactaggatcatatttggagtttagggtgcagtatcaaaggatggtaagattctttgaacagctgccattgctgcctatgttctatcatgcattttgttatttaatatcagtgcaaaatggatagagtggaaatatgcctttgaagtttaagaaatcaccaagtaccattgtaactcatgctagatttttttttcaatggctgtcaatttaccagtttttctgagagagtataagttttgggagggattcaggtgatttcaagtatgtcaggagaagggtggatgtaatggaggtacatctttgttgtggtgtgatttttgaatatcattggaactgataataagctttttaaggcagtcaaaatatgagaacttctgaatgaatacaaattttaggtatttacagtctgtattgatgtttgaggaacaaaccaaaaagagatcttcatggaagttgttggcagagaagtgatttgaagacatattagagccgaatgcaaacaagtgttggggtagagtttgttaaactgacaatcagctaggcgtgcacgcgcgcgcgcgcgcacacacacatagagagagagagagagagagaaatttgatcttcatgttcatatttgttgcattgaggcaaggagcatgcctttgttattgttagtgttgaaccatgcgaaaacataagacattgcaaaccttggtgtatacaagggacaaactaatcagccaccttgcctaatgttctcatgagtagaatggaaagtaatagaaattcacttgttgtctctagcatgtgtttagcatggttggataagatcaattgaaagaatggctgcctaggtagtggaacatatagctcattgttgcagtcccatttcaacagtttttacatgcttgaggaaatcgagggaattgatggatgtgctaggtggcttagatgaggatagtaagggcgaatggtgagatagaatagggtaaagagataatgaacttctaggacctgtatatatgccaagcagaggttatgttttcttgtttggtataacatgtcaaaacttcaggtgtcactatgtgatggtgtacagcttgcaagggatagtcggcttgggcaaggcacgtactaaaaatacatactccaagagaacttgaagaactatagttatcgttttgagtaaggcctaggatttgaaccttggacctccttaagggcaagcctttagggctcaagtgccaaccgtcatgtttaccaatcacgcatctaatatataatatataaactatttaaatgtaggctttgcttgcacagagttctccatttgctacataacgttattaataaagctatttattgtagataatgagtagggattttatttaatacatttttttatcaaaatataacaatataatatgatcaataagaagtttattagcttattaaatcatgttaaataagaaacttttcattagtactaccaaattaatacataaattgacgatattattaatagtcatatttacataatgtatgctaatattgatgaaaaagattgttcatcagtatcgtaccttacccatatgctgcacaagtcattaga

SEQ ID NO: 3 - DNA, 1979 bp, Phoenix dactylifera
  misc_feature (1)..(1): n is a, c, g, or t
ncgataatactatcgtcctattaataatatattatacggtcatgaacattgaaggacttgtgtgacaaaatcatatttcattacacaaagcagtcataagaacttcatcacatcgaagtttgtgaagatgttctggtgatgaataaataccatgttatttatacttgaatttacaatcaacaattgattggatttaggacacaattcccaacatcaacatgctagtgttttggcttttaggtgttcgaatttcaaattccaggcatccaggcatttgaggcttagaatgaagagttttgatgttctcattccttggctctgatcaagtttttgacatatatatgctctcacaccactttctaccatccataccgaatctgaaattttgtgccctcaaaattgcccacattcttgtgaaatagtctgacaaaattctagtactcgaatgctaacttaggtatccaaaccttcaaacactagtcatttgacatctaatatgatattcaaattttcaattttggtgcactcagagtttttcatgatttcatgggatgatgtaatggcattcaagttcttggatgaaagaggttggatgtttagatgtttagatgctagggttttgacagcaaggccttcgtatctcatatgcttcgattgcactaatttttaaaatttgaacagagaatagagcttatctaatcccctaaagcctctataaatagcctacaaatagccttccactcctcccctcttgggagtaaagaggcaaatggttagtcactctccaatactccattcctattctttttagactcttgggagactctcaaaggatcgtgctacaatgccaagagggctgaaccataaagaaggcctattttctattggtgggtcaagtgaggtacatgggctttatctttatctaagctagactctataggccttcactctctctctctctctctctctctctctctcttcttttcaatttctcttcatatgtatatcttattcacacctcccatgcaagcattagaataggacatttcgtctacctataatattttttaaaaagggacaaagtgtgtcttatgttctattcctcctccaccatcccttatgcctttgtagtctaatattggatcaacttcttttcttttgcaaccatttcctcaaagatctctgtgctgagcatgctttgactcaaggagaaataacccacatcgatgactactgcaattgacatatgcatcttgacaatcaacaattgactggcaatcacacattttagtagagtaaggccttgcactagaatcggagtatcatataagacccaactttccatcaagcacaaagggctacataccaatctctatagatgccatggcaatcatggtggcacactcgaaatttgctcatgggaatagttataccattctaaactttcacatctttctcgcgtaagcatatatatgtagcttagtaacttttatttgtctatttaatttggggttaagcttgggttatatctagattgagaccaaaaagggaggactcatgaaaataggcaatattagtgtaaccatctctcaagttggataatattagtgtgactatcccaagtttcctctagtctcaactagatgaacccatacccttcttcctagatttactaactttgtaagcacccaccctactcctaaggaccctattgaaagtgggatatactctaaaagctctctgcatatttaatatactcgagaaattatgtctatctcctcttatagtcatgtttaatataagatatatgtcatacattgactataaagccagaaaagagactatgagcattatagggataaatccaagtggatgggtgttatccgtaaggaggattggatagatctacatatttgaaactcacttataactaatgggtgagattaggcattatacctataaggttgccgaggtagaga

SEQ ID NO: 4 - DNA, 22 bp, Artificial Sequence
  primer
attgggtgttggtctctaggaa

SEQ ID NO: 5 - DNA, 22 bp, Artificial Sequence
  primer
tcgtgctactgcttctccatta

SEQ ID NO: 6 - DNA, 20 bp, Artificial Sequence
  primer
catggaagttgttggcagag

SEQ ID NO: 7 - DNA, 19 bp, Artificial Sequence
  primer
catgctccttgcctcaatg

SEQ ID NO: 8 - DNA, 20 bp, Artificial Sequence
  primer
tcgtgctacaatgccaagag

SEQ ID NO: 9 - DNA, 20 bp, Artificial Sequence
  primer
ctaatgcttgcatgggaggt

SEQ ID NO: 10 - DNA, 17 bp, Artificial Sequence
  primer
tggatttccattgtgag

SEQ ID NO: 11 - DNA, 17 bp, Artificial Sequence
  primer
cccgaagagacgctatt

SEQ ID NO: 12 - DNA, 20 bp, Artificial Sequence
  primer
gcaggtggactgcaaaatct

SEQ ID NO: 13 - DNA, 20 bp, Artificial Sequence
  primer
ctattggggtgctgatccat

SEQ ID NO: 14 - DNA, 20 bp, Artificial Sequence
  primer
ggagcatacagtgggtttgc

SEQ ID NO: 15 - DNA, 20 bp, Artificial Sequence
  primer
cagcctgggaatgaggatag

SEQ ID NO: 16 - DNA, 20 bp, Artificial Sequence
  primer
gagagatgcgtcagggaatc

SEQ ID NO: 17 - DNA, 20 bp, Artificial Sequence
  primer
ccagaatcttccaagcaagc

SEQ ID NO: 18 - DNA, 18 bp, Phoenix dactylifera
aagtttgaggggctgaga

SEQ ID NO: 19 - DNA, 20 bp, Artificial Sequence
  primer
ttctcagcccctcaaacttc

SEQ ID NO: 20 - DNA, 22 bp, Artificial Sequence
  primer
ggttgcagccatgagctcaacc

(19) United States
(12) Patent Application Publication
(10) Pub. No.: US 2015/0167104 A1
(43) Pub. Date: Jun. 18, 2015

(54) MOLECULAR MARKERS AND METHODS FOR EARLY SEX DETERMINATION IN DATE PALMS

(71) Applicants: INSTITUT DE RECHERCHE POUR LE DEVELOPMENT (IRD), Marseille 2 (FR); LABORATOIRE DE GENETIQUE MOLECULAIRE, Tunis (TN)

(72) Inventors: Frederic Aberlenc, Castelnau-le-Lez (FR); Emira Cherif, Ariana (TN)

(21) Appl. No.: 14/415,071

(22) PCT Filed: Jul. 18, 2013
(86) PCT No.: PCT/EP2013/065168

§ 371 Date: Jan. 15, 2015

(30) Foreign Application Priority Data
Jul. 19, 2012 (FR) 1256998

(51) Int. Cl. C12Q 1/68

(52) U.S. Cl. CPC C12Q 1/6895; C12Q 2600/13; C12Q 2600/156

(57) ABSTRACT

The present invention relates to molecular markers and to the use thereof for distinguishing male date palm plants from female date palm plants. The invention also relates to a method for identifying the sex of date palm plants using these molecular markers, and to a kit for carrying out the method.

### RELATED PATENT APPLICATION

The present application claims priority to French Patent Application number FR 12 56998 filed on Jul. 19, 2012. The content of the French Patent Application is incorporated herein by reference in its entirety.

### BACKGROUND OF THE INVENTION

The date palm (Phoenix dactylifera L.) is a monocotyledonous plant of the Arecaceae family which is widely cultivated for its fruits: the dates. In arid regions, the date palm plays not only a major economic role, through the production of dates which constitute the basis of human and animal diets, but also an ecological role since it gives the oasis its structure. The date palm has been cultivated for thousands of years; the first written works and drawings confirming its cultivation go back 6000 years BC. Date palm is also one of the first plants to have been artificially pollinated (Zohary et al., Science, 1975, 187: 319-327).

In the Maghreb and in the Middle East, the date palm is today mainly propagated asexually, which allows the characteristics of cultivars to be maintained but results in an erosion of the genetic inheritance. Sexual reproduction, used in the Sahel and the Indus Valley, enables the production of several hundreds of seeds, genetic mixing and the creation of varieties that are resistant to biotic and abiotic stresses. The creation of resistant varieties is all the more important that the date palm is currently threatened by several diseases, including vascular fusariosis, called “Bayoud disease”, which is rife in the Maghreb countries and which has led to the loss of close to ten million date palms and of certain varieties of very high quality since the beginning of the century. The climatic changes, which have been harsh in desert regions, in particular in the Mediterranean, also cause considerable hydric stresses to which the current varieties are not adapted.

The creation of resistant varieties is, however, limited by the actual characteristics of the date palm. Indeed, the date palm is a dioic plant, i.e. a plant of which the unisex male (plant with stamens) and female (plant with pistil) flowers are borne by different plants. Therefore, the sexual reproduction of the date palm gives a progeny comprising approximately 50% of male plants (non-productive) and 50% of female plants (date producers). As it happens, it is necessary to wait 6 to 8 years for the induction of the first flowering in order to know the gender of the plants.

In order to solve this problem, the identification of molecular markers allowing early determination of the sex of the date palm has been sought for decades (Bekheet and Hanafy, Date Palm Technology, 2011, 551-566). Zaher et al. (In Acts of the Second International Congress on Biochemistry, Agadir, Morocco, 2006, p. 69-72) identified molecular markers of RAPD (Random Amplified Polymorphism DNA) type, but when these markers were tested in male and female individuals separately, the analysis of the individual RAPD profiles showed a random distribution of the presence of the “marker” band in the two sexes. Recently, certain regions of the date palm genome have been described as sex-linked (Al Dous et al., Nature Biotech., 2011, 29: 521-528). Sex markers, in particular of SNP (Single Nucleotide Polymorphism) type have been identified in these regions (Al Mahmoud et al., Am. J. Bot., 2012, 99: 7-10) but they have been validated on a very small sample and only allow the sex of the date palm to be identified with a 90% certainty. Elmeer et al. (“Marker-assisted sex differentiation in date palm using simple sequence repeats”, 3 Biotech, published online in March 2012) have developed sex markers of SSR (single sequence repeat) type, but these markers, validated on a small sample size, distinguish the males only at 75%.

It is therefore important to develop new strategies for unambiguously selecting the female plants at a young age, and therefore to limit the plantation costs associated with the cultivation of the non-productive male plants. The early sex determination would also open up new perspectives for multiplying by seed the date palm genotypes, reintroducing biodiversity into palm groves and implementing genetic improvement programmes.

### SUMMARY OF THE INVENTION

The present invention generally relates to molecular markers specific of the sex of the date palm. In particular, the invention relates to markers for distinguishing, with 100% certainty, the female plants which produce dates from the male plants which only produce pollen. The markers have been validated on a selection of samples of more than 100 male and female individuals that were representative of the worldwide diversity. The inventive molecular markers also make it possible to distinguish the male individuals in species related to Phoenix dactylifera L., suggesting the existence of a dioic ancestry common to all the species of the genus which appears to date back approximately 60 million years. The identification of these markers by the inventors has been particularly complex due to the presence of duplications in the sex-linked regions of the date palm genome. Exact measurement of the allele sizes for one and the same marker has made it possible to distinguish the alleles specific of male individuals and to dispense with the problem posed by sequence duplications. The markers of the invention, which are of microsatellite or SSR (single sequence repeat) nature, make it possible to reveal a sequence length polymorphism between the genomes of male individuals and female individuals.

Consequently, in a first aspect, the present invention relates to a sex-specific microsatellite marker of date palm, wherein the sex-specific microsatellite marker is chosen from:
the P80 microsatellite marker which, in the 5′→3′ direction, has a (ga) motif and is flanked, in 5′, by the nucleotide sequence occupying positions 73550 to 74549 in the PDK_{—}30s6550963 scaffold (GenBank accession number: GL739764.1), and in 3′, by the nucleotide sequence occupying positions 74566 to 75565 in the PDK_{—}30s6550963 scaffold;
the P50 microsatellite marker which, in the 5′→3′ direction, has an (ag) motif and is flanked, in 5′, by the nucleotide sequence occupying positions 4871 to 5870 in the PDK_{—}30s1202771 scaffold (GenBank accession number: GL744456.1), and in 3′, by the nucleotide sequence occupying positions 5891 to 6889 in the PDK_{—}30s1202771 scaffold; and
the P52 microsatellite marker which, in the 5′→3′ direction, has a (ct) motif and is flanked, in 5′, by the nucleotide sequence occupying positions 9276 to 10275 in the PDK_{—}30s680001 scaffold (GenBank accession number: GL745189.1), and in 3′, by the nucleotide sequence occupying positions 10308 to 11307 in the PDK_{—}30s680001 scaffold.

The invention also relates to the use of a sex-specific microsatellite marker as described above to identify the sex of a date palm.

The invention also relates to a method for identifying the sex of a date palm by detecting, using a microsatellite marker, an SSR polymorphism in the date palm genome.

In certain preferred embodiments, the microsatellite marker is a sex-specific microsatellite marker of date palm according to the invention and is chosen from the markers P80, P50 and P52.

In certain embodiments, the method is characterized in that the detection of the SSR polymorphism comprises:
amplification of a portion of genomic DNA of the date palm tested, using a pair of primers specific of the microsatellite marker, in order to obtain amplicons; and
analysis of the amplicons obtained in order to determine the sex of the date palm tested.

In certain embodiments, the step of amplifying a portion of the genomic DNA of the date palm tested is carried out using a pair of primers consisting of:
a forward SSR primer comprising, or consisting of, between 15 and 50 consecutive nucleotides of the sequence of at most 1000 nucleotides flanking, in 5′, position 74550 in the PDK_{—}30s6550963 scaffold, and a reverse SSR primer comprising, or consisting of, between 15 and 50 consecutive nucleotides of the sequence complementary to the sequence of at most 1000 nucleotides flanking, in 3′, position 74565 in the PDK_{—}30s6550963 scaffold; or
a forward SSR primer comprising, or consisting of, between 15 and 50 consecutive nucleotides of the sequence of at most 1000 nucleotides flanking, in 5′, position 5871 in the PDK_{—}30s1202771 scaffold, and a reverse SSR primer comprising, or consisting of, between 15 and 50 consecutive nucleotides of the sequence complementary to the sequence of at most 1000 nucleotides flanking, in 3′, position 5890 in the PDK_{—}30s1202771 scaffold; or
a forward SSR primer comprising, or consisting of, between 15 and 50 consecutive nucleotides of the sequence of at most 1000 nucleotides flanking, in 5′, position 10276 in the PDK_{—}30s680001 scaffold, and a reverse SSR primer comprising, or consisting of, between 20 and 35 consecutive nucleotides of the sequence complementary to the sequence of at most 1000 nucleotides flanking, in 3′, position 10307 in the PDK_{—}30s680001 scaffold.

In certain preferred embodiments, the amplification of a portion of the date palm genomic DNA is carried out using a pair of primers consisting of:
a forward SSR primer of sequence SEQ ID NO: 4 and a reverse SSR primer of sequence SEQ ID NO: 5; or
a forward SSR primer of sequence SEQ ID NO: 6 and a reverse SSR primer of sequence SEQ ID NO: 7; or
a forward SSR primer of sequence SEQ ID NO: 8 and a reverse SSR primer of sequence SEQ ID NO: 9.

In certain embodiments, analyzing the amplicons obtained for determining the sex of the date palm tested comprises:
separating the amplicons obtained by amplification of a portion of the genomic DNA of the date palm tested according to their size, and
comparing the sizes of said amplicons with the sizes of the amplicons obtained by amplification, under the same conditions, of a portion of the genomic DNA of a control date palm.

The separation of the amplicons according to their size may be carried out by any appropriate technique, for instance by agarose gel electrophoresis or polyacrylamide gel electrophoresis.

The control date palm is generally a date palm of known gender and known origin. Generally, several controls are used in the comparison (for example, the controls may include at least one male eastern date palm, at least one male western date palm, at least one female eastern date palm and at least one female western date palm).

When the amplification is carried out using a pair of primers consisting of a forward SSR primer of sequence SEQ ID NO: 4 and a reverse SSR primer of sequence SEQ ID NO: 5, the presence of an amplicon containing 194 nucleotides and/or of an amplicon containing 310 nucleotides indicates that the date palm tested is a male date palm plant.

When the amplification is carried out with a pair of primers comprising a forward SSR primer of sequence SEQ ID NO: 6 and a reverse SSR primer of sequence SEQ ID NO: 7, the presence of an amplicon containing 180, 182, 214, 223, 225 or 227 nucleotides indicates that the date palm tested is a male date palm plant.

When the amplification is carried out with a pair of primers comprising a forward SSR primer of sequence SEQ ID NO: 8 and a reverse SSR primer of sequence SEQ ID NO: 9, the presence of an amplicon containing 188, 190, 191, 193, 197 or 199 nucleotides indicates that the date palm tested is a male date palm plant.

In certain embodiments, analyzing the amplicons obtained for determining the sex of the date palm tested comprises:
sequencing the separated amplicons.

In certain embodiments, analyzing the amplicons obtained for determining the sex of the date palm tested comprises:
separating the amplicons obtained by amplification of a portion of the genomic DNA of the date palm tested according to their size; and
sequencing the separated amplicons.

In certain embodiments, a method for identifying the sex of the date palm according to the invention is carried out on genomic DNA extracted from a sample of the date palm. The sample of the date palm tested may be a sample of protoplast, of callus, of embryos, of leaf, of trunk, of root, of offshoots, of cutting of the date palm or any combination thereof.

In certain embodiments, a method according to the invention is used for the early identification of the sex of the date palm, and the date palm sample is collected from a date palm plant up to 6 to 8 years after sowing.

The invention also relates to pairs of primers specific of the microsatellite markers of the invention.

In certain embodiments, the pair of primers consists of a forward SSR primer comprising, or consisting of, between 15 and 50 consecutive nucleotides of the sequence of at most 1000 nucleotides flanking, in 5′, position 74550 in the PDK_{—}30s6550963 scaffold, and a reverse SSR primer comprising, or consisting of, between 15 and 50 consecutive nucleotides of the sequence complementary to the sequence of at most 1000 nucleotides flanking, in 3′, position 74565 in the PDK_{—}30s6550963 scaffold.

For example, such a pair of primers may consist of a forward primer of sequence SEQ ID NO: 4 and a reverse primer of sequence SEQ ID NO: 5.

In other embodiments, the pair of primers consists of: a forward SSR primer comprising, or consisting of, between 15 and 50 consecutive nucleotides of the sequence of at most 1000 nucleotides flanking, in 5′, position 5871 in the PDK_{—}30s1202771 scaffold, and a reverse SSR primer comprising, or consisting of, between 15 and 50 consecutive nucleotides of the sequence complementary to the sequence of at most 1000 nucleotides flanking, in 3′, position 5890 in the PDK_{—}30s1202771 scaffold.

For example, such a pair of primers may consist of: a forward primer of sequence SEQ ID NO: 6 and a reverse primer of sequence SEQ ID NO: 7.

In yet another embodiment, the pair of primers consists of: a forward SSR primer comprising, or consisting of, between 15 and 50 consecutive nucleotides of the sequence of at most 1000 nucleotides flanking, in 5′, position 10276 in the PDK_{—}30s680001 scaffold, and a reverse SSR primer comprising, or consisting of, between 15 and 50 consecutive nucleotides of the sequence complementary to the sequence of at most 1000 nucleotides flanking, in 3′, position 10307 in the PDK_{—}30s680001 scaffold.

For example, such a pair of primers may consist of: a forward primer of sequence SEQ ID NO: 8 and a reverse primer of sequence SEQ ID NO: 9.

In certain embodiments, at least one of the SSR primers making up a pair of primers according to the invention comprises a detectable label.

The invention also relates to the use of a pair of SSR primers according to the invention for identifying the sex of a date palm.

The invention further provides a kit for identifying the sex of a date palm, comprising at least one pair of SSR primers according to the invention and instructions for carrying out a method according to the invention.

In another aspect, the present invention provides a male-specific marker of date palm, wherein in the 5′→3′ direction, the male-specific marker is located at position 74489 in scaffold PDK_{—}30s6550963 and has the sequence set forth in SEQ ID NO: 18.

The invention also relates to the use of the male-specific marker to identify the sex of a date palm.

The invention also relates to a method for identifying the sex of a date palm comprising steps of:
amplifying a portion of genomic DNA of the date palm tested using a pair of primers specific for a male-specific marker in order to obtain amplicons, and
detecting if amplicons are produced, wherein detection of amplicons indicates that the date palm tested as a male date palm,
   wherein, in the 5′→3′ direction, the male-specific marker is located at position 74489 in scaffold PDK_{—}30s6550963 and has the sequence set forth in SEQ ID NO: 18.

In certain embodiments, the pair of primers specific for the male-specific marker consists of:
a forward PCR primer comprising, or consisting of, between 15 and 18 consecutive nucleotides of the sequence of at most 2000 nucleotides flanking, in 5′, position 74489 in the PDK_{—}30s6550963 scaffold, and
a reverse PCR primer comprising, or consisting of, between 15 and 18 consecutive nucleotides of the sequence complementary to the sequence of at most 2000 nucleotides flanking, in 3′, position 74506 in the PDK_{—}30s6550963 scaffold.

In certain embodiments, the pair of primers specific for the male-specific marker consists of a forward PCR primer comprising, or consisting of, the sequence set forth in SEQ ID NO: 19 and a reverse PCR primer comprising, or consisting of, the sequence set forth in SEQ ID NO: 20.

In certain embodiments, the method is carried out on genomic DNA extracted from a sample of the date palm. The sample of the date palm tested may be a sample of protoplast, of callus, of embryos, of leaf, of trunk, of root, of offshoots, of cutting of the date palm or any combination thereof.

In certain embodiments, a method according to the invention is used for the early identification of the sex of the date palm, and the date palm sample is collected from a date palm plant up to 6 to 8 years after sowing.

The present invention also provides PCR primer pairs for the identification of the sex of a date palm, wherein the PCR primer pairs are specific of the male-specific marker. In certain preferred embodiment, a PCR primer pair according to the invention consists of:
a forward PCR primer comprising, or consisting of, between 15 and 18 consecutive nucleotides of the sequence of at most 2000 nucleotides flanking, in 5′, position 74489 in the PDK_{—}30s6550963 scaffold, and
a reverse PCR primer comprising, or consisting of, between 15 and 18 consecutive nucleotides of the sequence complementary to the sequence of at most 2000 nucleotides flanking, in 3′, position 74506 in the PDK_{—}30s6550963 scaffold.

In certain embodiments, the pair of primers specific for the male-specific marker consists of a forward PCR primer comprising, or consisting of, the sequence set forth in SEQ ID NO: 19 and a reverse PCR primer comprising, or consisting of, the sequence set forth in SEQ ID NO: 20.

In certain embodiments, at least one of the PCR primers making up a pair of primers according to the invention comprises a detectable label.

The invention also relates to the use of a pair of PCR primers according to the invention for identifying the sex of a date palm.

The invention further provides a kit for identifying the sex of a date palm, comprising at least one pair of PCR primers according to the invention and instructions for carrying out a method according to the invention.

These and other objects, advantages and features of the present invention will become apparent to those of ordinary skill in the art having read the following detailed description of the preferred embodiments.

### BRIEF DESCRIPTION OF THE DRAWING

FIG. 1: Correspondence analysis graphs showing the genetic differentiations generated by the autosomal SSRs (A) and the sex-specific SSRs (B). Each point corresponds to an individual of the subgroup. In graph (A), the individuals are randomly distributed without any grouping together. In graph (B), the individuals are clearly grouped together according to the sexual phenotype.

FIG. 2: Allele distribution of the P80 (A), P50 (B), and P52 (C) loci. The left part of the figure corresponds to the male genotypes and the right part to the female genotypes. Each point represents an allele. The alleles common to the male and female individuals (X) are represented in white and the male-specific alleles (Y) are represented in black. The female individuals only have alleles which are common to the males and to the females, whereas the male individuals have alleles that are common to both sexes and alleles that are male-specific. The western and eastern male genotypes have, respectively, one or two male-specific alleles. In this example, the allele sizes presented comprise the 19 nucleotides of the M13 tail added to the forward sequences for detecting the amplicons.

FIG. 3: Allelic distribution of P50, P52 and P80 loci in different species of the Phoenix genus. The left part of the figure corresponds to the male genotypes and the right part to the female genotypes. Each point represents an allele. X alleles, shared by males and females are in white, male specific Y alleles are in black.

FIG. 4: Validation of the male-specific marker on 10 males and 10 females of the P. dactylifera species. The male-specific sequence (SEQ ID NO: 18) used as forward primer allows the amplification of a band only in male individuals, not in females.

FIG. 5: Validation of the male-specific marker on 6 species of the Phoenix genus. The male-specific sequence (SEQ ID NO: 18) used as forward primer allows the amplification of a band only in male individuals, not in females for the species P. canariensis, P. atlantica, P. reclinata, P. sylvestris, P. roebellinii and P. rupicola. Under the conditions used, no specific band was detected for P. loureiroi.

### DETAILED DESCRIPTION OF CERTAIN PREFERRED EMBODIMENTS

As mentioned above, the present invention relates to molecular markers that are specific of the sex of date palm plants and to the use of these molecular markers for distinguishing between male plants and female plants. The invention has the advantage of allowing the early selection of female plants and therefore of limiting the plantation costs associated with the cultivation of the non-productive male plants. Another advantage of the markers of the invention is that they are “universal” in that they can be used regardless of the origin, variety or cultivar of the date palm.

### I—Date Palm Sex-Specific Markers

The present Applicants have identified, in the genome of the date palm, three sex-specific microsatellite (or SSR) sequences. These microsatellite sequences are located in genomic scaffolds which have previously been described as comprising a high number of SNPs (Single Nucleotide Polymorphisms) between the male individuals and the female individuals (Al-Dous et al., 2011—Nature Biotech., 2011, 29: 521-528).

The terms “microsatellite” and “SSR” are used herein interchangeably and refers to a DNA sequence formed by a continuous repeat of motifs composed of 1 to 10 nucleotides and most commonly flanked by conserved regions. Generally, the length of these SSR sequences, i.e. the number of repeats of the motif, is variable from one species to another, from one individual to another and from one allele to another in one and the same individual. The SSR sequences of the invention are sex-specific for the date palm in that the length of these sequences is indicative of the gender of the date palm.

The information provided below and defining each SSR marker of the invention (motif and locus) concerns the 5′→3′ strand of the scaffold where the SSR is located. As will be recognized by those skilled in the art, given that an SSR sequence is a double-stranded DNA sequence, the invention also encompasses the complementary motif and the complementary locus (which are located on the 3′→5′ strand).

The first microsatellite sequence of the invention has a (ga) motif and is located in the PDK_{—}30s6550963 scaffold (GenBank accession number: GL739764.1). The sequence of this scaffold, which has a total length of 95115 nucleotides, was obtained from a female date palm of the Khalas variety. In this scaffold, the (ga) motif is repeated 8 times and occupies positions 74550 to 74565. The sequence SEQ ID NO: 1, which is a portion of the PDK_{—}30s6550963 scaffold, shows the (ga) motif as it is present in this scaffold (see underlined region) and the left and right flanking sequences of 1000 base pairs of the microsatellite. This microsatellite site of sequence SEQ ID NO: 1 has been named P80 by the inventors.

SEQ ID NO: 1
CGCCATTGACATCGATGGAGGAGCTGGCTGAGGAACTATTGAGCTTAAAT
TCCCTGAGGTGGGGGCAAGAAGCTTCAGCTCGTTTGATCCGGAAGTCTTG
TCGCTGATTGTGAGGACCTTATCCTCTCCGCCTTTGGTCTCAGAAAGTGG
TTTGTGGGTGTTGGGATCAATGCCTCTCTGCCTTAGCTTCTTCTTGATGC
AGGAGTTCCACAGGTTCTTAATCTCGTTGTCTGTCCTCCCAGGCAGCTGA
GCTGCAATTTGAGACCATCTGAAGGGGAGGAAATTGAGATACATCAGCTC
ATATGGATTGAATTCTAAGAGATGCAATGTTGATAATTGAGGGAAGATGA
GCTGAAGGACTGGTTAGTACCTGTTGCCCAAGGCCGCATGGAGTTCGATG
ATGAGGTTCTCCTCCTGCTGTGAAAAAGAGCCTCTCTTGAGATCAGGCCT
CAGATAATTTGTCCACCTCAATCTGCAGCTCTTTCCACACCTTTGAAGAC
CTGGAATGTGTGATTTTATCTTAGAATTTGGAGATACTTTCATGCAGAAA
TGAACTCTTTTCAAAATATTATCCCACAAAAGGAAAGAGAAGTCGGTATG
ATTAATTAAAAGAATTGCTCTTTGCTATCTTGATTTTTTTTTCGGATGCA
TTCTTGTTTTAGGAGGTGATGAGGCGAAGTGGTAGACATACCTGCTAGCT
TGGGCACAGAGCTCCAGCACCCATGGCCATACTTAGTGATGTGCTTGAGG
AGCTTCTCATCCTCCTCCGGGGACCAGAGGCCTTTCCTTAGCTTCTGCTT
GTAGCAGCAGGAGTGTCTCCCCATTTTGGGCGAGATAACCACAATGCCAC
CGAGGCCTTCAAATCTCCAGAGAATTTGGTATTGGATCCTTTCAGCTCGA
GGGGGTAGCTTTTGTGAGTACGAGGATTGGGTGTTGGTCTCTAGGAAATG
AAGTGGCAACGGAGAGGAGGAATTGAATTAGGAGGAGAAGGCCAAGCTTG
GAGAGAGAGAGAGAGATGTGAAGGTGACAATCGACTGGGTGTGATTGGGT
TTGAGGTTTTTTAATTTCTTTTCTCCATTTATTTCCTTCTTTTTGAGGTG
GGGATAGGGGATTGAATATTGATCATCTTACAATGTAGGTCTCATCAGGC
TCTGAGATAAATGTTTGCTCCTATAATGGTGGGGTATCTGCCCTCGTAAA
TTAATGGAGAAGCAGTAGCACGATTTGGTCCCCATGACTTGAAAGCTAAG
GCAGCAAAAAAGTAACATTATTTGAAAGCTAACCTAACAAAATATAGCAT
GCATGGGAAAACACTGCCTTCCAAGCAATCCAATACGGAAACAACCGCAG
ATTTTTGGAGAAGGGCTAAAAAGTCCTCTCCTAGCAATAGACAAACATTA
AATAATAAGCACCGGCCTATACAGCAGCAGTATACCTTTATCCCATTTGT
GAAGGAGCGAGGCCCAAAGGAAAGTCCTAACGCCCATTCGAGTTCCTAAG
ATAATCACGTGTCAGAGAAAACTGGGCCCATTTTGCTTTTTCCACTGATC
CCCAAATCCATGATCACGTTGGAGAGGCTCATCATTGATTGGAAAGAACG
AAATACCTCCATACATCTTATAAAACGGAGGGCACCTTTCTCTGGTTTGG
AAAGAGACAACTCAGACAACTGTGTCCCTTGGCGGATAACTTGGTATTTA
CCAATTGCAGAGGCTGGACAACCATATCATGGCTTCTCTGAAGAAACTGA
AAGAAAGTACCTGGGGAGCAAGTCTGCTTGACCGAAAAATAGACTGCTTG
AAGCGTCAAGCAGTTAAAAGTACGTTAACTTAGCATCAGTCTCGCGATAT
ATGCTCTAATAACTTTTCACCCTGTACTTTTCTCTTATGAATGCATGTTT
TCTTTGTGTTCTTTTTATTTTGATGAAAAGGTGGTCAGAGGCCACAAATT
TTATTAAAGAATGAAAGAATTACAATACAATTAAGGTAGTGTGGAGCTAG
GACAGATGGAGGAGGA

The second microsatellite sequence of the invention has an (ag) SSR motif and is located in the PDK_{—}30s1202771 scaffold (GenBank accession number: GL744456.1). The sequence of this scaffold, which has a total length of 20074 nucleotides, was obtained from a female date palm of the Khalas variety. In this scaffold, the (ag) motif is repeated 10 times and occupies positions 5871 to 5890. The sequence SED ID NO: 2, which is a portion of the PDK_{—}30s1202771 scaffold, shows the (ag) motif as it is present in this scaffold (see underlined region) and the left and right flanking sequences of 1000 base pairs of the microsatellite. This microsatellite site has been named P50 by the inventors.

SEQ ID NO: 2
AAACCCTGTATAATAACTTCTATTTAGCTTCTTGAGTAAGTTCCTGGATT
GTTACAAATAGTATTAAAGCGGAATTGGCCCATGACCTATGTGTGACTAT
AGGGTACACATGAGTCCTTTTTGGGCTGATTGTGGGCCGATTGTGGTGTT
TGTAATTAGATTTAGTTGGATTTAGACTCTTAGCCTAGAGTGAACTCCAG
TGCTTAAATGAGAGGAGGTGAGGACTCGTGTGGGTGTGTGCTTAGTCTCA
TATTGGTTGTGCAGTAGGAAGATCTTGGGTACCTAGATAGGGCCAATGAA
TCTAAATAATAACTTTTGGCTAGTTTTTTGGGTGAGGTTCTAATTTGTTA
CACCAGGGACAATTCAAAATATTTATCGAGTATTAAGAGTTTGCCAAAAT
GATATGGAGCTCATGTATGGAAAATTTACTAGGATCATATTTGGAGTTTA
GGGTGCAGTATCAAAGGATGGTAAGATTCTTTGAACAGCTGCCATTGCTG
CCTATGTTCTATCATGCATTTTGTTATTTAATATCAGTGCAAAATGGATA
GAGTGGAAATATGCCTTTGAAGTTTAAGAAATCACCAAGTACCATTGTAA
CTCATGCTAGATTTTTTTTTCAATGGCTGTCAATTTACCAGTTTTTCTGA
GAGAGTATAAGTTTTGGGAGGGATTCAGGTGATTTCAAGTATGTCAGGAG
AAGGGTGGATGTAATGGAGGTACATCTTTGTTGTGGTGTGATTTTTGAAT
ATCATTGGAACTGATAATAAGCTTTTTAAGGCAGTCAAAATATGAGAACT
TCTGAATGAATACAAATTTTAGGTATTTACAGTCTGTATTGATGTTTGAG
GAACAAACCAAAAAGAGATCTTCATGGAAGTTGTTGGCAGAGAAGTGATT
TGAAGACATATTAGAGCCGAATGCAAACAAGTGTTGGGGTAGAGTTTGTT
AAACTGACAATCAGCTAGGCGTGCACGCGCGCGCGCGCGCACACACACAT
AGAGAGAGAGAGAGAGAGAGAAATTTGATCTTCATGTTCATATTTGTTGC
ATTGAGGCAAGGAGCATGCCTTTGTTATTGTTAGTGTTGAACCATGCGAA
AACATAAGACATTGCAAACCTTGGTGTATACAAGGGACAAACTAATCAGC
CACCTTGCCTAATGTTCTCATGAGTAGAATGGAAAGTAATAGAAATTCAC
TTGTTGTCTCTAGCATGTGTTTAGCATGGTTGGATAAGATCAATTGAAAG
AATGGCTGCCTAGGTAGTGGAACATATAGCTCATTGTTGCAGTCCCATTT
CAACAGTTTTTACATGCTTGAGGAAATCGAGGGAATTGATGGATGTGCTA
GGTGGCTTAGATGAGGATAGTAAGGGCGAATGGTGAGATAGAATAGGGTA
AAGAGATAATGAACTTCTAGGACCTGTATATATGCCAAGCAGAGGTTATG
TTTTCTTGTTTGGTATAACATGTCAAAACTTCAGGTGTCACTATGTGATG
GTGTACAGCTTGCAAGGGATAGTCGGCTTGGGCAAGGCACGTACTAAAAA
TACATACTCCAAGAGAACTTGAAGAACTATAGTTATCGTTTTGAGTAAGG
CCTAGGATTTGAACCTTGGACCTCCTTAAGGGCAAGCCTTTAGGGCTCAA
GTGCCAACCGTCATGTTTACCAATCACGCATCTAATATATAATATATAAA
CTATTTAAATGTAGGCTTTGCTTGCACAGAGTTCTCCATTTGCTACATAA
CGTTATTAATAAAGCTATTTATTGTAGATAATGAGTAGGGATTTTATTTA
ATACATTTTTTTATCAAAATATAACAATATAATATGATCAATAAGAAGTT
TATTAGCTTATTAAATCATGTTAAATAAGAAACTTTTCATTAGTACTACC
AAATTAATACATAAATTGACGATATTATTAATAGTCATATTTACATAATG
TATGCTAATATTGATGAAAAAGATTGTTCATCAGTATCGTACCTTACCCA
TATGCTGCACAAGTCATTAGA

The third microsatellite sequence of the invention has a (ct) SSR motif and is located in the PDK_{—}30s680001 scaffold (GenBank accession number: GL745189.1). The sequence of this scaffold, which has a total length of 17202 nucleotides, was obtained from a female date palm of the Khalas variety. In this scaffold, the (ct) motif is repeated 16 times and occupies positions 10276 to 10307. The sequence SED ID NO: 3, which is a portion of the PDK_{—}30s680001 scaffold, shows the (ct) motif as it is present in this scaffold (see underlined region) and the left and right flanking sequences of the microsatellite. This microsatellite site has been named P52 by the inventors.

SEQ ID NO: 3
NCGATAATACTATCGTCCTATTAATAATATATTATACGGTCATGAACATT
GAAGGACTTGTGTGACAAAATCATATTTCATTACACAAAGCAGTCATAAG
AACTTCATCACATCGAAGTTTGTGAAGATGTTCTGGTGATGAATAAATAC
CATGTTATTTATACTTGAATTTACAATCAACAATTGATTGGATTTAGGAC
ACAATTCCCAACATCAACATGCTAGTGTTTTGGCTTTTAGGTGTTCGAAT
TTCAAATTCCAGGCATCCAGGCATTTGAGGCTTAGAATGAAGAGTTTTGA
TGTTCTCATTCCTTGGCTCTGATCAAGTTTTTGACATATATATGCTCTCA
CACCACTTTCTACCATCCATACCGAATCTGAAATTTTGTGCCCTCAAAAT
TGCCCACATTCTTGTGAAATAGTCTGACAAAATTCTAGTACTCGAATGCT
AACTTAGGTATCCAAACCTTCAAACACTAGTCATTTGACATCTAATATGA
TATTCAAATTTTCAATTTTGGTGCACTCAGAGTTTTTCATGATTTCATGG
GATGATGTAATGGCATTCAAGTTCTTGGATGAAAGAGGTTGGATGTTTAG
ATGTTTAGATGCTAGGGTTTTGACAGCAAGGCCTTCGTATCTCATATGCT
TCGATTGCACTAATTTTTAAAATTTGAACAGAGAATAGAGCTTATCTAAT
CCCCTAAAGCCTCTATAAATAGCCTACAAATAGCCTTCCACTCCTCCCCT
CTTGGGAGTAAAGAGGCAAATGGTTAGTCACTCTCCAATACTCCATTCCT
ATTCTTTTTAGACTCTTGGGAGACTCTCAAAGGATCGTGCTACAATGCCA
AGAGGGCTGAACCATAAAGAAGGCCTATTTTCTATTGGTGGGTCAAGTGA
GGTACATGGGCTTTATCTTTATCTAAGCTAGACTCTATAGGCCTTCACTC
TCTCTCTCTCTCTCTCTCTCTCTCTCTCTTCTTTTCAATTTCTCTTCATA
TGTATATCTTATTCACACCTCCCATGCAAGCATTAGAATAGGACATTTCG
TCTACCTATAATATTTTTTAAAAAGGGACAAAGTGTGTCTTATGTTCTAT
TCCTCCTCCACCATCCCTTATGCCTTTGTAGTCTAATATTGGATCAACTT
CTTTTCTTTTGCAACCATTTCCTCAAAGATCTCTGTGCTGAGCATGCTTT
GACTCAAGGAGAAATAACCCACATCGATGACTACTGCAATTGACATATGC
ATCTTGACAATCAACAATTGACTGGCAATCACACATTTTAGTAGAGTAAG
GCCTTGCACTAGAATCGGAGTATCATATAAGACCCAACTTTCCATCAAGC
ACAAAGGGCTACATACCAATCTCTATAGATGCCATGGCAATCATGGTGGC
ACACTCGAAATTTGCTCATGGGAATAGTTATACCATTCTAAACTTTCACA
TCTTTCTCGCGTAAGCATATATATGTAGCTTAGTAACTTTTATTTGTCTA
TTTAATTTGGGGTTAAGCTTGGGTTATATCTAGATTGAGACCAAAAAGGG
AGGACTCATGAAAATAGGCAATATTAGTGTAACCATCTCTCAAGTTGGAT
AATATTAGTGTGACTATCCCAAGTTTCCTCTAGTCTCAACTAGATGAACC
CATACCCTTCTTCCTAGATTTACTAACTTTGTAAGCACCCACCCTACTCC
TAAGGACCCTATTGAAAGTGGGATATACTCTAAAAGCTCTCTGCATATTT
AATATACTCGAGAAATTATGTCTATCTCCTCTTATAGTCATGTTTAATAT
AAGATATATGTCATACATTGACTATAAAGCCAGAAAAGAGACTATGAGCA
TTATAGGGATAAATCCAAGTGGATGGGTGTTATCCGTAAGGAGGATTGGA
TAGATCTACATATTTGAAACTCACTTATAACTAATGGGTGAGATTAGGCA
TTATACCTATAAGGTTGCCGAGGTAGAGA

Consequently, the invention relates to three microsatellite markers that are sex-specific for date palm plants, defined by their motif and their locus, and more specifically here by their motif and the conserved nucleotide sequences which flank the motif in 5′ and in 3′. The terms “nucleotide sequence”, “sequence” and “nucleic sequence” are used herein interchangeably. These terms are intended to denote a succession of nucleotides defining a region of a nucleic acid molecule, and which may be either under the form a single strain or double strain DNAs or under the form of transcription products thereof.

In particular, the invention relates to a sex-specific microsatellite marker of the date palm, named P80, characterized in that, in the 5′→3′ direction, the microsatellite marker has a (ga) motif and is flanked, in 5′, by the nucleotide sequence occupying positions 73550 to 74549 in the PDK_{—}30s6550963 scaffold, and in 3′, by the nucleotide sequence occupying positions 74566 to 75565 in the PDK_{—}30s6550963 scaffold.

The invention also relates to a sex-specific microsatellite marker of the date palm, named P50, characterized in that, in the 5′→3′ direction, the SSR marker has an (ag) motif and is flanked, in 5′, by the nucleotide sequence occupying positions 4871 to 5870 in the PDK_{—}30s1202771 scaffold, and in 3′, by the nucleotide sequence occupying positions 5890 to 6689 in the PDK_{—}30s1202771 scaffold.

The invention also relates to a sex-specific microsatellite marker of the date palm, named P52, characterized in that, in the 5′→3′ direction, the SSR marker has a (ct) motif and is flanked, in 5′, by the nucleotide sequence occupying positions 9276 to 10275 in the PDK_{—}30s680001 scaffold, and in 3′, by the nucleotide sequence occupying positions 10308 to 11307 in the PDK_{—}30s680001 scaffold.

The present Applicants have also identified other microsatellite sequences in regions of the date palm genome, identified as being sex-linked by Al Dous et al. (Nature Biotech., 2011, 29: 521-528). These microsatellite sequences, which form an integral part of the present invention, are described in Example 2 below. It is obvious that, using the data provided in Example 2, those skilled in the art can determine whether or not these microsatellite sequences are specific of the gender of date palm plants.

### II—Use of the Date Palm Sex-Specific Markers for Distinguishing Male and Female Date Palm Plants

As demonstrated by the present Applicants, the molecular markers described above can be used for identifying the sex of date palm plants (regardless of their origin or variety). Generally, a method of sex determination according to the invention is carried out by detecting, using a microsatellite marker, an SSR polymorphism in the genome of the date palm, and comprises the amplification of a region of the genome of the date palm to be tested, the region comprising a microsatellite sequence according to the invention.

### Date Palm Samples

In a method according to the invention, the step of amplifying a region (or portion) of the genome of the date palm comprising a repeated SSR motif according to the invention is carried out on a sample of the date palm to be tested, and preferably on genomic DNA extracted from the sample of the date palm.

Any date palm sample containing genomic DNA can therefore be used in a method of the present invention. For example, the genomic DNA can be extracted from protoplasts, from calluses, from embryos, from leaves, from trunks, from roots, from offshoots or from cuttings of date palms. It is obvious that, when the method is used for identifying the sex of a date palm at a young age, the genomic DNA will preferably be extracted from leaves taken from a small seedling resulting from the germination of a seed. In the context of the present invention, the term “young age” is used to describe a date palm in which the first flowerings have not yet taken place. Generally, the young age corresponds to the first 6 to 8 years after sowing.

Methods for extracting DNA from biological tissues are well known in the art (see, for example, Sambrook et al., “Molecular Cloning—A Laboratory Manual”, Cold Spring Harbor Laboratory Press, 1989). There are also many commercially available kits (for example, from BD Biosciences Clontech (Palo Alto, Calif.), Epicenter Technologies (Madison, Wis.), Gentra Systems, Inc. (Minneapolis, Minn.), MicroProbe Corp. (Bothell, Wash.), Organon Teknika (Durham, N.C.) and Qiagen Inc. (Valencia, Calif.)) which can be used for extracting the DNA from date palm samples.

### Microsatellite Sequence Amplification

As will be recognized by those skilled in the art, in the implementation of the invention, the amplification of a portion of the date palm genome comprising a microsatellite sequence according to the invention can be carried out by any appropriate technique known in the art, since the technique is not a limiting factor of the invention.

Preferably, in a method according to the invention, the genomic DNA amplification reactions are carried out by PCR (Polymerase Chain Reaction) amplification (Mullis and Faloona, Methods Enzymol., 1987, 155: 355-350), which offers the advantage of analyzing the molecular markers in a short period of time while using low DNA concentrations. The flanking regions of the microsatellites serve as primers during the PCR. Given that these regions are conserved, a pair of primers specific for these flanking regions specifically only amplifies this microsatellite (and not another).

The various amplicons (or amplification products) generated from a given microsatellite region have characteristic and reproducible sizes. The variation in the sizes of the PCR products is caused by differences in the number of repeats of the motif of the microsatellite. These sizes are therefore indicative of the length (if the date palm sample is homozygous) or of the lengths (if the date palm sample is heterozygous) of the microsatellite sequence in the individual.

Those skilled in the art know how to select the optimum conditions (temperature, duration and number of cycles, pH, and reagent concentrations) for carrying out a PCR amplification (“PCR Protocols: A Guide to Methods and Applications”, M. A. Innis (Ed.), 1990, Academic Press: New York; “PCR Strategies”, M. A. Innis (Ed.), 1995, Academic Press: New York; “Polymerase chain reaction: basic principles and automation in PCR: A Practical Approach”, McPherson et al. (Eds.), 1991, IRL Press: Oxford; R. K. Saiki et al., Nature, 1986, 324: 163-166).

### Primers and Probes for the Amplification of a Portion of the Genome of the Date Palm Comprising an SSR Sequence

Using the nucleotide sequence of a scaffold in which a microsatellite of the invention is located and the position of this microsatellite in the scaffold, those skilled in the art know how to design SSR primers suitable for the amplification of a portion of the genome of the date palm, which comprises the microsatellite sequence.

The terms “primer” and “PCR primer” are used herein interchangeably and denote an oligonucleotide which is capable of acting as a starting point for the synthesis of an amplification product, when it is placed under suitable amplification conditions (for example, salt concentration, temperature and pH) in the presence of nucleotides and of a nucleic acid polymerization agent (for example, a DNA polymerase). A primer according to the invention comprises an oligonucleotide advantageously containing between 5 and 50 nucleotides, generally between 15 and 50 nucleotides, preferably between 20 and 35 nucleotides and even more preferably between 20 and 25 nucleotides (for example, 20, 21, 22, 23, 24 or 25 nucleotides).

The term “SSR primer” refers to a primer which is specific for a flanking or adjacent region of the microsatellite region and which, in combination with another SSR primer, is capable of specifically amplifying the microsatellite region. The amplification is generally carried out using a pair of SSR primers comprising a forward (or “sense”) primer and a reverse (or “antisense”) primer, which each hybridize to one of the two strands of the genomic DNA.

Preferably, a forward SSR primer according to the invention comprises, or consists of, a sequence of 15 to 50 consecutive nucleotides, preferably of 20 to 35 consecutive nucleotides, and even more preferably of 20 to 25 consecutive nucleotides, of the sequence of at most 1000 nucleotides flanking, in 5′, the microsatellite region according to the invention. Preferably, a reverse SSR primer according to the invention comprises, or consists of, a sequence of 15 to 50 consecutive nucleotides, preferably of 20 to 35 consecutive nucleotides, and even more preferably of 20 to 25 consecutive nucleotides, of the sequence complementary to the sequence of at most 1000 nucleotides flanking, in 3′, the microsatellite region according to the invention.

The expression “sequence complementary to” a given nucleotide sequence is intended to mean a sequence which forms, by hybridization, a stable duplex with said nucleotide sequence. The expression “sequence complementary to” denotes both the complementary sequence presented in the 3′→5′ direction and the complementary sequence presented in the 5′→3′ direction (i.e. the reverse complementary sequence). The term “hybridization”, as used herein, refers to the head-to-tail association of two single-stranded polynucleotides by Watson-Crick pairings (A-T, G-C). In certain cases, the hybridization is perfect, i.e. the sequences are totally complementary. Thus, for example, the expression “the sequence complementary to the sequence SEQ ID NO: 1” is the nucleotide sequence which is perfectly or totally complementary to SEQ ID NO: 1.

Thus, in the implementation of a method of the invention, the amplification of a region of the date palm genome, the region comprising the microsatellite sequence having a (ga) motif of the P80 site, is preferably carried out using a pair of SSR primers comprising:
a forward SSR primer comprising, or consisting of, between 15 and 50 consecutive nucleotides of the sequence of at most 1000 nucleotides flanking, in 5′, position 74550 in the PDK_{—}30s6550963 scaffold, and
a reverse SSR primer comprising, or consisting of, between 15 and 50 consecutive nucleotides of the sequence complementary to the sequence of at most 1000 nucleotides flanking, in 3′, position 74565 in the PDK_{—}30s6550963 scaffold.

The sequence of at most 1000 nucleotides may comprise any number of nucleotides preferably between 1000 and 250, for example 1000, 900, 800, 700, 600, 500, 400, 300 or 250, or any number in between.

In one particular embodiment, a pair of primers for the amplification of a region of the date palm genome comprising the microsatellite sequence having a (ga) motif of the P80 site comprises a forward primer comprising, or consisting of, the sequence set forth in SEQ ID NO: 4 (ATTGGGTGTTGGTCTCTAGGAA) and a reverse primer comprising, or consisting of, the sequence set forth in SEQ ID NO: 5 (TCGTGCTACTGCTTCTCCATTA).

The amplification of a region of the genome of the date palm comprising the microsatellite sequence having a (ag) motif of the P50 site is preferably carried out using a pair of SSR primers comprising:
a forward SSR primer comprising, or consisting of, between 15 and 50 consecutive nucleotides of the sequence of at most 1000 nucleotides flanking, in 5′, position 5871 in the PDK_{—}30s1202771 scaffold, and
a reverse SSR primer comprising, or consisting of, between 15 and 50 consecutive nucleotides of the sequence complementary to the sequence of at most 1000 nucleotides flanking, in 3′, position 5890 in the PDK_{—}30s1202771 scaffold.

The sequence of at most 1000 nucleotides may comprise any number of nucleotides preferably between 1000 and 250, for example 1000, 900, 800, 700, 600, 500, 400, 300 or 250, or any number in between.

In one particular embodiment, a pair of primers for the amplification of a region of the date palm genome comprising the microsatellite sequence having a (ag) motif of the P50 site comprises a forward primer comprising, or consisting of, the sequence set forth in SEQ ID NO: 6 (CATGGAAGTTGTTGGCAGAG) and a reverse primer comprising, or consisting of, the sequence set forth in SEQ ID NO: 7 (CATGCTCCTTGCCTCAATG).

The amplification of a region of the date palm genome comprising the microsatellite sequence having a (ct) motif of the P52 site is preferably carried out using a pair of SSR primers comprising:
a forward SSR primer comprising, or consisting of, between 15 and 50 consecutive nucleotides of the sequence of at most 1000 nucleotides flanking, in 5′, position 10276 in the PDK_{—}30s680001 scaffold, and
a reverse SSR primer comprising, or consisting of, between 15 and 50 consecutive nucleotides of the sequence complementary to the sequence of at most 1000 nucleotides flanking, in 3′, position 10307 in the PDK_{—}30s680001 scaffold.

The sequence of at most 1000 nucleotides may comprise any number of nucleotides preferably between 1000 and 250, for example 1000, 900, 800, 700, 600, 500, 400, 300 or 250, or any number in between.

In yet another particular embodiment, a pair of primers for the amplification of a region of the date palm genome comprising the microsatellite sequence having a (ct) motif of the P52 site comprises a forward primer comprising, or consisting of, the sequence set forth in SEQ ID NO: 8 (TCGTGCTACAATGCCAAGAG) and a reverse primer comprising, or consisting of, the sequence set forth in SEQ ID NO: 9 (CTAATGCTTGCATGGGAGGT).

In certain embodiments, a primer according to the invention is labelled so as to allow its detection (and, consequently, detection of the amplification products or amplicons obtained by PCR). Various types of labels, known to those skilled in the art, can be used (radioactive labelling, fluorescence labelling, chemiluminescence labelling, labelling of M13 type, etc.). The label may be integrated into an oligonucleotide comprised in the primer, or associated with the oligonucleotide (for example by covalent bonding). The term “labelled primer” or “probe” is therefore intended to denote a primer which contains, or which is associated with or bonded to (for example covalently), a detectable label.

The primers can be prepared by any suitable method known to those skilled in the art, chosen in particular from the conventional methods for oligonucleotide synthesis, for instance solid-phase synthesis methods. For example, the primers according to the invention may be prepared using an oligonucleotide synthesizer (such as those sold, for example, by Applied Biosystems or GE Healthcare). Likewise, methods for labelling the oligonucleotides are known in the art.

The present invention relates to the primers described herein (or any other primer which can be deduced from the information provided) and also to the use thereof for identifying the sex of date palm plants by amplification of a portion of the date palm genome which comprises a microsatellite sequence according to the invention. In particular, the invention also relates to the primers which can be designed in the flanking sequences of the microsatellite sequences described in Example 2 below.

### Amplicon Analysis

In a method for sex determination in date palms, after amplification of a portion of the date palm genome comprising a microsatellite sequence according to the invention, the amplicons are analyzed in order to determine the sex of the date palm tested.

The analysis of the amplicons can be carried out using any suitable method. It generally comprises separating the amplicons according to their size (and therefore according to the size of the microsatellite sequence). For example, the separation can be carried out by agarose gel or polyacrylamide gel electrophoresis (a technique which allows separation of DNA fragments as a function of their electric charge and of their size). The separation is followed by detection of the separated fragments by staining with ethidium bromide or with silver, or else by detection of the detectable label of the primer (by fluorescence, radioactivity, etc.).

To determine the sex of the date palm tested, the sizes of the amplicons obtained can be compared with the sizes of the amplicons obtained by amplification of the genomic DNA of a control date palm, the amplifications being carried out under the same conditions. The term “control date palm”, as used herein, refers to a date palm of known gender and of known origin.

The step of comparing the sizes of the amplicons obtained from the sample of date palm tested may comprise using several control date palms. Preferably, the control date palms include at least one western male date palm, at least one eastern male date palm, at least one western female date palm and at least one eastern female date palm. As used herein, the term “western date palm” refers to any date palm originating from western crops (for example, Tunisia, Morocco, Italy, Spain, Algeria, Mauritania). As used herein, the term “eastern date palm” refers to any date palm originating from eastern crops (for example, Djibouti, Oman, Iraq, Iran, Syria, Jordan, Israel, Turkey, Lebanon, the Arabian Peninsula, Afghanistan, Pakistan, India).

Alternatively, when a pair of SSR primers specific of a microsatellite site has been tested and validated in a study including a large number of date palms representative of worldwide diversity, the sizes of the amplicons can be compared to the sizes of the amplicons which have been identified, by this study, as being indicative of a male date palm. The “control date palm” is, in this case, a collection of date palms of the same sex and of the same origin.

Thus, for example, the present Applicants have shown that, when the amplification is carried out using a pair of primers comprising a forward primer of sequence SEQ ID NO: 4 and a reverse primer of sequence SEQ ID NO: 5, the presence of an amplicon containing 194 nucleotides and/or of an amplicon containing 310 nucleotides indicates that the date palm tested is a male palm tree. More specifically, the Applicants have shown that, if the fragment amplified has a length of 194 nucleotides, the date palm tested is a western male individual, and if the fragment amplified has a length of 310 nucleotides, the date palm tested is an eastern male individual.

When the amplification is carried out with a pair of primers comprising a forward primer of sequence SEQ ID NO: 6 and a reverse primer of sequence SEQ ID NO: 7, the presence of an amplicon containing 180, 182, 214, 223, 225 or 227 nucleotides indicates that the date palm tested is a male palm tree. More specifically, the Applicants have shown that, if a single fragment is amplified, with a length of 180 nucleotides, the date palm tested is a western male individual. If two fragments are amplified, having a length of 180 nucleotides and of 223, 225 or 227 nucleotides or, if two fragments are amplified, having a length of 182 nucleotides and 214 nucleotides, the date palm is an eastern male individual.

When the amplification is carried out with a pair of primers comprising a forward primer of sequence SEQ ID NO: 8 and a reverse primer of sequence SEQ ID NO: 9, the presence of an amplicon containing 188, 190, 191, 193, 197 or 199 nucleotides indicates that the date palm tested is a male palm tree. More specifically, the Applicants have shown that, if the fragment amplified has a length of 191 or 193 nucleotides, the date palm tested is a western male individual, and if the fragment amplified has a length of 188, 190, 197 or 199 nucleotides, the date palm tested is an eastern male individual.

In order to determine the sex of the date palm tested, after separation according to their size, the amplicons can be sequenced in order to determine the number(s) of repeats of the SSR motif in the genome of the date palm tested. The methods for sequencing DNA fragments are known in the art. In particular, it is possible to use automatic sequencers such as those available from Beckman, Applied BioSystems, or LiCor Biosciences.

### III—Date Palm Male-Specific Marker

The present Applicants have also identified, in a region close to P80, an 18 nucleotide long sequence which is only present in the genome of male date palm individuals (see Example 4 below) and which is-male specific not only in P. dactylifera but also in other species of the Phoenix genus, such as P. canariensis, P. sylvestris, P. roebellenii, P. atlantica, P. reclinata and P. rupicola.

In the 5′→3′ direction, this male-specific sequence is located at position 74489 in scaffold PDK_{—}30s6550963 (GenBank accession number: GL739764.1) and has the sequence set forth in SEQ ID NO: 18 (AAGTTTGAGGGGCTGAGA). This sequence is flanked, in 5′, by the nucleotide sequence occupying positions 73489 to 74488 in the PDK_{—}30s6550963 scaffold, and in 3′, by the nucleotide sequence occupying positions 74507 to 75506 in the PDK_{—}30s6550963 scaffold.

This male-specific sequence is a marker that can be used for identifying the sex of date palm plants (regardless of their origin or variety). Generally, a method of sex determination using this molecular marker comprises the amplification of a region of the genome of the date palm to be tested, the region comprising the male-specific sequence according to the invention.

As indicated above for the sex-specific microsatellite molecular markers, the amplification of a portion of the date palm genome comprising the male-specific sequence can be carried out using any suitable technique known to the skilled person, such as PCR amplification. As known in the art, the regions flanking the male-specific sequence can be used to develop PCR primer pairs, each primer pair containing a forward PCR primer and a reverse PCR primer.

Thus, in the implementation of a method of the invention, the amplification of a portion of the date palm genome comprising the male-specific sequence, is preferably carried out using a pair of PCR primers comprising:
a forward PCR primer comprising, or consisting of, between 15 and 18 consecutive nucleotides, preferably of 20 to 35 consecutive nucleotides, and even more preferably of 20 to 25 consecutive nucleotides, of the sequence of at most 2000 nucleotides flanking, in 5′, position 74489 in the PDK_{—}30s6550963 scaffold, and
a reverse PCR primer comprising, or consisting of, between 15 and 18 consecutive nucleotides preferably of 20 to 35 consecutive nucleotides, and even more preferably of 20 to 25 consecutive nucleotides, of the sequence complementary to the sequence of at most 2000 nucleotides flanking, in 3′, position 74506 in the PDK_{—}30s6550963 scaffold.

In certain embodiments, at least one primer of a primer pair is labelled, as described above.

The present invention encompasses the primers described above, and any other primer and primer pair that can be deduced from the information provided herein, as well as the use thereof for identifying the gender of date palm plants by amplification of a portion of the date palm genome which comprises the male-specific sequence.

After PCR amplification, the amplification mixture may be migrated on a gel. In such a method of the invention, the presence of amplicons is indicative of a male date palm individual, while the absence of amplicons is indicative of a female date palm individual.

### IV—Kits for Date Palm Sex Determination

The present invention is also directed to kits comprising material useful for date palm sex determination according to a method of the invention. In particular, the present invention relates to kits for date palm sex determination, containing material allowing the detection, in a date palm genome, of the polymorphism of at least one microsatellite sequence according to the invention. One of the advantages of the kits of the invention is that they can be used throughout the world (i.e. regardless of the origin or the cultivar of the date palm to be tested).

In general, a kit according to the invention comprises at least one pair of SSR primers described herein allowing the amplification of a date palm sex marker according to the invention. Alternatively or additionally, a kit according to the invention may comprise one pair of PCR primers described herein allowing the amplification of the male-specific marker of the invention. A kit according to the invention can be designed so as to be used with a particular amplification technique, in particular a PCR technique.

A kit according to the invention may also comprise reagents or solutions for extracting genomic DNA from date palm samples, PCR amplification reagents or solutions, reagents or solutions for separating amplicons as a function of their size, sequencing reagents or solutions, and/or detection means. Protocols for using these reagents and/or solutions may be included in the kit.

The various components of the kit may be provided in solid form (for example in lyophilized form) or in liquid form. A kit may optionally comprise a container for each of the reagents or solutions, and/or containers for carrying out certain steps of the method of the invention.

A kit according to the invention may also comprise instructions for carrying out the method of the invention for detecting, in the genome of date palm plants, the SSR polymorphism of at least one date palm sex marker according to the invention and/or instructions for detecting, in the genome of date palm plants, the presence or absence of the male-specific marker of the invention. The instructions for carrying out a method according to the invention may comprise instructions for extracting genomic DNA from date palm samples, instructions regarding the PCR amplification conditions, instructions regarding the analysis of the amplicons obtained, and/or instructions for interpreting the results obtained.

A kit according to the invention may also comprise a note in the form stipulated by a government agency regulating the preparation, sale and use of biological products.

Unless otherwise defined, all the technical and scientific terms used herein have the same meaning as generally understood in the field. All the publications, patent applications, patents and other references mentioned herein are each incorporated herein by reference in its entirety.

### EXAMPLES

The following examples describe some of the preferred modes of making and practicing the present invention. However, it should be understood that the examples are for illustrative purposes only and are not meant to limit the scope of the invention. Furthermore, unless the description in an Example is presented in the past tense, the text, like the rest of the specification, is not intended to suggest that experiments were actually performed or data were actually obtained.

### Example 1

### Identification and Validation of Date Palm Sex-Specific Microsatellite Molecular Markers

### Materials and Methods

Date Palm Samples.

The characteristics of the date palm samples used in this study are represented in Table 1 below.

TABLE 1
Distribution of the date palm samples according to their origin and gender.

| | Number of samples | |
|---|---|---|
| Origin | Male | Female |
| Tunisia | 22 | 19 |
| Morocco | 0 | 1 |
| Italy | 2 | 2 |
| Djibouti | 20 | 20 |
| Oman | 4 | 6 |
| Syria | 1 | 2 |
| Iraq | 3 | 2 |

DNA Extraction.

Each sample is composed of leaves which were lyophilized for 72 hours using the Alpha1-4LD Plus lyophilizer (Fisher Scientific, France). The lyophilized leaves were ground using the TissueLyser System (Qiagen, USA), and then the extraction was carried out using the Dneasy plant kit (Qiagen, USA) according to the manufacturer's protocol.

The DNA obtained was assayed with the Tecan GENios™ spectrofluorimeter (Tecan, Switzerland). The concentrations of all the samples were adjusted to 10 ng/μl for the rest of the manipulation.

Microsatellite Amplification by PCR.

The PCR reactions were carried out using an Eppendorf thermocycler (AG, Germany). A reaction volume of 20 μl was used, containing 10 ng of genomic DNA, 10× reaction buffer, 2 mM MgCl₂, 200 μM dNTPs, 0.5 U of polymerase, 0.4 pmol of “sense” (or forward) primer 5′-extended with an M13 sequence, 2 pmol of “antisense” primer, and 2 pmol of an M13 sequence labelled with a fluorochrome, and MilliQ water.

The touchdown PCR amplifications were carried out using the following parameters: denaturation for 2 minutes at 94° C., followed by 6 cycles at 94° C. for 45 seconds, 60° C. for 1 minute and 72° C. for 1 minute, then 30 cycles at 94° C. for 45 seconds, 55° C. for 1 minute and 72° C. for 1.5 minutes, then 10 cycles at 94° C. for 45 seconds, 53° C. for 1 minute, 72° C. for 1.5 minutes, and a final step of extension at 72° C. for 10 minutes.

The microsatellite amplicons were analyzed using the ABI 3130XL genetic analyser (Applied BioSystems, USA).

The allele sizes were identified using the GeneMapper software v3.7 (Applied BioSystems, Foster city, CA, USA). In this analysis, only the sites generating sex-specific alleles were retained.

In Silico Search.

An in silico search of the SSR sequences was carried out in the 24 scaffolds of the sequence of the date palm genome that were identified by Al-Dous et al. (Nature Biotech., 2011, 29: 521-528) as containing a high number of SNPs. This in silico search was carried out using the “SSR_pipeline-v2.pl” script (Poncet et al., 2006, Mol. Genet. Genomics, 2006, 276: 436-449), which integrates three free softwares: Tandem Repeat Finder (Benson, Nucleic Acids Research, 1999, 27: 573-580), Primer3 (Rosen and Skaletzy, 2000 Methods in Molecular Biology. Humana Press, Totowa, N.J., pp. 365-386) and BLAST (Altschul et al., J Mol Biol. 1990, 215: 403-10). The Primer3 software, which is part of the Websat program, was used for designing the primers specific of the flanking regions of the potential SSR markers.

Determination of the Genetic Diversification Index R_{ST} and of the Genetic Variation Linked to Sex-Specific Microsatellites and to Autosomal Microsatellites.

The genetic differentiation within the selection of samples was estimated by calculating the R_{ST} index according to the stepwise mutation model (Slatkin, Genetics, 1995, 139: 457-462), with the GenAlEx 6.41 program (Peakall and Smouse, Mol. Ecol. Notes, 2006, 6: 288-295) by means of an AMOVA, and the significance of the R_{ST} was verified via 10000 permutations. This index is considered to be better suited to the analysis of microsatellite markers since it is often more appropriate for biological information (Slatkin, Genetics, 1995, 139: 457-462). Factorial correspondence analysis FCA (Genetix) was used for the graphic representation of the genetic structure generated by the microsatellite markers.

### Results

The inventors first performed an in silico search of the SSR (single sequence repeat) sequences in the 24 scaffolds of the sequence of the date palm genome which were identified by Al-Dous et al. (Nature Biotech., 2011, 29: 521-528) as containing a high number of SNPs (single nucleotide polymorphisms) between both sexes. They analyzed 34 microsatellites. Three of them were demonstrated to be potentially sex-linked. These three microsatellites are P80, P50 and P52 described in the present document.

Then, given the high complexity of the date palm (i.e. the length of the lifecycle), the crop practices which favor the females, and the lack of genetic distinctions in segregation, the inventors opted for a population approach on a large geographic scale, similar to genetic association studies.

In order to demonstrate the specificity of the P80, P50 and P52 markers, four random microsatellites of the date palm genome were selected. These four microsatellites are mPdCIR078 located in the AJ571685 sequence (GenBank accession number: AJ571685.1), mPdIRD031 located in the PDK_{—}30s801751 scaffold (GenBank accession number: GL741806.1), mPdIRD033 located in the PDK_{—}30s712151 scaffold (GenBank accession number: GL739681.1), and mPdIRD040 located in the PDK_{—}30s862741 scaffold (GenBank accession number: GL740192.1). The characteristics of all the SSRs studied are given in Table 2 below.

TABLE 2
Characteristics of the microsatellites studied.

| | SSR | | Tm |
|---|---|---|---|
| SSR site | motif | Sequences of primers used | (° C.) |
| P80 | (ga) | SEQ ID NO: 4 and | 60 |
| | | SEQ ID No.: 5 | |
| P50 | (ag) | SEQ ID NO: 6 and | 60 |
| | | SEQ ID No.: 7 | |
| P52 | (ct) | SEQ ID NO: 8 and | 60 |
| | | SEQ ID No.: 9 | |
| mPdCIR078 | (ga) | SEQ ID NO.: 10 = | 50 |
| | | TGGATTTCCATTGTGAG (f) | |
| | | SEQ ID NO: 11 = | |
| | | CCCGAAGAGACGCTATT (r) | |
| mPdIRD031 | (cca) | SEQ ID NO: 12 = | 60 |
| | | GCAGGTGGACTGCAAAATCT (f) | |
| | | SEQ ID NO: 13 = | |
| | | CTATTGGGGTGCTGATCCAT (r) | |
| mPdIRD033 | (cag) | SEQ ID NO: 14 = | 60 |
| | | GGAGCATACAGTGGGTTTGC (f) | |
| | | SEQ ID NO: 15 = | |
| | | CAGCCTGGGAATGAGGATAG (r) | |
| mPdIRD040 | (ccagtg) | SEQ ID NO: 16 = | 60 |
| | | GAGAGATGCGTCAGGGAATC (f) | |
| | | SEQ ID NO: 17 = | |
| | | CCAGAATCTTCCAAGCAAGC (r) | |

(f) = forward primer,
(r) = reverse primer

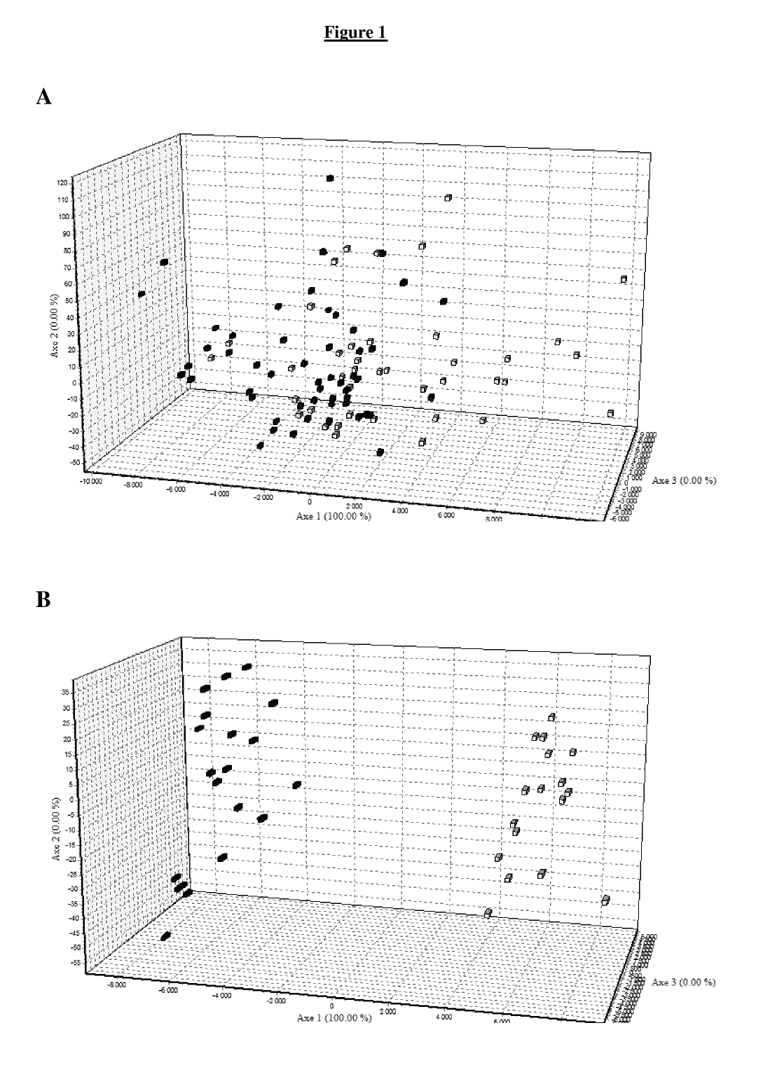

More than a hundred date palm samples (52 males and 55 females) were obtained from various traditional western regions (Tunisia, Morocco and Italy) and eastern regions (Djibouti, Oman, Iraq and Syria). The R_{ST} index (Slatkin, Genetics, 1995, 139: 457-462) measured for each of the three candidate microsatellites demonstrated that these sites exhibit significantly high levels of genetic differentiation (see Table 3); and the factorial correspondence analysis (FCA) identified a clearly sex-dependent population structure and demonstrated two subgroups—one male, the other female (FIG. 1A). Conversely, the four microsatellites randomly chosen in the date palm genome (Billote et al., Molecular Ecology Notes, 2004, 4: 256-258) gave R_{ST} values which were low and non-significant and a homogeneous male/female distribution without any sex-based grouping of individuals (FIG. 1B), which is in agreement with an autosomal localization of these microsatellites. These results confirm that the P80, P50 and P52 microsatellites are linked to the sexual phenotype.

TABLE 3
Characteristics of the microsatellites studied

| SSR site | R_{ST} value for both sexes¹ |
|---|---|
| P80 | 0.171 |
| P50 | 0.182 |
| P52 | 0.461 |
| mPdCIR078 | −0.007 |
| mPdIRD031 | −0.009 |
| mPdIRD033 | −0.006 |
| mPdIRD040 | −0.003 |

¹GENALEX 6.1

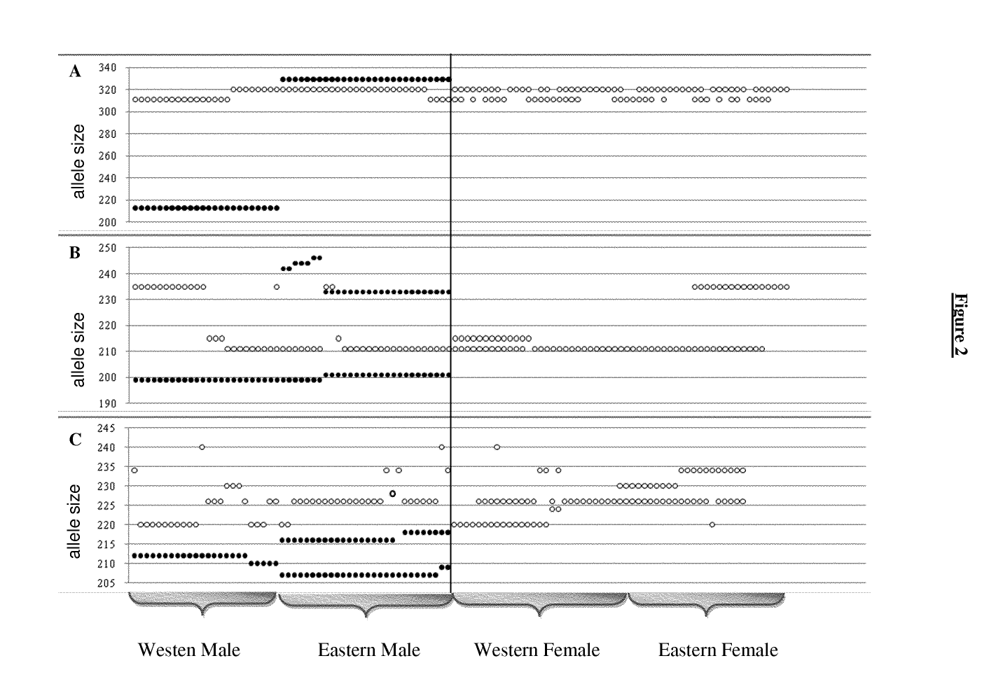

The allele frequencies and the allele size distributions between the male and female subgroups for each of the three sex-specific sites were compared in order to identify the sex-specific alleles. P80 generated four alleles, two of which (P80_{—}292 and P80_{—}301) are common to the males and females, and two of which (P80_{—}194 and P80_{—}310) (FIG. 2A) are male-specific. P50 was found to have essentially the same allele distribution as P80, with two strictly male alleles (P50_{—}180 and P50_{—}182). However, P50 also has a surprising characteristic, where the eastern male individuals have four alleles (P50_{—}214, P50_{—}223, P50_{—}225 and P50_{—}227), all being male-specific (FIG. 2B). The supernumerary alleles may result from a duplication of this site in the male genome. No duplication of this site was observed in the female individuals. The same allele distribution according to sex was observed with P52, which generates four male-specific alleles (P52_{—}188, P52_{—}190, P52_{—}191 and P52_{—}193), with two alleles duplicated in the eastern males (P52_{—}197 and P52_{—}199) (FIG. 2C). It is important to note that, for these three microsatellite sites, the sum of the frequencies of the male alleles in the male subgroup was exactly 0.5 and all the males were heterozygous. In comparison, no significant deviation from the Hardy-Weinberg model (Fᵢₛ not different from 0) was observed in the females, or for the four random SSRs, whether in the males or the females (results not shown).

The inventors therefore developed a set of molecular markers that are sex-specific for the date palm. These markers were 100% validated on a selection of samples of date palm genotypes that are representative of a wide geographic diversity. They constitute a reliable tool which will shorten the time required to select the female plants and will facilitate genetic improvement of the species.

Moreover, preliminary tests revealed that the P80, P50 and P52 markers which allow sex-determination in the Phoenix dactylifera date palm also allow to distinguish the male individuals from the female individuals in several other species of the Phoenix genus, in particular P. canariensis, P. sylvestris, P. roebelenii, P. atlatica and P. reclinata, (see Example 3).

### Example 2

### Identification of Date Palm Sex-Specific Microsatellite Markers in Regions of the Date Palm Genome Identified as being Sex-Linked

### Materials and Methods

In Silico Search.

An in silico search of the SSR sequences was carried out in the 24 scaffolds of the sequence of the date palm genome which were identified by Al-Dous et al. (Nature Biotech., 2011, 29: 521-528) as containing a high number of SNPs. This in silico search was carried out using the “SSR_pipeline-v2.pl” script (Poncet et al., 2006, Mol. Genet. Genomics, 2006, 276: 436-449), which integrates three free softwares: Tandem Repeat Finder (Benson, Nucleic Acids Research, 1999, 27: 573-580), Primer3 (Rosen and Skaletzy, 2000 Methods in Molecular Biology. Humana Press, Totowa, N.J., pp. 365-386) and BLAST (Altschul et al., J Mol Biol. 1990, 215: 403-10).

### Results

The SSRs identified in the PDK_{—}30s695331 scaffold (GenBank accession number: ACYX02059972-17590 nucleotides in length) and their respective locations in this scaffold are given in the following table:

| SSR | Positions occupées dans le scaffold PDK_30s695331 |
|---|---|
| A₄ | (221-224), (473-476), (511-514), (557-560), (692-695), (1059-1062), (1453-1456), |
| | (2181-2184), (2225-2228), (2426-2429), (2754-2757), (2842-2845), (2868-2871), |
| | (3143-3146), (3290-3293), (3497-3500), (3701-3704), (3838-3841), (3979-3982), |
| | (3992-3995), (4293-4296), (5134-5137), (5376-5379), (5404-5407), (7843-7846), |
| | (8325-8328), (8459-8462), (9213-9216), (9223-9226), (9635-9638), (11177-11180), |
| | (11498-11501), (12394-12397), (12448-12451), (12474-12477), (12603-12606), |
| | (12926-12929), (12981-12984), (12986-12989), (13061-13064), (13222-13225), |
| | (13841-13844), (14854-14857), (14919-14922), (15019-15022), (15489-15492), |
| | (15733-15736), (16198-16201), (16217-16220), (16223-16226), (16724-16727), |
| | (16758-16761), (16776-16779) |
| T₄ | (460-463), (1276-1279), (1359-1362), (1425-1428),(1773-1776), (1907-1910), (1992- |
| | 1995), (2315-2318), (2402-2405), (2407-2410), (3083-3086), (3206-3209), (3520- |
| | 3523), (3612-3615), (4211-4214), (4960-4963), (5064-5067), (5125-5128), (5213- |
| | 5216), (5225-5228), (5239-5242), (5286-5289), 5978-5981), (5995-5998, (6101- |
| | 6104), (6167-6170), (6303-6306), 5846-5849), (5637-5640), (5658-5661), (5716- |
| | 5719), (6441-6444), (6468-6471), (6498-6501), (6612-6615), (6720-6723), (6750- |
| | 6753), (6896-6899), (7692-7695), (7788-7791), (8529-8532), (8537-8540), (8641- |
| | 8644), (8857-8860), (9841-9844), (10386-10389), (10647-10650), (11161-11164), |
| | (11181-11184), (11222-11225), (11837-11840), (12079-12082), (12497-12500), |
| | (12623-12626), (12815-12818), (13014-13017), (13242-13245), (13251-13254), |
| | (13510-13513), (14044-14047), (14472-14475), (14513-14516), (14680-14683), |
| | (15465-15468), (16132-16135), (16455-16458), (17155-17158), (17186-17189) |
| G₄ | (507-510), (553-556), (2067-2070), (4935-4938), (5796-5799), (7174-7177), (7254- |
| | 7257), (7451-7454), (7502-7505), (8364-8367), (8699-8702), (8944-8947), (9121- |
| | 9124), (9134-9137), (9649-9652), (10678-10681), (11439-11442), (11697-11700), |
| | (11857-11860), (12141-12144), (12685-12688), (12714-12717), (12749-12752), |
| | (12875-12878), (13199-13202), (13851-13854), (14382-14385), (14413-14416), |
| | (15443-15446), (16062-16065) |
| C₄ | (186-189), (515-518), (561-564), (595-598), (725-728), (1115-1118), (1167-1170), |
| | (1184-1187), (1245-1258), (1260-1263), (1391-1394), (1421-1424), (1605-1608), |
| | (4710-4713), (4822-4825), (4873-4876), (5169-5172), (5753-5756), (7755-7758), |
| | (8277-8280), (7985-7988), (8130-8133), (9333-9336), (9714-9717), (9780-9783), |
| | (10026-10029), (10817-10820), (12758-12761), (13339-13342), (15422-15425), |
| | (15645-15648), (15699-15702) |
| A₅ | (502-506), (907-911), (1704-1708), (2449-2453), (3461-3465), (3474-3478), (3484- |
| | 3488), (3652-3656), (3900-3904), (4094-4098), (4966-4970), (5850-5854), (6365- |
| | 6369), (6379-6383), (8290-8294), (8369-8373), (8964-8968), (8986-8990), (11022- |
| | 11026), (11799-11803), (14263-14267), (15685-15689), (15981-15985) |
| T₅ | (321-325), (1699-1703), (1964-1968), (2210-2214), (2303-2307), (2334-2338), (2684- |
| | 2688), (3214-3218), (3679-3683), (4069-4073), (4257-4261), (4991-4995), (5356- |
| | 5360), (5466-5470), (5550-5554), (5607-5611), (5815-5819), (5869-5873), (6859- |
| | 6863), (7660-7664), (11807-11811), (13582-13586), (14430-14434), (14591-14595), |
| | (15493-15497), (16316-16320), (16433-16437) |
| G₅ | (6405-6409), (8737-8741), (9767-9771), (17003-17007), (17275-17279), (17417- |
| | 17421) |
| C₅ | (528-532), (1221-1225), (1325-1329), (9907-9911), (10290-10294), (12791-12795) |
| A₆ | (900-905), (3374-3379), (3658-3663), (6543-6548), (7399-7404), (15103-15108), |
| | (17365-17370) |
| T₆ | (1645-1650), (2175-2180), (2522-2527), (4113-4118), (9846-9851), (16880-16885) |
| G₆ | (5232-5237), (8307-8312), (17478-17483) |
| C₆ | (481-486), (8411-8416), (12662-12667) |
| A₇ | (15668-15674) |
| T₇ | (3849-3855), (5050-5056), (6762-6768), |
| A₈ | (913-920), (1050-1057), (3073-3080), (4161-4168), (4855-4862), (9447-9453), |
| | (13294-13300), (9587-9594), (16066-16073) |
| T₈ | (2320-2327), (5347-5354), (9832-9839) |
| T₉ | (5201-5209) |
| A₁₀ | (1000-1009), (1039-1048), |
| A₁₁ | (1081-1091) |
| (TC)₄ | (1108-1115) |
| (AC)₄ | (6835-6842) |
| (CT)₅ | (421-430) |
| (TC)₅ | (9995-10004) |
| (TCC)₄ | (4608-4619) |
| (CCT)₄ | (4828-4843) |
| (TTC)₅ | (240-254) |
| (CATG)₃ | (6560-6571) |
| (TTTC)₅ | (5638-5657) |
| (AAAGA)₃ | (12977-12991) |

The SSRs identified in the PDK_{—}30s1035941 scaffold (GenBank accession number: ACYX02056675-19615 nucleotides in length) and their respective locations in this scaffold are given in the following table:

| SSR | Positions occupées dans le scaffold PDK_30s1035941 |
|---|---|
| (A)₄ | (495-498), (647-650), (753-756), (1136-1139), (1309-1312), (1879-1882), (2582- |
| | 2585), (2605-2608), (2632-2635), (2705-2708), (2751-2754), (3050-3053), (2870- |
| | 2873), (3155-3158), (3190-3193), (3247-3250), (3603-3606), (3967-3970), (4051- |
| | 4054), (4098-4101), (4126- 4129), (4219-4222), (4345-4348), (4540-4543), (4556- |
| | 4559), (4693-4696), (5015-5018), (5043-5046), (6265-6268), (6590-6593), (6612- |
| | 6615), (6981-6984), (7070-7073), (7137-7140), (7218-7221), (7346-7349), (7471- |
| | 7474), (7726-7729), T4 (7823-7826), (8369-8372), (8392-8395), (8419-8422), |
| | (8496-8499), (8542-8545), (8576-8580), (8766-8769), (8913-8916), (9290-9293), |
| | (9322-9325), (9345-9348), (9495-9498), (9725-9728), (9797-9800), (9871-9874), |
| | (10749-10752), (10863-10866), (10908-10911), (10984-10987), (11039-11042), |
| | (11277-11280), (11531-11534), (11714-11717), (11736-11739), (11860-11863), |
| | (12071-12074), (12139-12142), (12279-12282), (12400-12403), (12532-12535), |
| | (12556-12559), (12762-12765), (12890-12893), (13461-13464), (13466-13469), |
| | (13644-13647), (13714-13717), (13763-13766), (13879-13882), (14425-14428), |
| | (14450-14453), (15309-15312), (15625-15628), (15635-15638), (15821-15824), |
| | (15854-15857),(15874-15877), (16243-16246), (16964-16967), (17760-17763), |
| | (17886-17889), (18055-18058), (18362-18365), (18608-18611), (18836-18839), |
| | (19107-19110), (19205-19208), (19291-19294), (19361-19364), (19589-19592) |
| T₄ | (167-170), (223-226), (312-315), (476-479), (581-584), (651-654), (759-762), (794- |
| | 797), (901-904), (1194-1197), (1854-1857), (2085-2088), (2091-2094), (2170- |
| | 2173), (2213-2216), (2220-2223), (2254-2257), (2259-2262), (2388-2391), (2513- |
| | 2516), (2839-2842), (2758-2761), (2946-2949), (3012-3015), (3177-3180), (3538- |
| | 3541), (3614-3617), (3686-3689), (3760-3763), (3988-3991), (4203-4206), (4295- |
| | 4298), (4319-4322), (4669-4672), (4702-4705), (4878-4881), (5034-5037), (5510- |
| | 5513), (6016-6019), (6086-6089), (6091-6094), (6320-6323), (6400-6403), (6449- |
| | 6452), (6517-6520), (6586-6589), (6622-6625), (6674-6677), (6920-6923), (7025- |
| | 7028), (6937-6940), (7163-7166), (7180-7183), (7273-7276), (7330-7333), (7498- |
| | 7501), (7535-7538), (7564-7567), (7601-7604), (7625-7628), (7956-7959), (8020- |
| | 8023), (8092-8095), (8117-8120), (8295-8298), (8549-8552), (8585-8588), (8630- |
| | 8633), (8669-8672), (8722-8725), (8748-8751), (8812-8815), (9031-9034), (9124- |
| | 9127), (9188-9191), (9316-9319), (9337-9340), (9402-9405), (9444-9447), (9516- |
| | 9519), (9559-9562), (9712-9715), (9773-9776), (9930-9933), (9944-9947), (10309- |
| | 10312), (10319-10322), (10393-10396), (10458-10461), (10463-10466), (10569- |
| | 10572), (10857-10860), (10877-10880), (11061-11064), (11195-11198), (11258- |
| | 11261), (11268-11271), (11464-11467), (11547-11550), (11680-11683), (11820- |
| | 11823), (11852-11855), (11947-11950), (12089-12093), (12178-12181), (12415- |
| | 12418), (12598-12601), (12617-12620), (12623-126260), (12767-127700), (12899- |
| | 12902), (12956-12959), (12980-12983), (13274-13277), (13290-13293), (13777- |
| | 13780), (13820-13823), (13905-13908), (13920-13923), (14046-14049), (14251- |
| | 14254), (14633-14636), (1467414677), (14690-14693), (14859-14862), (15406- |
| | 15409), (15584-15587), (15608-15611), (16261-16264), (16275-16278), (16307- |
| | 16310), (16572-16575), (16748-16751), (17383-17386), (18325-18328), (18948- |
| | 18952), (19051-19054) |
| G₄ | (1100-1103), (1334-1337), (2484-2487), (4453-4456), (6160-6163), (8266-8269), |
| | (10015-10018), (15510-15513), (16865-16868), (16922-16925), (17107-17110), |
| | (18292-18295), (1935419357), (19534-19537) |
| C₄ | (554-557), (2462-2465), (5659-5662), (5714-5717), (6173-6176), (6226-6229), |
| | (6596-6599), (6666-6669), (6736-6739), (6771-6774), (10667-10670), (14681- |
| | 14684), (15256-15259), (15288-15291), (15908-15911), (16057-16060), (16102- |
| | 16105), (16141-16144), (16167-16170), (16403-16406), (16581-16584), (18042- |
| | 18045), (18367-18370), (19460-19463), (19491-19494), (19509-19512), (19556- |
| | 19559) |
| A₅ | (853-857), (1763-1767), (1858-1862), (2115-2119), (2279-2283), (2785-2789), |
| | (3036-3040), (3256-3260), (3280-3284), (3389-3393), (4140-4144), (4661-4665), |
| | (4789-4793), (4913-4917), (5064-5068), (5095-5099), (5102-5106), (5241-5245), |
| | (5295-5299), (6928-6932), (7817-7821), (9025-9029), (9434-9438), (9466-9470), |
| | (10162-10166), (10439-104430), (12499-12503), (12813-12817), (12929-12933), |
| | (14406-14410), (14994-14998), (15591-15595), (15769-15773), (16691-16695), |
| | (16715-16719), (17780-17784), (19247-19251), |
| T₅ | (112-116), (534-538), (634-638), (747-751), (810-814), (837-841), (871-875), |
| | (1188-1192), (1206-1210), (1374-1378), (1781-1785), (2178-2182), (2244-2248), |
| | (4007-4011), (4046-4050), (5152-5156), (5426-5430), (5460-5464), (5610-5614), |
| | (5953-5957), (6144-6148), (6194-6198), (6382-6386), (6488-6492), (6502-6506), |
| | (6954-6958), (7278-7282), (7660-7664), (8132-8136), (8847-8851), (9326-9330), |
| | (9521-9525), (9549-9553), (9564-9568), (9587-9591), (10216-10220), (10451- |
| | 10455), (11117-11121), (12477-12481), (12751-12755), (13282-13286), (13496- |
| | 13500), (14030-14034), (15709-15713), (16529-16533), (16547-16551), (19252- |
| | 19256) |
| G₅ | (2208-2212), (12325-12329), (13945-13949), (15954-15958), (17154-17158), |
| | (17775-17779), (18274-18278) |
| C₅ | (3993-3997), (15243-15247), (16130-16134), (18257-18261) |
| A₆ | (741-746), (3021-3026), (3596-3601), (4480-4485), (7115-7120), (9274-9279), |
| | (12300-12305), (9694-9699), (10977-10982), (12437-12442), (18268-18273) |
| T₆ | (731-736), (6048-6053), (6479-6484), (9506-9511), (1189511900), (14000-14005) |
| C₆ | (17248-17253) |
| A₇ | (2684-2690), (2809-2815), (4509-4515), (8600-8606), (12548-12554) |
| T₇ | (719-725), (920-926), (2331-2337), (9922-9928), (11249-11255), (11800-11806), |
| | (12991-12997), (15614-15620) |
| A₈ | (11129-11136), (8474-8481) |
| T₈ | (3116-3123), (4622-4629), (7230-7237) |
| A₉ | (4681-4689) |
| T₉ | (7404-7412) |
| A₁₀ | (11213-11222), (15645-15654) |
| A₁₁ | (2654-2664) |
| T₁₁ | (7848-7858) |
| A₁₂ | (2519-2530), (8301-8312), (8442-8453) |
| (TA)₄ | (3129-3136), (4635-4642) |
| (AT)₅ | (5022-5031) |
| (TA)₇ | (5754 5767) |
| (AG)₄ | (5795-5802) |
| (TC)₆ | (9676-9687) |
| (CT)₆ | (14504-14511), (16216-16227) |
| (TTC)₉ | (1957-1983) |
| (AAC)₅ | (15856-15870) |
| (AATT)₃ | (7923-7934) |
| (AAAG)₃ | (11831-11842) |

The SSRs identified in the PDK_{—}30s950111 scaffold (GenBank accession number: ACYX02030057-42207 nucleotides in length) and their respective locations in this scaffold are given in the following table:

| SSR | Positions occupées dans le scaffold PDK_30s950111 |
|---|---|
| A₄ | (557-560), (1541-1544), (1794-1797), (1833-1836), (1868-1871), (2334-2337), |
| | (2340-2343), (2479-2482), (2704-2707), (3044-3047),(3332-3335), (3396-3399), |
| | (3425-3428), (3473-3476), (3516-3519), (3558-3561),(3667-3670), (3690-3693), |
| | (4119-4122), (4246-4249), (4431-4434), (5247-5250),(5592-5595), (6049-6052), |
| | (6345-6348), (6778-6781), (6889-6892), (7067-7070),(7081-7084), (7181-7184), |
| | (7306- 7309),(7325- 7328),(7600-7603), (7662-7665),(8069-8072), (8886-8889), |
| | (9159-9162), (9331-9334), (9498-9501), (9537-9540),(9658-9661), (9757-9760), |
| | (9945-9948), (10166-10169), (10423-10426), (10695-10698), (10879-10882), |
| | (10904-10907), (11665-11668), (11676-11679), (11789-11792), (11910-11913), |
| | (12124-12127), (12820-12823), (13066-13069), (13083-13086), (13556-13559), |
| | (13707-13710), (13869-13872), (13886-13889), (14310-14313), (14575-14578), |
| | (14920-14923), (15081-15084), (15426-15429), (15611-15614), (15677-15680), |
| | (15872-15875), (16163-16166), (16192-16195), (16373-16376), (16554-16557), |
| | (16725-16728), (17191-17194), (17253-17256), (17405-17408), (17596-17599), |
| | (17629-17632), (17884-17887), (17985-17988), (18103-18106), (18143-18146), |
| | (18179-18182), (18401-18404), (18820-18823), (18899-18902), (18948-18951), |
| | (19399-19402), (19531-19534), (19564-19567), (19674-19677), (19708-19711), |
| | (19713-19716), (19794-19797), (20067-20070), (20128-20131), (20169-20172), |
| | (20202-20205), (20400-20403), (20671-20674), (21198-21201), (21336-21339), |
| | (21592-21595), (22339-22342), (22413-22416), (23142-23145), (23186-23189), |
| | (23279-23282), (23343-23346), (23539-23542), (23650-23653), (23753-23756), |
| | (23763-23766), (23934-23937), (24027-24030), (24052-24055), (24349-24352), |
| | (24591-24594), (24875-24878), (24960-24963), (25001-25004), (26089-26092), |
| | (26174-26177), (26650-26653), (27217-27220), (27624-27627), (27630-27633), |
| | (27732-27735), (28084-28087), (28175-28178), (28241-28244), (28266-28269), |
| | (28297-28300), (28319-28322), (28369-28372), (28593-28596), (28705-28708), |
| | (29936-29939), (30017-30020), (30280-30283), (30321-30324), (30570-30573), |
| | (30777-30780), (30903-30906), (30936-30939), (30967-30970), (30980-30983), |
| | (31028-31031), (31264-31267), (31497-31500), (32044-32047), (32236-32239), |
| | (32246-32249), (32251-32254), (32365-32368), (32405-32408), (32639-32642), |
| | (32787-32790), (32895-32898), (33041-33044), (33264-33267), (33500-33503), |
| | (33632-33635), (33991-33994), (34219-34222), (34234-34237), (34420-34423), |
| | (34439-34442), (34520-34523), (34535-34538), (34670-34673), (34900-34903), |
| | (34926-34929), (34980-34983), (35030-35033), (35226-35229), (35355-35358), |
| | (35548-35551), (35616-35619), (35675-35678), (35681-35684), (35690-35693), |
| | (36013-36016), (36093-36096), (36360-36363), (36568-36571), (37070-37073), |
| | (37078-37081), (37115-37118), (37164-37167), (37197-37200), (37362-37365), |
| | (37409-37412), (37414-37417), (37486-37489), (37521-37524), (37717-37720), |
| | (37910-37913), (37943-37946), (38348-38351), (38540-38543), (38597-38600), |
| | (38693-38696), (39190-39193), (39453-39456), (40216-40219), (40917-40920), |
| | (41537-41540), (41633-41636), (41949-41952), (41956-41959), (41987-41990) |
| T₄ | (109-112), (218-221), (269-272), (405-408), (411-414), (429-432), (459-462), (481- |
| | 484), (826-829), (844-847), (849-852), (989-992), (1161-1164), (1220-1223), |
| | (1227-1230), (1263-1266), (1396-1399), (1419-1422), (1443-1446), (1558-1561), |
| | (1651-1654), (1755-1758), (1801-1804), (2185-2188), (2567-2570), (2683-2686), |
| | (2948-2951), (3028-3031), (3292-3295), (3383-3386), (3451-3454), (3481-3484), |
| | (3524-3527), (3720-3723), (3851-3854), (3911-3914), (4160-4163), (4226-4229), |
| | (4254-4257), (4440-4443), (4507-4510), (4580-4583), (4676-4679), (5096-5099), |
| | (5268-5271), (5369-5372), (5499-5502), (5665-5668), (5738-5741), (5765-5768), |
| | (5847-5850), (6009-6012), (6259-6262), (7075 -7078), (7099-7102), (7431-7434), |
| | (7456-7459), (7720-7723), (7778-7781), (8543-8546), (8646-8649), (8969-8972), |
| | (9005-9008), (9039-9042), (9204-9207), (9788-9791), (9868-9871), (10280- |
| | 10283), (10853-10856), (10931-10934), (10939-10942), (11058-11061), (11297- |
| | 11300), (11422-11425), (11591-11594), (11695-11698), (11724-11727), (11826- |
| | 11829), (11833-11836), (11870-11873), (11946-11949), (12086-12089), (12300- |
| | 12303), (12414-12417), (12610-12613), (12726-12729), (12960-12963), (13160- |
| | 13163), (13211-13214), (13711-13714), (13751-13754), (13850-13853), (13928- |
| | 13931), (14149-14152), (14304-14307), (14636-14639), (14643-14646), (14674- |
| | 14677), (14680-14683), (14715-14718), (15006-15009), (15135-15138), (15219- |
| | 15222), (15293-15296), (15309-15312), (15375-15378), (15393-15396), (15412- |
| | 15415), (15699-15702), (15882-15885), (15961-15964), (16044-16047), T4 |
| | (16263-16266), (16422-16425), (16584-16587), (16610-16613), (16627-16630), |
| | (16693-16696), (16799-16802), (16922-16925), (16931-16934), (16984-16987), |
| | (17118-17121), (17323-17326), (17329-17332), (17415-17418), (17444-17447), |
| | (17467-17470), (17573-17576), (17907-17910), (18261-18264), (18354-18357), |
| | (18540-18543), (18556-18559), (18655-18658), (18773-18776), (18778-18781), |
| | (19016-19019), (19063-19066), (19175-19178), (19367-19370), (19394-19397), |
| | (19449-19452), (19495-19498), (19500-19503), (19653-19656), (1966619669), |
| | (19785-19788), (20222-20225), (20244-20247), (20328-20331), (20598-20601), |
| | (20662-20665), (20810-20813), (20881-20884), (21014-21017), (21258-21261), |
| | (21319-21322), (21578-21581), (21746-21749), (21778-21781), (21931-21934), |
| | (22066-22069), (22078-22081), (22104-22107), (22112-22115), (22251-22257), |
| | (22404-22407), (22482-22485), (22543-22546), (23035-23038), (23148-23151), |
| | (23323-23326), (23333-23336), (23446-23449), (23496-23499), (23644-23647), |
| | (24147-24150), (24323-24326), (24637-24640), (24656-24659), (24668-24671), |
| | (25115-25118), (25224-25227), (25266-25269), (25579-25582), (25968-25971), |
| | (27111-27114), (27150-27153), (27207-27210), (27918-27921), (27947-27950), |
| | (27963-27966), (28069-28072), (28150-28153), (28205-28208), (28215-28218), |
| | (28510-28513), (28810-28813), (28837-28840), (29876-29879), (29892-29895), |
| | (30032-30035), (30130-30133), (30142-30145), (30302-30305), (30366-30369), |
| | (30492-30495), (30971-30974), (31052-31055), (31120-31123), (31125-31128), |
| | (31191-31194), (31387-31390), (31454-31457), (31561-31564), (31709-31712), |
| | (31774-31777), (31820-31823), (31893-31896), (32086-32089), (32097-32100), |
| | (32397-32400), (32561-32564), (32599-32602), (32672-32675), (32687-32690), |
| | (33145-33148), (33284-33287), (33735-33738), (34106-34109), (34254-34257), |
| | (34357-34360), (34514-34517), (34718-34721), (34811-34814), (34865-34868), |
| | (35024-35027), (35401-35404), (36221-36224), (36247-36250), (36396-36399), |
| | (36785-36788), (37092-37095), (37437-37440), (37482-37485), (37570-37573), |
| | (37630-37633), (37662-37665), (37830-37833), (38032-38035), (38064-38067), |
| | (38380-38383), (38736-38739), (39000-39003), (39005-39008), (39619-39622), |
| | (39664-39667), (39703-39706), (39741-39744), (39753-39756), (40401-40404), |
| | (40514-40517), (40775-40778), (41201-41204), (41450-41453), (41613-41616), |
| | (41817-41820), (42032-42035), (42055-42058) |
| G₄ | (2745-2748), (2766-2769), (2879-2882), (2948-2951), (3166-3169), (3574-3577), |
| | (4357-4360), (4903-4906), (5046-5049), (5338-5341), (6230-6233), (8891-8894), |
| | (9236-9239), (9304-9307), (11120-11123), (13215-13218), G4 (16825-16828), |
| | (18323-18326), (18982-18985), (20414-20417), (21018-21021), (21529-21532), |
| | (22455-22458), (27923-27926), (28015-28018), (29474-29477), (29567-29570), |
| | (30940-30943), (32409-32412), (32526-32529), (33662-33665), (35389-35392), |
| | (35727-35730), (36191-36194), (37251-37254), (37496-37499), (38544-38547), |
| | (39630-39633), (40683-40686) |
| C₄ | (4312-4315), (4796-4799), (5028-5031), (5615-5618), (8448-8451), (8590-8593), |
| | (10478-10481), (10492-10495), (10582-10585), (10833-10836), (13219-13222), |
| | (15146-15149), (16239-16242), (16460-16463), (18127-18130), (20611-20614), |
| | (23026-23029), (29217-29220), (30096-30099), (30263-30266), (31154-31157), |
| | (31608-31611), (33122-33125), (34461-34464), (35847-35850), (40323-40326), |
| | (40800-40803), (41504-41507) |
| A₅ | (35-39), (535-539), (854-858), (1585-1589), (2203-2207), (2696-2700), (3937- |
| | 3941), (4236-4240), (4297-4301), (4697-4701), (5445-5449), (6441-6445), (7369- |
| | 7373), (8090-8094), (8568-8572), (9971-9975), (10177-10181), (10951-10955), |
| | (11973-11977), (12373-12377), (12519-12523), (13563-13567), (14696-14700), |
| | (14969-14973), (15399-15403), (15562-15566), (15843-15847), (17185-17189), |
| | (17541-17545), (17667-17671), (17710-17714), (17967-17971), (19190-19194), |
| | (20085-20089), (21597-21601), (22777-22781), (23201-23205), (23287-23291), |
| | (23406-23410), (24077-24081), (24424-24428), (24796-24800), (25394-25398), |
| | (25626-25630), (26726-26730), (28089-28093), (28698-28702), (30001-30005), |
| | (30066-30070), (30267-30271), (31569-31573), (31883-31887), (32262-32266), |
| | (32644-32648), (32846-32850), (33466-33470), (33883-33887), (34425-34429), |
| | (34723-34727), (34793-34797), (34803-34807), (34909-34913), (35643-35647), |
| | (35695-35699), (35882-35886), (35891-35895), (36499-36503), (39467-39471), |
| | (39559-39563), (39851-39855), (40085-40089), (40602-40606), (40927-40931), |
| | (41166-41170), (41650-41654), (41981-41985) |
| T₅ | (188-191), (341-345), (435-439), (453-457), (510-514), (706-710), (756-760), (792- |
| | 796), (1119-1123), (2049-2053), (2087-2091), (2117-2121), (2156-2160), (2519- |
| | 2523), (2526-2530), (2538-2542), (2606-2610), (3156-3160), (3204-3208), (3268- |
| | 3272), (3317-3321), (3435-3439), (3545-3549), (5386-5390), (6908-6912), (7132- |
| | 7136), (7295-7299), (7728-7732), (7735-7739), (7745-7749), (7767-7771), (9465- |
| | 9469), (9683-9687), (10963-10967), (11063-11067), (11232-11236), (11253- |
| | 11257), (11336-11340), (11626-11630), (11729-11733), (12505-12509), (12735- |
| | 12739), (12832-12836), (13475-13479), (14660-14664), (15152-15156), (15233- |
| | 15237), (15467-15471), (15895-15899), (16201-16205), (16698-16702), (17074- |
| | 17078), (17291-17295), (17564-17568), (17635-17639), (17957-17961), (18216- |
| | 18220), (18570-18574), (18807-18811), (18923-18927), (19471-19475), (19599- |
| | 19603), (19772-19776), (20615-20619), (20636-20640), (20909-20913), (21772- |
| | 21776), (24098-24102), (24839-24843), (25137-25141), (25975-25979), (26797- |
| | 26801), (27991-27995), (28136-28140), (28312-28316), (29101-29105), (29350- |
| | 29354), (29857-29861), (30009-30013), (30337-30341), (30360-30364), (31281- |
| | 31285), (32483-32487), (32659-32663), (32781-32785), (33295-33299), (33520- |
| | 33524), (34020-34024), (37150-37154), (37182-37186), (37458-37462), (37491- |
| | 37495), (37754-37758), (38269-38273), (38676-38680), (38894-38898), (40875- |
| | 40879) |
| G₅ | (3094-3098), (4740-4744), (5380-5384), (5485-5488), (8301-8305), (11000- |
| | 11003), (12570-12573), (13902-13906), (14529-14533), (21444-21448), (23411- |
| | 23414), (26626-26630), (32071-32075), (40006-40010), (40046-40050), (40096- |
| | 40100), (40694-40698) |
| C₅ | (1943-1947), (3715-3719), (10107-10111), (10504-10508), (10513-10517), (26000- |
| | 26004), (40264-40268) |
| A₆ | (1380-1385), (1759-1764), (2348-2353), (3536-3541), (4335-4340), (6470-6475), |
| | (7330-7335), (7692-7697), (9395-9400), (9613-9618), (12944-12949), (13465- |
| | 13470), (13767-13772), (17125-17130), (17494-17499), (17897-17902), (19538- |
| | 19543), (23822-23827), (28306-28311), (32954-32959), (33202-33207), (34312- |
| | 34317), (34687-34692), A6 (39548-39553) |
| T₆ | (252-257), (717-722), (837-842), (2209-2214), (2636-2641), (2659-2664), (5166- |
| | 5171), (6236-6241), (7110-7115), (7418-7423), (9735-9740), (10486-10491), |
| | (13757-13762), (14442-14447), (15792-15797), (16348-16353), (16500-16505), |
| | (17102-17107), (19081-19086), (19442-19447), (19639-19644), (20263-20268), |
| | (20579-20584), (21751-21756), (21758-21763), (22594-22599), (23943-23948), |
| | (28076-28081), (28142-28147), (28692-28697), (33251-33256), (33935-33940), |
| | (34380-34385), (34950-34955), (35968-35973), (39417-39422), (40829-40834), |
| | (41225-41230), (41472-41477) |
| G₆ | (4949-4954) |
| C₆ | (25360-25365) |
| A₇ | (3122-3128), (12583-12589), (13257-13263), (17387-17393), (22293-22296), |
| | (23721-23727), (27642-27648), (30195-30201), (32228-32234), (33854-33860), |
| | (34301-34307), (34855-34861), (42154-42160) |
| T₇ | (4096-4102), (7345-7351), (13655-13661), (15114-15120), (15647-15653), (17200- |
| | 17206), (19161-19167), (25663-25669), (28258-28264), (29733-29739), (34322- |
| | 34328), (40524-40530) |
| A₈ | (21392-21399), (27904-27911), (32581-32588), (34264-34271), (36522-36529) |
| T₈ | (7705-7712), (9644-9651), (10863-10870), (14685-14692), (20723-20730), (25474- |
| | 25481), (32604-32611), (39457-39464), (40844-40851), (40906-40913) |
| A₉ | (16005-16013) |
| T₉ | (3239-3247), (11046-11054), (12428-12436), (19026-19034), (32122-32130) |
| A₁₀ | (16094-16103), (36678-36687) |
| T₁₀ | (27882-27891), (41251-41260) |
| A₁₁ | (10237-10247), (33842-33852) |
| A₁₂ | (36665-36676) |
| T₁₆ | (42177-42192) |
| T₁₇ | (15901-15917) |
| A₁₈ | (7475-7492) |
| (CA)₄ | (1906-1913) |
| (AG)₄ | (7826-7833) |
| (CT)₄ | (7894-7901), (10785-10792), (18533-18540), (29846-29853), (40863-40870) |
| (AT)₄ | (17484-17491), (19623-19630), (30869-30876) |
| (TC)₄ | (18333-18340), (31359-31366) |
| (TA)₄ | (20027-20034) |
| (GA)₄ | (40012-40019) |
| (AG)₅ | (1890-1899) |
| (GA)₅ | (40155-40164) |
| (AG)₇ | (40577-40590) |
| (AG)₅ | (37256-37271) |
| (AT)₉ | (9014-9031) |
| (TGT)₄ | (630-641) |
| (GAG)₁₂ | (39771-39806) |
| (ATCC)₃ | (10560-10571) |
| (ACTA)₃ | (25750-25761) |

The SSRs identified in the PDK_{—}30s684341 scaffold (GenBank accession number: ACYX02093312-3946 nucleotides in length) and their respective locations in this scaffold are given in the following table:

| SSR | Positions occupées dans le scaffold PDK_30s684341 |
|---|---|
| A₄ | (135-138), (922-925), (996-999), (1134-1137), (1540-1543), (1660-1663), (1735- |
| | 1738), (2060-2063), (2152-2155), (2423-2426), (2744-2747), (2754-2757), (2816- |
| | 2819), (2845-2848), (2948-2951), (3112-3115), (3218-3221), (3248-3251), (3276- |
| | 3279), (3369-3372), (3452-3455), (3733-3736), (3739-3742), (3767-3770) |
| T₄ | (197-200), (376-379), (405-408), (418-421), (521-524), (1647-1650), (1763-1766), |
| | (2004-2007), (2075-2078), (2242-2245), (2497-2500), (2836-2839), (2873-2876), |
| | (3047-3050), (3272-3275), (3407-3410), (3465-3468) |
| G₄ | (3082-3085), (3328-3331) |
| C₄ | (21-24), (162-165), (400-403), (1656-1659), (3066-3069) |
| A₅ | (291-295), (553-557), (653-657), (1067-1071), (1714-1718), (2978-2982), (3705- |
| | 3709), (3725-3729), (3748-3752) |
| T₅ | 1430-1434), (1933-1937), (2316-2320), (3004-3008) |
| G₅ | (3912-3916) |
| C₅ | (3526-3530) |
| A₆ | (1745-1750), (3149-3154), (3180-3185), (3491-3496), (3919-3924) |
| T₆ | (394-399), (3420-3425) |
| A₇ | (530-536), (2233-2239) |
| T₇ | (2721-2727) |
| A₉ | (1005-1013), (2728-2735) |
| (CT)₄ | (2705-2712) |

The SSRs identified in the PDK_{—}30s754231 scaffold (GenBank accession number: ACYX02065374-14607 nucleotides in length) and their respective locations in this scaffold are given in the following table:

| SSR | Positions occupées dans le scaffold PDK_30s754231 |
|---|---|
| A₄ | (81-84),(2770-2773), (2882-2885), (2991-2994), (3115-3118), (3122-3125),(3326- |
| | 3329), (3459-3462), (3879-3882), (3972-3975), (4743-4746), (4820-4823), (4827- |
| | 4830), (4973-4976), (4978-4981), (5157-5160), (5332-5335), (5538-5541), (5712- |
| | 5715), (5958-5961), (5969-5972), (5982-5985), (5987-5990), (6001-6004), (6006- |
| | 6009), (6029-6032), (6050-6053), (6055-6058), (6284-6287), (6428-6431), (6646- |
| | 6649), (7049-7052), (7202-7205), (7485-7488), (7809-7812), (8338-8341), (8537- |
| | 8540), (8545-8548), (8679-8682), (8708-8711), (8726-8729), (8745-8748), (8776- |
| | 8779), (9517-9520), (9952-9955), (10067-10070), (10132-10135), (10524-10527), |
| | (10826-10829), (10933-10936), (10975-10978), (11077-11080), (11550-11553), |
| | (11738-11741), (11820-11823), (11836-11839), (2001-12004), (12291-12294), |
| | (12341-12344), (12418-12421), (12724-12727), (12859-12862), (13084-13087), |
| | (13313-13316), (13347-13350), (13436-13439), (13510-13513), (13566-13569), |
| | (13599-13602), (13629-13632), (13746-13749), (13896-13899), (13971-13974), |
| | (14015-14018), (14374-14377), (14428-14431) |
| T₄ | (102-105), (509-512), (577-580), (1221-1224), (2252-2255), (2297-2300), (2633- |
| | 2636), (2684-2687), (2692-2695), (2725-2728), (809-2812), (2929-2932), (3257- |
| | 3260), (3276-3279), (3677-3680), (3709-3712), (3727-3730), (3732-3735), (3796- |
| | 3799), (3967-3970), (4091-4094), (4385-4388), (4696-4699), (4723-4726), (4947- |
| | 4950), (4998-5001), (5014-5017), (5043-5046), (5273-5276), (5448-5451), (5475- |
| | 5478), (6041-6044), (6212-6215), T4 (6303-6306), (6487-6490), (6544-6547), |
| | (6556-6559), (6806-6809), T4 (7053-7056), (7394-7397), (7978-7981), (8352- |
| | 8355), (8640-8643), (8730-8733), (9033-9036), (9047-9050), (9699-9702), (9787- |
| | 9790), (9959-9962), (9978-9981), (10187-10190), (10315-10318), (10350-10353), |
| | (10373-10376), (10679-10682), (10688-10691), (10718-10721), (10834-10837), |
| | (10950-10953), (10968-10971), (10979-10982), (10995-10998), (11009-11012), |
| | (11185-11188), (11244-11247), (11282-11285), (11291-11294), (11414-11417), |
| | (11446-11449), (11593-11596), (11619-11622), (11898-11911), (11946-11949), |
| | (11961-11964), (12051-12054), (12137-12140), (12233-12236), (12532-12535), |
| | (12686-12689), (12742-12745), (12811-12814), (12818-12821), (13058-13061), |
| | (13168-13171), (13185-13188), (13799-13802), (13916-13919), (13943-13946), |
| | (13967-13970), T4 (14165-14168), (14225-14228), (14313-14316) |
| G₄ | (89-92), (966-969), (1003-1006), (1017-1020), (1034-1037), (1228-1231), (1307- |
| | 1310), (1416-1419), (1585-1588), (1653-1656), (1909-1912), (2170-2173), (2479- |
| | 2482), (2646-2649), (3053-3056), (4348-4351), (5722-5725), (5809-5812), (6499- |
| | 6502), (7196-7199), (7316-7319), (7388-7391), (7459-7462), (7566-7569), (7597- |
| | 7600), (7613-7616), (7635-7638), (8106-8109), (8135-8138), (8344-8347), (8395- |
| | 8398), (8430-8433), (8480-8483), (10810-10813), (2869-12872), (13257-13260) |
| C₄ | (106-109), (583-586), (613-616), (660-663), (1429-1432), (1597-1600), (2174- |
| | 2177), (13146-13149), (14119-14122), (14291-14294), (14333-14336) |
| A₅ | (2732-2736), (3437-3441), (5383-5387), (6023-6027), (6223-6227), (6327-6331), |
| | (6907-6911), (6921-6925), (6958-6962), (7003-7007), (7476-7480), (10109- |
| | 10113), (10477-10481), (11105-11109), (12019-12023), (13852-13856), (13901- |
| | 13905) |
| T₅ | 624-628), (1888-1892), (1959-1963), (2056-2060), (2668-2672), (2678-2682), |
| | (2785-2789), (3309-3313), (3639-3643), (3866-3870), (4489-4493), (4769-4773), |
| | (5126-5130), (5558-5562), (5729-5733), (7042-7046) , (7642-7646), (7648-7652), |
| | (7696-7700), (7719-7723), (8245-8249), (8994-8998), (9231-9235), (9326-9330), |
| | (9538-9542), (10470-10474), (10756-10760), (10774-10778), (10792-10796), |
| | (11716-11720), (13283-13287), (13603-13607), (13995-13999), (14079-14083) |
| G₅ | (461-465), (1248-1252), (1496-1500), (1742-1746), (1818-1822), (2485-2489), |
| | (7982-7986), (8141-8145), (9193-9197), (12527-12531), (12549-12553) |
| C₅ | (2076-2080), (2223-2227) |
| A₆ | (7768-7773), (8750-8755), (12480-12485), (13019-13024), (13101-13106), (13235- |
| | 13240), (13843-13848) |
| T₆ | (2896-2901), (6529-6534), (7712-7717), (8761-8766), (9111-9116), (10200- |
| | 10205), (11433-11438), (11599-11604), (11610-11615), (11871-11876), (12304- |
| | 12309), (12466-12471), (12959-12964) |
| A₇ | (8700-8706), (10034-10040) |
| T₇ | (112-118), (4902-4908), (8359-8365), (9279-9285), (12279-12285), (13502-13508) |
| T₈ | (10058-10065) |
| G₈ | (1945-1952) |
| T₁₀ | (8368-8377) |
| T₁₁ | (9096-9106) |
| T₁₂ | (6811-6822) |
| (GT)₄ | (2541-2548), (7279-7286) |
| (AT)₄ | (6881-6888), |
| (GA)₅ | (7515-7524) |
| (GT)₅ | (7906-7915) |
| (CT)₆ | (7878-7889) |
| (GA)₆ | (9715-9726) |
| (CT)₇ | (5169-5182) |
| (AG)₇ | (7488-7501) |
| (GAC)₃ | (13039-13053) |
| (TAG)₄ | (3987-3998), |
| (AGA)₄ | (8558-8569) |
| (TGG)₅ | (8405-8419) |
| (AGA)₁₃ | (5821-5863) |
| (GGAG)₃ | (7506-7517) |
| (AAATA)₃ | (6046-6060) |

The SSRs identified in the PDK_{—}30s65509204 scaffold (GenBank accession number: ACYX02069820-12637 nucleotides in length) and their respective locations in this scaffold are given in the following table:

| SSR | Positions occupées dans le scaffold PDK_30s65509204 |
|---|---|
| A₄ | (56-59), (2882-2885), (2887-2890), (2944-2947), (2989-2992), (3038-3041), |
| | (3620-3623), (3983-3986), (3992-3995), (4097-4100), (4124-4127), (4208- |
| | 4211), (4251-4254), (4364-4367), (4398-4401), (4490-4493), (4590-4593), |
| | (4610-4613), (4795-4798), (4979-4982), (5201-5204), (5303-5306), (5340- |
| | 5343), (5463-5466), (5709-5712), (5785-5788), (6116-6119), (6150-6153), |
| | (6309-6312), (6383-6386), (6556-6559), (6892-6895), (7276-7279), (7306- |
| | 7309), (7321-7324), (7652-7655), (8067-8070), (8263-8266), (8518-8521), |
| | (8550-8553), (8677-8680), (8682-8685), (8883-8886), (8948-8951), (9000- |
| | 9003), (9271-9274), (9299-9302), (9395-9398), (9421-9424), (9484-9487), |
| | (9635-9638), (9928-9931), (10084-10087), (10097-10100), (10351-10354), |
| | (10473-10476), (10533-10536), (10589-10592), (10712-10715), (11529-11532), |
| | (11937-11940), (12045-12048), (12309-12312), (12519-12522) |
| T₄ | (196-199), (516-519), (570-573), (1654-1657), (1971-1974), (2243-2246), |
| | (2734-2737), (2781-2784), (2838-2841), (2958-2961), (3128-3131), (3160- |
| | 3163), (3314-3317), (3380-3383), (3385-3388), (3611-3614), (3801-3804), |
| | (3830-3833), (3914-3917), (3919-3922), (3996-3999), (4059-4062), (4257- |
| | 4260), (4438-4441), (4533-4536), (4632-4635), (4753-4756), (4776-4779), |
| | (5083-5086), (5125-5128), (5441-5444), (5504-5507), (5582-5585), (5619- |
| | 5622), (5624-5627), (5796-5799), (5803-5806), (5881-5884), (5892-5895), |
| | (6035-6038), (6073-6076), (6130-6133), (6158-6161), (6256-6259), (6291- |
| | 6294), (6408-6411), (6437-6440), (6457-6460), (6527-6530), (6658-6661), |
| | (6845-6848), (6964-6967), (7284-7287), (7840-7843), (7857-7860), (7868- |
| | 7871), (7917-7920), (8059-8062), (8122-8125), (8162-8165), (8279-8282), |
| | (8524-8527), (8641-8644), (8960-8963), (8993-8996), (9263-9266), (9405- |
| | 9408), (9515-9518), (9660-9663), (9674-9677), (9782-9785), (9792-9795), |
| | (9897-9900), (9943-9946), (10009-10012), (10855-10858) |
| G₄ | (506-509), (808-811), (936-939), (1278-1281), (1869-1872), (2271-2274), |
| | (2390-2393), (2408-2411), (2471-2474), (2597-2600), (3397-3400), (3406- |
| | 3409), (4212-4215), (5023-5026), (7116-7119), (7611-7614), (9249-9252), |
| | (10546-10549), (11252-11255), (11324-11327) |
| C₄ | (737-740), (1696-1699), (1835-1838), (2609-2612), (5377-5380), (7624-7627), |
| | (8270-8273), (9324-9327), (10125-10128), (10182-10185), (10221-10224), |
| | (10432-10435), (10486-10489), (10933-10936), (11590-11593), (11647-11650), |
| | (11670-11673), (11682-11685), (11933-11936), (11962-11965), (12020-12023), |
| | (12166-12169), (12259-12262), (12331-12334) |
| A₅ | (2867-2871), (3067-3071), (3761-3765), (5313-5317), (5686-5690), (5739- |
| | 5743), (6017-6021), (6780-6784), (6860-6864), (8651-8655), (9415-9419), |
| | (9435-9439), (9683-9687), (9706-9710), (10091-10095), (10560-10564), |
| | (10594-10598), (12275-12279) |
| T₅ | (147-151), (90-94), (147-151), (758-762), (2106-2110), (2964-2968), (3046- |
| | 3050), (3208-3212), (3366-3370), (3567-3571), (3789-3793), (3977-3981), |
| | (4091-4095), (4347-4351), (4480-4484), (4860-4864), (5467-5471), (5815- |
| | 5819), (6208-6212), (6971-6975), (6998-7002), (7020-7024), (7466-7470), |
| | (7830-7834), (8112-8116), (8174-8178), (8187-8191) |
| G₅ | (51-559), (338-342), (732-736), (971-975), (1198-1202), (4199-4203), (9048- |
| | 9052), (9873-9877) |
| C₅ | (630-634), (2324-2328), (3137-3141), (10230-10234), (10290-10294), |
| | (10322-10326) |
| A₆ | (2858-2863), (4403-4408), (6277-6282), (6791-6796), (9862-9867), (11210- |
| | 11215), (11436-11441) |
| T₆ | (344-349), (485-490), (2773-2778), (3025-3030), (3079-3084), (6495-6500), |
| | (6506-6511), (7055-7060) |
| G₆ | (1618-1623), (2580-2585) |
| C₆ | (3190-3195), (11204-11209) |
| T₇ | (2970-2976), (4152-4158), |
| G₇ | (1703-1709) |
| A₈ | (2873-2880), A8 (5954-5961) |
| A₉ | (4893-4901) |
| T₉ | (3475-3483), (4837-4845), (5148-5156), (5945-5953), (6417-6425), (6804- |
| | 6812), (10040-10048) |
| T₁₁ | (3946-3956) |
| A₁₂ | (2845-2856) |
| (CT)₄ | (3195-3202) |
| (TA)₄ | (3749-3756) |
| (TC)₃ | (3263-3288), |
| (TTA)₄ | (8100-8111) |
| (TCT)₅ | (10022-10036) |
| (ATTT)₃ | (5510-5521) |
| (CCAT)₃ | (11476-11487) |
| (AAAAG )₃ | (2877-2891) |

The SSRs identified in the PDK_{—}30s667171 scaffold (GenBank accession number: ACYX02067687-13542 nucleotides in length) and their respective locations in this scaffold are given in the following table:

| SSR | Positions occupées dans le scaffold PDK_30s667171 |
|---|---|
| A₄ | (165-168), (338-341), (952-955), (1151-1154), (1799-1802), (1980-1983), (2032- |
| | 2035), (2076-2079), (2096-2099), (2509-2512), (3298-3301), (3311-3314), (3397- |
| | 3400), (3408-3411), (4153-4156), (4215-4218), (4247-4250), (4744-4747), (5226- |
| | 5229), (5750-5753), (6149-6152), (6260-6263), (6998-7001), (7212-7215), (7475- |
| | 7478), (7488-7491), (7891-7894), (7939-7942), (8065-8068), (8438-8441), (8465- |
| | 8468), (8909-8912), (9295-9298), (9323-9326), (9503-9506), (10111-10114), |
| | (10183-10186), (10189-10192), (10249-10252), (10356-10359), (10747-10750), |
| | (10832-10835), (10854-10857), (10859-10862), (10887-10890), (10892-10895), |
| | (11300-11303), (11429-11432), (11462-11465), (11807-11810), (11982-11985), |
| | (12059-12062), (12116-12119), (12127-12130), (12196-12199), (12253-12256), |
| | (12327-12330), (12340-12343), (12351-12354), (12519-12522), (12589-12592), |
| | (13130-13133), (13437-13440), (13492-13495) |
| T₄ | (297-300), (534-537), (712-715), (936-939), (991-994), (1302-1305), (1631-1634), |
| | (1665-1668), (1874-1877), (1923-1926), (1934-1937), (2036-2039), (2080-2083), |
| | (2211-2214), (2252-2255), (2341-2344), (2860-2863), (2910-2913), (2942-2945), |
| | (3036-3039), (3058-3061), (3441-3444), (3607-3610), (3660-3663), (3728-3731), |
| | (3747-3750), (4004-4007), (4018-4021), (4041-4044), (4051-4054), (4127-4130), |
| | (5008-5011), (5059-5062), (5162-5165), (5208-5211), (5391-5394), (5444-5447), |
| | (5573-5576), (5581-5584), (5635-5638), (5685-5688), (5736-5739), (5850-5853), |
| | (6094-6097), (6361-6364), (6394-6397), (6649-6652), (7297-7300), (7539-7542), |
| | (8514-8517), (8601-8604), (9153-9156), (9255-9258), (9373-9376), (9480-9483), |
| | (9640-9643), (9690-9693), (9756-9759), (9867-9870), (10024-10027), (10155- |
| | 10158), (10576-10579), (10945-10948), (11372-11375), (11388-11391), (11573- |
| | 11576), (11730-11733), (11789-11792), (11814-11817), (11917-11920), (12012- |
| | 12015), (12273-12276), (12483-12486), (12789-12792), (12813-12816), (12836- |
| | 12839), (13108-13111), (13113-13116), (13196-13199), (13256-13259), |
| | (13323-13326) |
| G₄ | 315-318), (1020-1023), (1074-1077), (2292-2295), (2546-2549), (4603-4606), |
| | (4737-4740), (4843-4846), (4905-4908), (6270-6273), (6759-6762), (6935-6938), |
| | (7745-7748), (7816-7819), (8898-8901), (9203-9206), (9430-9433), (9752-9755), |
| | (9863-9866), (9939-9942), (10236-10239), (10510-10513), (11052-11055), (11534- |
| | 11537), (12384-12387), (12443-12446), (12494-12497), (13336-13339) |
| C₄ | (104-107), (330-333), (349-352), (663-666), (686-689), (1085-1088), (1179-1182), |
| | (2655-2658), (2824-2827), (3003-3006), (5403-5406), (6549-6552), (7346-7349), |
| | (10465-10468), (12095-12098), (12395-12398) |
| A₅ | (283-287), (2126-2130), (3272-3276), (3508-3512), (3988-3992), (4094-4098), |
| | (4326-4330), (5116-5120), (5935-5939), (6300-6304), (8365-8369), (9896-9900), |
| | (10376-10380), (10541-10545), (10700-10704), (11105-11109), (11418-11422), |
| | (12173-12177), (12530-12534), (12561-12565), (12793-12797), (13180-13184) |
| T₅ | (392-396), (969-973), (1183-1187), (1645-1649), (1928-1932), (2880-2884), (3258- |
| | 3262), (3485-3489), (4239-4243), (4624-4628), (5043-5047), (5374-5378), (5477- |
| | 5481), (7628-7632), (9271-9275), (9670-9674), (9684-9688), (11582-11586), |
| | (11624-11628), (11735-11739), (11955-11959), (12102-12106), (12428-12432), |
| | (12471-12475), (12698-12702) |
| G₅ | (4576-4580), (4912-4916), (6644-6648) |
| C₅ | (8757-8761), (12209-12213) |
| A₆ | (1862-1867), (2271-2276), (4299-4304), (4514-4519), (5081-5086), (6225-6230), |
| | (7695-7700), (7785-7790), (8328-8333), (9063-9068) |
| T₆ | (440-445), (2159-2164), (2476-2481), (3049-3054), (3415-3420), (3449-3454), |
| | (5511-5516), (8981-8986), (11779-11784), (11837-11842), (11864-11869), (12507- |
| | 12512), (13241-13246), (13275-13280) |
| G₆ | (6764-6769) |
| A₇ | (4898-4904), (5019-5025) |
| T₇ | (1116-1122), (3024-3030), (3265-3271), (5100-5106), (5145-5151), (5261-5267), |
| | (12818-12824) |
| T₈ | (2501-2508), (12462-12469), (13308-13315) |
| (AG)₄ | (1131-1138), (4552-4559) |
| (GA)₄ | (1396-1403), (2421-2428) |
| (TA)₄ | (3151-3158) |
| (AT)₄ | (4071-4078), (4082-4089) |
| (CT)₄ | (5304-5311), (9663-9670) |
| (CA)₄ | (8855-8862) |
| (GT)₅ | (7377-7386) |
| (AG)₆ | (4635-4646) |
| (GAG)₄ | (1000-1011) |
| (TGA)₄ | (2982-2993) |
| (GAA)₄ | (10141-10152) |
| (TTTAT)₃ | (9672-9683) |

The SSRs identified in the PDK_{—}30s65509694 scaffold (GenBank accession number: ACYX02084480-6649 nucleotides in length) and their respective locations in this scaffold are given in the following table:

| SSR | Positions occupées dans le scaffold PDK_30s65509694 |
|---|---|
| A₄ | (11-14), (53-56), (99-102), (135-138), (238-241), (352-355), (586-589), (633-636), |
| | (995-998), (1041-1044), (1203-1206), (1277-1280), (1543-1546), (1628-1631), |
| | (1815-1818), (1825-1828), (1944-1947), (2042-2045), (2081-2084), G4 (2138- |
| | 2141), (2181-2184), (2208-2211), (2213-2216), (2493-2496), (2649-2652), (2663- |
| | 2666), (2745-2748), (2830-2833), (3620-3623), (3826-3829), (3977-3980), (4613- |
| | 4616), (4697-4700), (4782-4785), (5174-5177), (5185-5188), (5281-5284), (5357- |
| | 5360), (5487-5490), (5605-5608), (5721-5724), (6082-6085), (6127-6130), |
| | (6505-6508) |
| T₄ | (90-93), (157-160), (451-454), (488-491), (497-500), (515-518), (744-747), (1047- |
| | 1050), (1479-1482), (2459-2462), (2501-2504), (2543-2546), (2591-2594), (2615- |
| | 2618), (2777-2780), (3134-3137), (3148-3151), (3352-3355), (4522-4525), (5269- |
| | 5272), (5472-5475), (5611-5614), (5643-5646), (6123-6126), (6137-6140), (6184- |
| | 6187), (6243-6246), (6295-6298), (6357-6360) |
| G₄ | (73-76), (1431-1434), (1954-1957), (5622-5625) |
| C₄ | (720-723), (3640-3643), (3926-3929), (4430-4433) |
| A₅ | (726-730), (1118-1122), (1226-1230), (1566-1570), (2000-2004), (1922-1926), |
| | (1988-1992), (2130-2134), (2374-2378), (2471-2475), (2525-2529), (2564-2568), |
| | (2606-2610), (2695-2699), (3247-3251), (3947-3951), (4383-4387), (4389-4393), |
| | (4570-4574), (4689-4693), (5179-5183), (5655-5659), (5732-5736), (6378-6382), |
| | (6488-6492) |
| T₅ | (84-88), (199-203), (527-531), (866-870), (2573-2577), (2763-2767), (3984-3988), |
| | (4531-4535), (5155-5159), (5249-5253), (5439-5443), (5750-5754), (5809-5813), |
| | (5858-5862), (5921-5925), (5927-5931), (6007-6011) |
| G₅ | (6-10), (1151-1155), (5096-5100) |
| C₅ | (22-26), (4832-4836), |
| A₆ | (416-421), (597-602), (1830-1835), (2009-2014), (2251-2256), (6062-6067), |
| T₆ | (359-364), (5889-5894) |
| T₇ | (5501-5507) |
| T8 | (2801-2808) |
| T₉ | (2753-2761) |
| T₁₀ | (2845-2854) |
| A₁₂ | (5457-5468) |
| (TC)₄ | (2993-3000), (3017-3024), (3095-3102), (6533-6540) |
| (AG)₄ | (3007-3014), (3111-3118) |
| (AT)₄ | (3671-3678) |
| (CT)₄ | (6166-6173) |
| (TCTT)₄ | (5979-5994) |
| (TTCC)₃ | (5994-6005) |

The SSRs identified in the PDK_{—}30s1095881 scaffold (GenBank accession number: GL745352-16579 nucleotides in length) and their respective locations in this scaffold are given in the following table:

| SSR | Positions occupées dans le scaffold PDK_30s1095881 |
|---|---|
| A₄ | (136-139), (236-239), (484-487), (548-551), (706-709), (968-971), (1371-1374), |
| | (1698-1701), (1723-1726), (1737-1740), (2037-2040), (2067-2070), (2196-2199), |
| | (2329-2332), (2405-2408), (2452-2455), (2463-2466), (2687-2690), (2797-2800), |
| | (2814-2817), (2879-2882), (3070-3073), (3174-3177), (3256-3259), (3320-3323), |
| | (3519-3522), (3892-3895), (3945-3948), (3959-3962), (4066-4069), (4075-4078), |
| | (4732-4735), (4974-4977), (5011-5014), (5210-5213), (5416-5419), (5633-5636), |
| | (5741-5744), (5802-5805), (5817-5820), (5986-5989), (6194-6197), (6629-6632), |
| | (6904-6907), (6924-6927), (6932-6935), (7075-7078), (7202-7205), (7411-7414), |
| | (7688-7691), (7799-7802), (7838-7841), (7878-7881), (8003-8006), (8185-8188), |
| | (8220-8223), (8331-8334), (8343-8346), (8451-8454), (8464-8467), (8547-8550), |
| | (8634-8637), (8654-8657), (8676-8679), (8776-8779), (8917-8920), (9098-9101), |
| | (9681-9684), (9733-9736), (9756-9759), (9784-9787), (9825-9828), (10163- |
| | 10166), (10484-10487), (10500-10503), (10569-10572), (10659-10662), (10679- |
| | 10682), (10916-10919), (10962-10965), (10989-10992), (11262-11265), (11683- |
| | 11686), (11691-11694), (11719-11722), (11773-11776), (12047-12050), (12426- |
| | 12429), (12532-12535), (12577-12580), (12630-12633), (12834-12837), (12977- |
| | 12980), (13051-13054), (13127-13130), (13219-13222), (13459-13462), (13533- |
| | 13536), (14086-14089), (14120-14123), T4 (14214-14217), (14275-14278), |
| | (14316-14319), (14375-14378), (14445-14448), (14499-14502), (14598-14601), |
| | (15686-15689), (15706-15709), (15750-15753), (15788-15791), (15830-15833), |
| | (15849-15852), (15872-15875), (16131-16134), (16167-16170), (16185-16188), |
| | (16487-16490) |
| T₄ | (283-286), (378-381), (403-406), (445-448), (569-572), (776-779), (846-849), |
| | (1097-1100), (1413-1416), (1460-1463), (1497-1500), (1596-1599), (1661-1664), |
| | (2229-2232), (2266-2269), (2276-2279), (2304-2307), (2525-2528), (2715-2718), |
| | (2804-2807), (2818-2821), (2936-2939), (3239-3242), (3360-3363), (3464-3467), |
| | (3675-3678), (3949-3952), (3977-3980), (4094-4097), (4230-4233), (4421-4424), |
| | (4724-4727), (4756-4759), (4828-4831), (5093-5096), (5568-5571), (5913-5916), |
| | (5992-5995), (6145-6148), (6285-6288), (6415-6418), (6844-6847), (7248-7251), |
| | (7483-7486), (7761-7764), (7808-7811), (7842-7845), (8059-8062), (8119-8122), |
| | (8258-8261), (8382-8385), (8403-8406), (8433-8436), (8510-8513), (8747-8750), |
| | (8831-8834), (8970-8973), (9052-9055), (9382-9385), (9390-9393), (9426-9429), |
| | (9438-9441), (9511-9514), (9812-9815), (9998-10001), (10226-10229), (10267- |
| | 10270), (10361-10364), (10665-10668), (10757-10760), (11086-11089), (11751- |
| | 11754), (11902-11905), (12709-12712), (12722-12725), (13196-13199), (13852- |
| | 13855), (14840-14843), (15624-15627), (15656-15659), (15693-15696), (16038- |
| | 16041), (16208-16211), (16266-16269), (16358-16361), (16397-16400) |
| G₄ | (168-171), (168-171), (178-181), (3590-3593), (3717-3720), (6432-6435), (10586- |
| | 10589), (10993-10996), (11446-11449), (14007-14010), (16213-16216), |
| C₄ | (95-98), (1166-1169), (1186-1189), (4314-4317), (4364-4367), (7887-7890), (9352- |
| | 9355), (10199-10202), (10508-10511), (10591-10594), (11698-11701), (11821- |
| | 11824), (12151-12154), (13485-13488), (14290-14293), (14535-14538), (14652- |
| | 14655), (14664-14667), (14879-14882), ), (14912-14915), (15080-15083) |
| A₅ | (611-615), (995-999), (1768-1772), (2212-2216), (2780-2784), (4269-4273), (4737- |
| | 4741), (4897-4901), (5500-5504), (5517-5521), (5662-5666), (5796-5800), (5898- |
| | 5902), (5949-5953), (6013-6017), (6634-6638), (6898-6902), (7005-7009), (8300- |
| | 8304), (8308-8312), (8348-8352), (8374-8378), (8479-8483), (8591-8595), (8713- |
| | 8717), (10285-10289), (11211-11215), (11414-11418), (12555-12559), (12773- |
| | 12777), (13654-13658), (14408-14412), (14668-14672), (14729-14733), (15639- |
| | 15643), (15962-15966), (16124-16128), (16174-16178), (16311-16315) |
| T₅ | (297-301), (1010-1014), (1074-1078), (1086-1090), (1234-1238), (1312-1316), |
| | (1396-1400), (1562-1566), (2081-2085), (2456-2460), (4781-4785), (6405-6409), |
| | (6607-6611), (6837-6841), (6975-6979), (7094-7098), (7269-7273), (8459-8463), |
| | (8601-8605), (8766-8770), (9255-9259), (9498-9502), (9703-9707), (9976-9980), |
| | (10966-10970), (11205-11209), (11341-11345), (14150-14154), (14883-14887) |
| G₅ | (10883-10887) |
| C₅ | (9377-9381), (9475-9479), (12181-12185) |
| A₆ | (1825-1830), (2886-2891), (3139-3144), (3148-3153), (8336-8341), (8978-8983), |
| | (10997-11002), (12547-12552), (12759-12764), (12959-12964) |
| T₆ | (589-594) (15678-15683), (15733-15738), (657-662), (5425-5430), (7148-7153), |
| | (9044-9049), (9305-9310), (14096-14101) |
| G₆ | (12076-12081) |
| A₇ | (721-727), (6425-6431), (6640-6646), (6828-6834), (7170-7176), (7372-7378), |
| | (7469-7475), (13787-13793), (14748-14754), (15697-15703) |
| T₇ | (1114-1120) |
| A₈ | (3059-3066), (6295-6302), (8363-8370) |
| T₈ | (386-393), (919-926), (3697-3704) |
| A₉ | (1130-1138) |
| T₁₀ | (6887-6896) |
| (GA)₄ | (4235-4242) |
| (AT)₄ | (5906-5913), (7916-7923), (16094-16101) |
| (CT)₄ | (9127-9134) |
| (AC)₅ | (13620-13629) |
| (TG)₆ | (10840-10851) |
| (CT)₆ | (12137-12148) |
| (TTA)₄ | (1119-1130) |
| (ATTT)₃ | (4156-4167) |
| (CTCC)₃ | (4324-4335) |
| (CAGG)₃ | (6157-6171) |
| (ACAT)₃ | (13628-13645) |

The SSRs identified in the PDK_{—}30s893961 scaffold (GenBank accession number: GL744384-20362 nucleotides in length) and their respective locations in this scaffold are given in the following table:

| SSR | Positions occupées dans le scaffold PDK_30s893961 |
|---|---|
| A₄ | (53-56), (233-236), (597-600), (768-771), (1050-1053), (1953-1956), (1983- |
| | 1986), (2314-2317), (2416-2419), (2880-2883), (3187-3190), (3193-3196), |
| | (3393-3396), (3790-3793), (3904-3907), (3932-3935), (4083-4086), (4331-4334), |
| | (4357-4360), (4374-4377), (4413-4416), (4430-4433), (4511-4514), (4573-4576), |
| | (4594-4597), (4672-4675), (4890-4893), (4995-4998), (5160-5163), (5482-5485), |
| | (5708-5711), (5846-5849), (5957-5960, (5987-5990), (6131-6134), (6275-6278), |
| | (6424-6427), (6608-6611), (6621-6624), (6630-6633), (6645-6648), (7040-7043), |
| | (7468-7471), (7560-7563), (7604-7607), (7879-7882), (7934-7937), (7950-7953), |
| | (8086-8089), (8091-8094), (8181-8184), (8415-8418), (8442-8445), (8567-8570), |
| | (9053-9056), (9060-9063), (9079-9082), (9107-9110), (9193-9196), (9340-9343), |
| | (9445-9448), (9464-9467), (9522-9525), (9534-9537), (9595-9598), (9615-9618), |
| | (9654-9657), (9745-9748), (9752-9755), (9853-9856), (9932-9935), (10267- |
| | 10270), (10300-10303), (10317-10320), (10380-10383), (10519-10522), (10532- |
| | 10535), (10655-10658), (10929-10932), (11615-11618), (11643-11646), (11831- |
| | 11834), (12205-12208), (12293-12296), (12451-12454), (12485-12488), (12576- |
| | 12579), (12959-12962), (13094-13097), (13136-13139), (13304-13307), (13337- |
| | 13340), (13414-13417), (13494-13497), (13564-13567), (14201-14204), (14741- |
| | 14744), (14754-14757), (14986-14989), (15080-15083), (15855-15858), (16024- |
| | 16027), (16064-16067), (16115-16118), (16145-16148), (16198-16201), (16228- |
| | 16231), (16309-16312), (16321-16324), (16373-16376), (16461-16464), (16929- |
| | 16932), 17252-17255), (17257-17260), (17280-17283), (17294-17297), (17449- |
| | 17452), (17894-17897), (18006-18009), (18043-18046), (18087-18090), (18266- |
| | 18269), (19005-19008), (19448-19451), (19513-19516), (19560-19563), |
| | (19861-19864), (20075-20078), (20312-20315) |
| T₄ | (371-374), (637-640), (1394-1397), (1921-1924), (2208-2211), (2869-2872), |
| | (3162-3165), (3331-3334), (3411-3414), (3772-3775), (4197-4200), (4219-4222), |
| | (4231-4234), (4288-4291), (4633-4636), (4711-4714), (4767-4770), (4848-4851), |
| | (4913-4916), (6062-6065), T4 (6229-6232), (6261-6264), (6375-6378), (6380- |
| | 6383), (6640-6643), (6801-6804), (7161-7164), (7451-7454), (7918-7921), |
| | (8137-8140), (8339-8342), (8382-8385), (8560-8563), (8815-8818), (8838-8841), |
| | (9234-9237), (9354-9357), (10542-10545), (10611-10614), (10744-10747), |
| | (10887-10890), (11168-11171), (11439-11442), (11467-11470), (11639-11642), |
| | (11881-11884), (12302-12305), (12950-12953), (13175-13178), (13260-13263), |
| | (13267-13270), (13389-13392), (13587-13590), (13832-13835), (13968-13971), |
| | (14498-14501), (15124-15127), (15278-15281), (15381-15384), (15594-15597), |
| | (15663-15666), (15700-15703), (15714-15717), (15877-15880), (15971-15974), |
| | (16280-16283), (16868-16871), (16923-16926), (17115-17118), (17304-17307), |
| | (17332-17335), (17415-17418), (17527-17530), (17555-17558), (17648-17651), |
| | (17825-17828), (18050-18053), (18273-18276), (18327-18330), (18337-18340), |
| | (18361-18364), (18483-18486), (18792-18795), (18808-18811), (18838-18841), |
| | (18939-18942), (18992-18995), (19501-19504), (20135-20138) |
| G₄ | (213-216), (649-652), (880-883), (1315-1318), (1376-1379), (1749-1752), (2134- |
| | 2137), (2377-2380), (2735-2738), (3070-3073), (3710-3713), (5170-5173), |
| | (5198-5201), (5260-5263), (5301-5304), (5325-5328), (5363-5366), (6102-6105), |
| | (6842-6845), (6982-6985), (6989-6992), (13143-13146), (16973-16976), (17084- |
| | 17087), (18918-18921), (18999-19002) |
| C₄ | (299-302), (633-636), (2057-2060), (2067-2070), (2173-2176), (2433-2436), |
| | (2591-2594), (2599-2602), (2627-2630), (2663-2666), (2766-2769), (2793-2796), |
| | (2819-2822), (3429-3432), (4107-4110), (4212-4215), (4807-4810), (8014-8017), |
| | (8721-8724), (8869-8872), (9038-9041), (11376-11379), (11929-11932), (12172- |
| | 12175), (12181-12184), (12628-12631), (13841-13844), (13886-13889), (14362- |
| | 14365), (15346-15349), (15817-15820), (16176-16179), (16215-16218), (16259- |
| | 16262), (16338-16341), (18195-18198), (18417-18420), (18746-18749), |
| | (19174-19177) |
| A₅ | (1916-1920), (4478-4482), (4537-4541), (4620-4624), (4746-4750), (5732-5736), |
| | (6205-6209), (6284-6288), (6324-6328), (7082-7086), (8473-8477), (8535-8539), |
| | (8579-8583), (8789-8793), (9257-9261), (9323-9327), (9329-9333), (9728-9732), |
| | (9963-9967), (10428-10432), (10819-10823), (11476-11480), (11600-11604), |
| | (12479-12483), (12603-12607), (13582-13586), (13592-13596), (15416-15420), |
| | (16032-16036), (16288-16292), (17103-17107), (17228-17232), (17271-17275), |
| | (17340-17344), (17482-17486), A5 (19490-19494), (19526-19530), |
| | (19644-19648), (19876-19880) |
| T₅ | (111-115), (393-397), (1636-1640), (2903-2907), (3127-3131), (3973-3977), |
| | (3979-3983), (4442-4446), (4931-4935), (5427-5431), (5587-5591), (6438-6442), |
| | (6551-6555), (7400-7404), (8607-8611), (9114-9118), (9120-9124), (10262- |
| | 10266), (10959-10963), (11368-11372), (12713-12717), (13638-13642), (13802- |
| | 13806), (14633-14637), (14990-14994), (15204-15208), (15373-15377), (15787- |
| | 15791), (15824-15828), (15888-15892), (16156-16160), (16361-16365), (17022- |
| | 17026), (17233-17237), (18347-18351), (18428-18432), (18862-18866), (18960- |
| | 18964), (18967-18971), (19238-19242), (19564-19568), (19588-19592), |
| | (19800-19804), (19974-19978) |
| G₅ | (2531-2535), (5279-5283), (5352-5356), (12501-12505), (16581-16585), |
| | (16749-16753) |
| C₅ | (404-408), (2604-2608), (12697-12701), (13397-13401), (14103-14107), |
| | (16055-16059) |
| A₆ | (2232-2237), (2301-2306), (3891-3896), (5877-5882), (6360-6365), (6944-6949), |
| | (9883-9888), (11008-11013), (11763-11768), (13126-13131), (14287-14292), |
| | (16129-16134), (16348-16353), (19927-19932) |
| T₆ | (3297-3302), (4649-4654), (12943-12948), (16239-16244), (17624-17629), |
| | (19414-19419) |
| G₆ | (5178-5183), (18098-18103), (18105-18110), |
| C₆ | (185-190), (185-190), (16805-16810) |
| A₇ | (5241-5247), (5738-5744), (9822-9828), (17677-17683), (19229-19235) |
| T₇ | (4800-4806), (11555-11561), (13357-13363) |
| A₈ | (2352-2359), (8550-8557), (8906-8913), (9044-9051), (9561-9568), (10934- |
| | 10941), ), (12566-12573), (13284-13291), (13429-13436) |
| T₈ | (5861-5868) |
| A₉ | (8916-8924), (9918-9926), (10220-10228) |
| A₁₃ | (5458-5470) |
| (TC)₄ | (43-50), (15574-15581), (18866-18873) |
| (GA)₄ | (2535-2542) |
| (CT)₄ | (3794-3801), (6515-6522) |
| (TC)₉ | (5019-5036) |
| (AG)₄ | (5163-5170), (12488-12495) |
| (AT)₄ | (6365-6372) |
| (TC)₅ | (12703-12712), (16835-16844) |
| (CT)₅ | (18884-18893) |
| (AT)₆ | (9361-9372), (13756-13767) |
| (TA)₆ | (15918-15929) |
| (AG)₁₆ | (5210-5241) |
| (GA)₇ | (16473-16486) |
| (GA)₈ | (17063-17078) |
| (TA)₉ | (17741-17758) |
| (ACC)₄ | (14093-14104) |
| (AAT)₄ | (17591-17602) |
| (GAAA)₃ | (1974-1985) |
| (CCTC)₃ | (12682-12693) |
| (ACCA)₃ | (14130-14141) |
| (GGAT)₃ | (14425-14436) |
| (AAGA)₄ | (2509-2524) |
| (GAGAGG)3 | (12507-12524) |

The SSRs identified in the PDK_{—}30s1023161 scaffold (GenBank accession number: GL745445-16271 nucleotides in length) and their respective locations in this scaffold are given in the following table:

| SSR | Positions occupées dans le scaffold PDK_30s1023161 |
|---|---|
| A₄ | (495-498), (551-554), (785-788), (808-811), (871-874), (1432-1435), (3274-3277), |
| | (3428-3431), (3482-3485), (4381-4384), (4535-4538), (4698-4701), (4703-4706), |
| | (4756-4759), (4973-4976), (5017-5020), (5048-5051), (5750-5753), (5870-5873), |
| | (5909-5912), (5920-5923), (5966-5969), (5997-6000), (6025-6028), (6057-6060), |
| | (6380-6383), (6575-6578), (6818-6821), (7233-7236), (7633-7636), (7790-7793), |
| | (8115-8118), (8169-8172), (8289-8292), (8330-8333), (8540-8543), (8727-8730), |
| | (13265-13268), (14509-14512), (14630-14633), (16001-16004) |
| T₄ | (190-193), (285-288), (484-487), (513-516), (2196-2199), (2310-2313), (2317- |
| | 2320), (2411-2414), (2450-2453), (3008-3011), (3122-3125), (3129-3132), (3223- |
| | 3226), (3262-3265), (3326-3329), (3439-3442), (3494-3497), (3981-3984), (4115- |
| | 4118), (4229-4232), (4236-4239), (4330-4333), (4369-4372), (4433-4436), (4546- |
| | 4549), (4726-4729), (4737-4740), (4742-4745), (4861-4864), (4884-4887), (5010- |
| | 5013), (5204-5207), (5224-5227), (5288-5291), (5369-5372), (5406-5409), (5578- |
| | 5581), (5657-5660), (5682-5685), (5850-5853), (5905-5908), (5924-5927), (6015- |
| | 6018), (6098-6101), (6105-6108), (6146-6149), (6206-6209), (6310-6313), (6470- |
| | 6473), (6670-6673), (6720-6723), (6762-6765), (6861-6864), (7036-7039,) (7062- |
| | 7065), (7078-7081), (7554-7557), (7719-7722), (7975-7978), (7993-7996), (8017- |
| | 8020), (8309-8312), (8317-8320), (8483-8486), (8759-8762), (9580-9583), (12287- |
| | 12290), (12458-12461), (12944-12947), (13139-13142), (13163-13166), (13277- |
| | 13280), (13288-13291), (13455-13458), (13684-13687), (13787-13790), (13990- |
| | 13993), (14153-14156), (14171-14174), (14285-14288), (14295-14298), (14374- |
| | 14377), (14488-14491), (14598-14601), (14798-14801), (15589-15592), (15647- |
| | 15650), (16240-16243), (16264-16267) |
| G₄ | (358-361), (625-628), (1221-1224), (1882-1885), (1962-1965), (2035-2038), (2045- |
| | 2048), (2694-2697), (2774-2777), (2847-2850), (2857-2860), (3410-3413), (3801- |
| | 3804), (3881-3884), (3954-3957), (3964-3967), (4517-4520), (4600-4603), (7245- |
| | 7248), (8694-8697), (8879-8882), (9020-9023), (9320-9323), (9475-9478), (9641- |
| | 9644), (9756-9759), (9763-9766), (9808-9811), (9833-9836), (10008-10011), |
| | (12167-12170), (12340-12343), (12968-12971), (13034-13037), (14484-14487), |
| | (14674-14677), (15207-15210), (15278-15281), (15300-15303), (15450-15453), |
| | (15565-15568), (15757-15760), (15827-15830), (15919-15922), (16099-16102) |
| C₄ | (382-385), (1117-1120), (1945-1948), (2757-2760), (3864-3867), (5693-5696), |
| | (7336-7339), (9257-9260), (9773-9776), (10066-10069), (10140-10143), (13451- |
| | 13454), (14406-14409), (14748-14751), (15225-15228), (15518-15521) |
| A₅ | (5383-5387), (6486-6490), (14501-14505) |
| T₅ | (142-146), (179-183), (527-531), (968-972), (2062-2066), (2226-2230), (2429- |
| | 2433), (2874-2878), (3038-3042), (3241-3245), (3281-3285), (4145-4149), (4348- |
| | 4352), (4388-4392), (4490-4494), (5070-5074), (5537-5541), (6220-6224), (6435- |
| | 6439), (6538-6542), (6822-6826), (6950-6954), (7692-7696), (8038-8042), (8587- |
| | 8591), (8681-8685), (8764-8768), (8783-8787), (12647-12651), (13366-13370), |
| | (13706-13710), (13723-13727), (14347-14351) |
| G₅ | (5303-5307), (9825-9829), (9951-9955), (15264-15268), (15696-15700), |
| | (15940-15944) |
| C₅ | (2154-2158), (2966-2970), (4073-4077), (12355-12359) |
| A₆ | (998-1003), (1009-1014), (7816-7821) |
| T₆ | (2108-2113), (2920-2925), (3363-3368), (4027-4032), (4470-4475), (4776-4781), |
| | (5953-5958), (6459-6464), (13983-13988) |
| G₆ | (1616-1621), (3517-3522), (9590-9595), (9728-9733), (15144-15149), |
| | (15558-15563) |
| A₇ | (423-429), (4648-4654), (5840-5846) |
| T₇ | (275-281), (6687-6693), (14127-14133), (14562-14568) |
| A₈ | (5706-5713) |
| T₈ | (4637-4644), (5375-5382) |
| A₉ | (4589-4597), (6068-6076) |
| G₉ | (15021-15029) |
| (CT)₄ | (14117-14124) |
| (CA)₅ | (7905-7914) |
| (GA)₅ | (8818-8827) |
| (GA)₇ | (8839-8852) |

The SSRs identified in the PDK_{—}30s675061 scaffold (GenBank accession number: GL749237-7694 nucleotides in length) and their respective locations in this scaffold are given in the following table:

| SSR | Positions occupées dans le scaffold PDK_30s675061 |
|---|---|
| A₄ | (266-269), (607-610), (622-625), (667-670), 729-732), (832-835), (900-903), (986- |
| | 989), (1097-1100), (1125-1128), (1176-1179), (1434-1437), (1442-1445), (1562- |
| | 1565), (1686-1689), (1718-1721), (1800-1803), (1891-1894), (1939-1942), (2097- |
| | 2100), (2585-2588), (2668-2671), (2702-2705), (3074-3077), (3174-3177), (3392- |
| | 3395), (3477-3480), (3538-3541), (3559-3562), (4297-4300), (4306-4309), (4344- |
| | 4347), (4850-4853), (5372-5375), (5695-5698), (6240-6243), (6564-6567), |
| | (6754-6757), (7625-7628) |
| T₄ | (1207-1210), (1306-1309), (1618-1621), (1709-1712), (1740-1743), (1787-1790), |
| | (2059-2062), (2141-2144), (2467-2470), (2548-2551), (2902-2905), (2925-2928), |
| | (3122-3125), (3162-3165), (3346-3349), (3586-3589), (3651-3654), (3768-3771), |
| | (3790-3793), (3946-3949), (3986-3989), (4114-4117), (4235-4238), (4332-4335), |
| | (4451-4454), (4492-4495), (4595-4598), (4761-4764), (5428-5431), (5877-5880), |
| | (6042-6045), (6157-6160), (6368-6371), (6435-6438), (6468-6471), (6594-6597), |
| | (6599-6602), (6623-6626), (6678-6681), (7020-7023), (7136-7139), (7176-7179), |
| | (7253-7256), (7268-7271), (7279-7282), (7662-7665), (7668-7671), (7690-7693) |
| G₄ | (550-553), (3227-3230), (4105-4108), (6933-6936), (6963-6966), (6984-6987), |
| | (7026-7029), (7079-7082), (7142-7145), (7195-7198), (7216-7219), (7287-7290), |
| | (7308-7311), (7410-7413), (7503-7506) |
| C₄ | (2018-2021), (2237-2240), (2502-2505), (3772-3775), (4386-4389), (4923-4926) |
| | (6292-6295), (6324-6327), (6782-6785) |
| A₅ | (367-371), (1090-1094), (1422-1426), (1550-1554), (1865-1869), (1944-1948), |
| | (2027-2031), (2347-2351), (2740-2744), (3141-3145), (3804-3808), (3914-3918), |
| | (4513-4517), (4813-4817), (5322-5326), (6051-6055), (6387-6391), (6420-6424), |
| | (6867-6871), (7563-7567) |
| T₅ | (1161-1165), (1182-1186), (1217-1221), (2563-2567), (2571-2575), (3055-3059), |
| | (3300-3304), (3313-3317), (3325-3329), (3396-3400), (4482-4486), (5744-5748), |
| | (5799-5803), (5864-5868), (5975-5979), (6103-6107), (6405-6409), (6726-6730) |
| G₅ | (1427-1431), (1518-1522), (4808-4812) |
| C₅ | (2090-2094), (2277-2281), (2319-2323), (5596-5600) |
| A₆ | (701-706), (1491-1496), (1753-1758), (1874-1879), (2732-2737), (3102-3107), |
| | (5758-5763), (6511-6516), |
| T₆ | (1199-1204), (4290-4295), (4437-4442), (5870-5875) |
| G₆ | (5384-5389) |
| A₇ | (2610-2616) |
| T₇ | (1722-1728), (4457-4463), (4617-4623), (5815-5821), (6977-6983), (7522-7528) |
| C₇ | (3446-3452) |
| T₈ | (3423-3430) |
| A₉ | (1567-1575) |
| G₁₀ | (6138-6147) |
| (GA)₄ | (385-392) |
| (CT)₅ | (1044-1053) |
| (TG)₆ | (6195-6206) |

The SSRs identified in the PDK_{—}30s925161 scaffold (GenBank accession number: GL742468-31314 nucleotides in length) and their respective locations in this scaffold are given in the following table:

| SSR | Positions occupées dans le scaffold PDK_30s925161 |
|---|---|
| A₄ | (256-259), (276-279), (588-591), (712-715), (730-733), (949-952), (1087-1090), |
| | (1108-1111), (1160-1163), (1323-1326), (1401-1404), A4 (1699-1702), (2079- |
| | 2082), ), (2279-2282), (2309-2312), (3352-3355), (3469-3472), (3704-3707), |
| | (3711-3714), (3800-3803), (3857-3860), (3933-3936), (3994-3997), (4008-4011), |
| | (4149-4152), (4973-4976), (5064-5067), (5439-5442), (5811-5814), (6397-6400), |
| | (6511-6514), (6586-6589), (6848-6851), (6889-6892), (6997-7000), (7469-7472), |
| | (7519-7522), (7531-7534), (7538-7541), (7597-7600), (7758-7761), (8118-8121), |
| | (8489-8492), (8664-8667), (8673-8676), (8712-8715), (8753-8756), (8779-8782), |
| | (8825-8828), (9156-9159), (9353-9356), (9541-9544), (9600-9603), (9629-9632), |
| | (10316-10319), (11218-11221), (11369-11372), (11601-11604), (11871-11874), |
| | (12538-12541), (12615-12618), (12682-12685), (12927-12930), (13327-13330), |
| | (13339-13342), (13357-13360), (13441-13444), (13534-13537), (13577-13580), |
| | (13593-13596), (13860-13863), (13977-13980), (13984-13987), (14061-14064), |
| | (14101-14104), (14451-14454), (14544-14547), (14659-14662), (14875-14878), |
| | (14925-14928), (15121-15124), (15141-15144), (15640-15643), (15679-15682), |
| | (15886-15889), (15906-15909), (16120-16123), (16247-16250), (16326-16329), |
| | (16394-16397), (16423-16426), (16773-16776), (16817-16820), (16973-16976), |
| | (17118-17121), (19110-19113), (19430-19433), (19447-19450), (19642-19645), |
| | (19758-19761), (19810-19813), (20123-20126), (20372-20375), (20770-20773), |
| | (20809-20812), (20831-20834), (20864-20867), (21233-21236), (21582-21585), |
| | (21820-21823), (21964-21967), (22080-22083), (22194-22197), (22496-22499), |
| | (22725-22728), (22856-22859), (22947-22950), (23087-23090), (23098-23101), |
| | (23181-23184), (23258-23261), (23342-23345), (23531-23534), (23580-23583), |
| | (25103-25106), (25193-25196), (25198-25201), (25444-25447), (25588-25591), |
| | (25682-25685), (25730-25733), (25782-25785), (26262-26265), (26306-26309), |
| | (26476-26479), (26557-26560), (26690-26693), (26979-26982), (27062-27065), |
| | (27097-27100), (27128-27131), (27525-27528), (27658-27661), (27722-27725), |
| | (27778-27781), (27794-27797), (28027-28030), (28064-28067), (28312-28315), |
| | (28447-28450), (28471-28474), (28793-28796), (29809-29812), (31227-31230) |
| T₄ | (78-81), (327-330), (433-436), (549-552), (1097-1100), (1527-1530), (1584-1587), |
| | (1672-1675), (1892-1895), (2162-2165), (2202-2205), (2463-2466), (2483-2486), |
| | (2680-2683), (2687-2690), (2908-2911), (2977-2980), (3017-3020), (3046-3049), |
| | (3058-3061), (3144-3147), (3217-3220), (3264-3267), (3344-3347), (3606-3609), |
| | (3623-3626), (3672-3675), (3686-3689), (3835-3838), (4373-4376), (4394-4397), |
| | (4554-4557), (4699-4702), (4732-4735), (4902-4905), (5078-5081), (5136-5139), |
| | (5203-5206), (5271-5274), (5423-5426), (5514-5517), (5677-5680), (5682-5685), |
| | (5715-5718), (6001-6004), (6074-6077), (6079-6082), (6148-6151), (6466-6469), |
| | (6881-6884), (6950-6953), (7011-7014), (7019-7022), (7027-7030), (7061-7064), |
| | (7527-7530), (7586-7589), (7678-7681), (7740-7743), (7763-7766), (7808-7811), |
| | (8007-8010), (8130-8133), (8192-8195), (8399-8402), (8959-8962), (9026-9029), |
| | (9545-9548), (9625-9628), (9755-9758), (9832-9835), (9852-9855), (9964-9967), |
| | (10720-10723), (11155-11158), (11530-11533), (12113-12116), (12125-12128), |
| | (12275-12278), (12304-12307), (12380-12383), (12564-12567), (12677-12680), |
| | (12726-12729), (12771-12774), (12777-12780), (13211-13214), (13281-13284), |
| | (13311-13314), (13524-13527), (13529-13532), (14016-14019), (14386-14389), |
| | (14396-14399), (14532-14535), (14608-14611), (14629-14632), (14780-14783), |
| | (14885-14888), (15207-15210), (15744-15747), (16108-16111), (16373-16376), |
| | (16419-16422), (16585-16588), (16936-16939), (17006-17009), (17151-17154), |
| | (17232-17235), (17626-17629), (18098-18101), (18106-18109), (18120-18123), |
| | (18487-18490), (18509-18512), (18519-18522), (18679-18682), (19190-19193), |
| | (19805-19808), (19884-19887), (19933-19936), (19988-19991), (20226-20229), |
| | (20293-20296), (20340-20343), (20462-20465), (20737-20740), (20886-20889), |
| | (20935-20938), (20975-20978), (21036-21039), (21475-21478), (21506-21509), |
| | (21520-21523), (21541-21544), (22062-22065), (22084-22087), (22131-22134), |
| | (22214-22217), (22220-22223), (22258-22261), (22285-22288), (22334-22337), |
| | (22834-22837), (22875-22878), (23373-23376), (23574-23577), (23771-23774), |
| | (23904-23907), (25301-25304), (25324-25327), (25425-25428), (25492-25495), |
| | (25534-25537), (25569-25572), (25636-25639), (25678-25681), (25711-25714), |
| | (25778-25781), (25927-25930), (26042-26045), (26094-26097), (26113-26116), |
| | (26513-26516), (26531-26534), (26701-26704), (26728-26731), (26769-26772), |
| | (26864-26867), (26881-26884), (26983-26986), (27008-27011), (27192-27195), |
| | (27198-27201), (27272-27275), (27343-27346), (27489-27492), (28019-28022), |
| | (28129-28132), (28179-28182), (28184-28187), (28375-28378), (28384-28387), |
| | (28481-28484), (28532-28535), (28654-28657), (28731-28734), (28783-28786), |
| | (28788-28791), (28833-28836), (29043-29046), (29495-29498), (30948-30951), |
| | (30965-30968), (31243-31246) |
| G₄ | (854-857), (1211-1214), (6529-6532), (7181-7184), (11419-11422), (11758- |
| | 11761), (12964-12967), (14447-14450), (16511-16514), (16547-16550), (16932- |
| | 16935), (16960-16963), (17726-17729), (17841-17844), (17917-17920), (17956- |
| | 17959), (18003-18006), (18223-18226), (18367-18370), (18571-18574), (19462- |
| | 19465), (19703-19706), (21089-21092), (25359-25362), (26750-26753), (28138- |
| | 28141), (30612-30615), (30662-30665) |
| C₄ | (542-545), (954-957), (2121-2124), (4086-4089), (7882-7885), (8481-8484), (8863- |
| | 8866), (8890-8893), (8953-8956), (10939-10942), (10975-10978), (11202-11205), |
| | (11226-11229), (13456-13459), (13598-13601), (14666-14669), (14751-14754), |
| | (14832-14835), (15015-15018), (15597-15600), (15780-15783), (16891-16894), |
| | (17028-17031), (17662-17665), (17832-17835), (18017-18020), (18208-18211), |
| | (18596-18599), (18942-18945), (20063-20066), (25209-25212), (25227- 25230), |
| | (26081-26084), (29589-29592), (29946-29949), (29955-29958), (30957-30960) |
| A₅ | (268-272), (528-532), (706-710), (717-721), (750-754), (2000-2004), (4032-4036), |
| | (5733-5737), (6244-6248), (6989-6993), (7066-7070), (7543-7547), (7961-7965), |
| | (9358-9362), (9573-9577), (9970-9974), (10043-10047), (10562-10566), (12011- |
| | 12015), (12941-12945), (13306-13310), (13446-13450), (13562-13566), (14119- |
| | 14123), (14482-14486), (14700-14704), (14854-14858), (14880-14884), (14914- |
| | 14918), (15619-15623), (15664-15668), (16155-16159), (16615-16619), (16865- |
| | 16869), (18736-18740), (22433-22437), (22588-22592), (22607-22611), (22782- |
| | 22786), (23351-23355), (23437-23441), (23793-23797), (25090-25094), (26335- |
| | 26339), (27788-27792), (27806-27810), (28290-28294), (28455-28459), (28560- |
| | 28564), (30000-30004), (31214-31218) |
| T₅ | (2216-2220), (2604-2608), (2656-2660), (2818-2822), (2874-2878), (2895-2899), |
| | (2959-2963), (3498-3502), (3596-3600), (3760-3764), (4341-4345), (4773-4777), |
| | (4787-4791), (4820-4824), (6976-6980), (7463-7467), (7591-7595), (7783-7787), |
| | (8038-8042), (8721-8725), (9528-9532), (9616-9620), (12189-12193), (12456- |
| | 12460), (12712-12716), (12784-12788), (13253-13257), (13702-13706), (13810- |
| | 13814), (14053-14057), (14186-14190), (16269-16273), (16441-16445), (16531- |
| | 16535), (16672-16676), (16983-16987), (17475-17479), (19607-19611), (19904- |
| | 19908), (20112-20116), (20796-20800), (20843-20847), (21288-21292), (21419- |
| | 21423), (21604-21608), (22021-22025), (22806-22810), (22887-22891), (23212- |
| | 23216), (23502-23506), (23909-23913), (26070-26074), (26177-26181), (26391- |
| | 26395), (26442-26446), (27045-27049), (28618-28622), (28996-29000), (29377- |
| | 29381), (30942-30946), (31121-31125) |
| G₅ | (743-747), (17783-17787), (18958-18962), (19025-19029), (22655-22659), (25269- |
| | 25273), (30198-30202), (31004-31008) |
| C₅ | (2255-2259), (2343-2347), (7227-7231), (8606-8610), (8802-8806), (8975-8979), |
| | (25035-25039), (27292-27296) |
| A₆ | (4460-4465), (4963-4968), (5552-5557), (8143-8148), (8202-8207), (11230- |
| | 11235), (13139-13144), (14462-14467), (21441-21446), (25109-25114), (25476- |
| | 25481), (25620-25625), (25762-25767), (27799-27804), (28212-28217), |
| | (29446-29451) |
| T₆ | (2742-2747), (5294-5299), (5775-5780), (6967-6972), (7665-7670), (8122-8127), |
| | (9660-9665), (9695-9700), (9926-9931), (12136-12141), (13347-13352), (15250- |
| | 15255), (15390-15395), (19233-19238), (21454-21459), (21860-21865), (22251- |
| | 22256), (22957-22962), (28259-28264) |
| G₆ | (1572-1577), (2928-2933), (7729-7734) |
| C₆ | (2087-2092), (9324-9329), (29858-29863) |
| A₇ | (3534-3540), (7445-7451), (15918-15924), (22207-22213), (26893-26899), (26951- |
| | 26957), (27704-27710), (27733-27739), (31156-31162), (31286-31292) |
| T₇ | (4251-4257), (7500-7506), (7565-7571), (28246-28252), (29092-29098), |
| | (31140-31146) |
| C₇ | (17687-17693) |
| A₈ | (2410-2417), (15153-15160), (16193-16200), (18724-18731), (26344-26351), |
| | (23139-23146), (29499-29506) |
| T₈ | (2400-2407), (23640-23647), (29559-29566) |
| T₉ | (5455-5463), (6085-6093), (29231-29239), (31167-31175) |
| A₁₀ | (20754-20763) |
| T₁₀ | (18125-18134), (28343-28352), (29533-29542) |
| A₁₂ | (29219-29230) |
| T₁₈ | (22927-22944), (29410-29427) |
| (CT)₄ | (832-839), (17585-17592), (25173-25180) |
| (TA)₄ | (1069-1076) |
| (GA)₄ | (2384-2391), (2585-2592), (6693-6700) |
| (AT)₄ | (12855-12862), (23040-23047) |
| (TA)₅ | (12716-12725) |
| (CT)₅ | (17672-17681) |
| (AT) ₅ | (26501-26510) |
| (AT)₆ | (26642-26653) |
| (AT)₂₅ | (26899-26948) |
| (TAC)₄ | (29292-29303) |
| (TAC)₅ | (29328-29342) |
| (TAT)₆ | (29343-29360), |
| (ATT)₁₄ | (29253-29293) |
| (TTTA)₃ | (22816-22827) |
| (TACA)₄ | (8550-8565) |
| (TTTA)₅ | (4374-4393) |
| (GCTAG)₄ | (14332-14351) |

The SSRs identified in the PDK_{—}30s680001 scaffold (GenBank accession number: GL745189-17202 nucleotides in length) and their respective locations in this scaffold are given in the following table:

| SSR | Positions occupées dans le scaffold PDK_30s680001 |
|---|---|
| A₄ | (857-860), (1082-1085), (1836-1839), (2324-2327), (3008-3011), (3250-3253), |
| | (3292-3295), (3601-3604), (3862-3865), (3977-3980), (4570-4573), (4864-4867), |
| | (4924-4927), (4950-4954), (4975-4978), (5341-5344), (6953-6956), (8187-8190), |
| | (8198-8201), (8215-8218), (8326-8329), (8389-8392), (8555-8558), (8639-8642), |
| | (8654-8657), (8716-8719), (8728-8731) (8773-8776), (9111-9114), (9395-9398), |
| | (9724-9727), (9757-9760), (9997-10000), (10889-10892), (11060-11063), (11162- |
| | 11165), (11416-11419), (11469-11472), (11527-11530), (12532-12535), (12537- |
| | 12540), (12859-12862), (12864-12867), (12983-12986), (13026-13029), (13274- |
| | 13277), (13479-13482), (13495-13498), (13553-13556), (13695-13698), (13948- |
| | 13951), (13990-13993), (14034-14037), (14205-14208), (14896-14899), (14970- |
| | 14973), (15080-15083), (15220-15223), (15568-15571), (15629-15632), |
| | (15792-15795), (16246-16249) |
| T₄ | (280-283), (440-443), (445-448), (668-671), (697-700), (788-791), (839-842), |
| | (1274-1277), (1312-1315), (1796-1799), (1851-1854), (2270-2273), (2334-2337), |
| | (2566-2569), (2727-2730), (3042-3045), (3070-3073), (3511-3514), (3704-3707), |
| | (3894-3897), (4112-4115), (4159-4162), (4195-4198), (4485-4488), (4605-4608), |
| | (4667-4670), (4874-4877), (4910-4913), (5202-5205), (5797-5800), (5865-5868), |
| | (6203-6206), (7273-7276), (7377-7380), (7609-7612), (7661-7664), (7669-7672), |
| | (7783-7786), (7960-7963), (8101-8104), (8107-8110), (8138-8141), (8162-8165), |
| | (8287-8290), (8351-8354), (8447-8450), (8674-8677), (8724-8727), (9069-9072), |
| | (9226-9229), (9556-9559), (9563-9566), (9623-9626), (9712-9715), (9837-9840), |
| | (9844-9847), (9946-9949), (10206-10209), (10310-10313), (10480-10483), (10485- |
| | 10488), (10614-10617), (10816-10819), (12184-12187), (12476-12479), (12673- |
| | 12676), (12834-12837), (12971-12974), (13295-13298), (13489-13492), (13823- |
| | 13826), (14450-14453), (14720-14723), (14863-14866), (15166-15169), (15249- |
| | 15252), (15385-15388), (15440-15443), (15511-15514), (15592-15595), (15650- |
| | 15653), (15692-15695), (15776-15779), (15858-15861), (15864-15867), (15886- |
| | 15889), (15892-15895), (16109-16112), (16314-16317), (16516-16519), |
| | (16991-16994) |
| G₄ | (114-117), (3303-3306), (3738-3741), (5377-5380), (6254-6257), (6291-6294), |
| | (6305-6308), (6322-6325), (6596-6599), (6705-6708), (7056-7059), (7094-7097), |
| | (7123-7126), (7216-7219), (7455-7458), (8435-8438), (10837-10840), (11499- |
| | 11502), (13728-13731), (14870-14873), (16538-16541), (16672-16675), (16817- |
| | 16820), (16836-16839), (16953-16956) |
| C₄ | (26-30), (45-48), (175-178), (192-195), (358-361), (406-409), (453-456), (5271- |
| | 5274), (5545-5548), (5871-5874), (5901-5904), (5948-5951), (6718-6721), (6781- |
| | 6784), (6921-6924), (7012-7015), (7027-7030), (7114-7117), (7201-7204), (9125- |
| | 9128), (9139-9142), (10029-10032), (10074-10077), (11898-11901), (12169- |
| | 12172), (12515-12518), (16678-16681), (17051-17054) |
| A₅ | (816-820), (1346-1350), (2070-2074), (2375-2379), (3149-3153), (3333-3337), |
| | (4073-4077), (4279-4283), (7238-7242), (7996-8000), (10398-10402), (10871- |
| | 10875), (11545-11549), (13470-13474), (14397-14401), (14979-14983), |
| | (15375-15379) |
| T₅ | (925-929), (989-993), (1121-1125), (1447-1451), (1580-1584), (1778-1782), (2913- |
| | 2917), (3048-3052), (3446-3450), (3474-3478), (3715-3719), (4505-4509), (5912- |
| | 5916), (8119-8123), (8456-8460), (8837-8841), (9146-9150), (9656-9660), (9861- |
| | 9865), (9992-9996), (10133-10137), (11492-11496), (12570-12574), (13143- |
| | 13147), (13522-13526), (14064-14068), (14363-14367), (15349-15353), (15412- |
| | 15416), (15620-15624), (15635-15639), (15834-15838), (15879-15883), (16013- |
| | 16017), (16029-16033), (16035-16039), (16371-16375) |
| G₅ | (2380-2384), (2716-2720), (3936-3940), (5262-5266), (5749-5753), (6517-6521), |
| | (6537-6541), (6990-6994), (7461-7465), (7471-7475), (9157-9161) |
| C₅ | (215-219), (420-424), (5393-5397), (7128-7132) |
| A₆ | (1221-1226), (4425-4430), (7229-7234) |
| T₆ | (1898-1903), (3542-3547), (4727-4732), (5059-5064), (9100-9105), (10392- |
| | 10397), (15263-15268), (16335-16340) |
| G₆ | (7621-7626) |
| C₆ | (160-165), (4702-4707) |
| A₇ | (7726-7732), (8002-8008), (12486-12492) |
| T₇ | (502-508), (1936-1942), (2029-2035), (5400-5406), (7886-7892), (12522-12528) |
| G₇ | (3942-3948), (6586-6592) |
| A₈ | (7734-7741), (12454-12461), (12542-12549), (14812-14819) |
| T₈ | (6654-6661), (12706-12713), (13903-13910), (14749-14756), (15516-15523) |
| A₉ | (7716-7724) |
| T₉ | (7641-7649) |
| A₁₃ | (4395-4407) |
| T₃₁ | (3905-3935) |
| (TC)₄ | (4368-4375) |
| (AT)₄ | (4888-4895), (9664-9671), (10795-10802), (13047-13054) |
| (GT)₄ | (7517-7524) |
| (AG)₄ | (9059-9066), (12492-12499) |
| (CT)₁₆ | (10276-10307) |
| (TA)₆ | (12408-12419) |
| (ACA)₄ | (3277-3288) |
| (GAAAA)₅ | (12531-12545) |

The SSRs identified in the PDK_{—}30s1004611 scaffold (GenBank accession number: GL751896-4471 nucleotides in length) and their respective locations in this scaffold are given in the following table:

| SSR | Positions occupées dans le scaffold PDK_30s1004611 |
|---|---|
| A₄ | (78-81), (1043-1046), (1473-1476), (1561-1564), (1649-1652), (1694-1697), (1953- |
| | 1956), (2019-2022), (2059-2062), (2071-2074), (2112-2115), (2204-2207), (2239- |
| | 2242), (2375-2378), (2476-2479), (2533-2536), (2538-2541), (2679-2682), (2709- |
| | 2712), (2770-2773), (2840-2843), (3038-3041), (3060-3063), (3386-3389), (3620- |
| | 3623), (3652-3655), (4214-4217), (4305-4308) |
| T₄ | (92-295), (704-707), (749-752), (789-792), (1690-1693), (2015-2018), (2046-2049), |
| | (2094-2097), (2217-2220), (2370-2373), (3317-3320), (4292-4295), (4337-4340), |
| | (4391-4394) |
| G₄ | (150-153), (392-395), (431-434), (592-595), (1090-1093), (2613-2616), (2651- |
| | 2654), (3044-3047), (3205-3208), (3313-3316), (3817-3820), (4465-4468) |
| C₄ | (775-778), (3541-3544), (3573-3576) |
| A₅ | (1095-1099), (1661-1665), (1726-1730), (1784-1788), (2256-2260), (2356-2360), |
| | (2513-2517), (2519-2523), (3066-3070) |
| T₅ | (823-827), (1175-1179), (1389-1393), (1708-1712), (1963-1967), (1066-1070) |
| G₅ | (1138-1142), (2882-2886), (4429-4433) |
| C₅ | (2271-2275), (3417-3421), (3629-3633), (4061-4065) |
| A₆ | (1550-1555), (1898-1903), (2865-2870), (3172-3177), (4272-4277) |
| G₆ | (4007-4012), (4018-4023) |
| T₇ | (4000-4006) |
| G₇ | (4000-4006) |
| A₈ | (1074-1080) |
| T₈ | (2660-2667) |
| A₁₀ | (1353-1362) |
| (TA)₂ | (1-8) |
| (GA)₂ | (645-652) |
| (TAAAT)₃ | (1440-1454) |

The SSRs identified in the PDK_{—}30s717571 scaffold (GenBank accession number: GL743202-26372 nucleotides in length) and their respective locations in this scaffold are given in the following table:

| SSR | Positions occupées dans le scaffold PDK_30s717571 |
|---|---|
| A₄ | (487-490), (962-965), (2299-2302), (2341-2344), (2711-2714), (2877- |
| | 2880), (2935-2938), (2946-2949), (2991-2994), (3191-3194), (3442-3445), (3607- |
| | 3610), (4206-4209), (4234-4237), (4614-4617), (4741-4744), (5337-5340), |
| | (5434-5437), (5458-5461), (5669-5672), (5691-5694), (5759-5762), (6046-6049), |
| | (6336-6339), (6437-6440), (6448-6451), (6558-6561), (6578-6581), (6661-6664), |
| | (6667-6670), (6795-6798), (6807-6810), (7050-7053), (6887-6890), (6896-6899), |
| | (7689-7692), (7825-7828), (7848-7851), (7872-7875), (7888-7891), (7898-7901), |
| | (7958-7961), (8056-8059), (8082-8085), (8355-8358), (8438-8441), (8804-8807), |
| | (8818-8821), (8929-8932), (9041-9044), (9259-9262), (9301-9304), (9351-9354), |
| | (9616-9619), (9621-9624), (9626-9629), (9637-9640), (9719-9722), (9742-9745), |
| | (10450-10453), (10627-10630), (10912-10915), (11053-11056), (11066-11069), |
| | (10678-10681), (11220-11223), (11343-11346), (11375-11378), (11410-11413), |
| | (11774-11777), (11797-11800), (11935-11938), (11957-11960), (12134-12137), |
| | (12281-12284), (12783-12786), (12942-12945), (13300-13303), (13323-13326), |
| | (13354-13357), (13757-13760), (13952-13955), (14024-14027), (14032-14035), |
| | (14042-14045), (14080-14083), (14150-14153), (14192-14195), (14214-14217), |
| | (14335-14338), (14758-14761), (14944-14947), (15306-15309), (15323-15326), |
| | (15332-15335), (15395-15398), (15449-15452), (16623-16626), (16703-16706), |
| | (16747-16750), (16801-16804), (16815-16818), (16919-16922), (17031-17034), |
| | (17064-17067), (17095-17098), (17101-17104), (17116-17119), (17136-17139), |
| | (17252-17255), (17268-17271), (17301-17304), (17326-17329), (17446-17449), |
| | (17523-17526), (18234-18237), (18431-18434), (18604-18607), (18783-18786), |
| | (18814-18817), (18848-18851), (18921-18924), (19031-19034), (19150-19153), |
| | (19202-19205), (19293-19296), (19492-19495), (19520-19523), (19589-19592), |
| | (19762-19765), (19929-19932), (19971-19974), (19980-19983), (20040-20043), |
| | (20076-20079), (20193-20196), (20332-20335), (20393-20396), (20408-20411), |
| | (20419-20422), (20701-20704), (20731-20734), (20788-20791), (20836-20839), |
| | (21155-21158), (21304-21307), (21394-21397), (21471-21474), (21645-21648), |
| | (21674-21677), (21990-21993), (22007-22010), (22054-22057), (22069-22072), |
| | (22087-22090), (22378-22381), (22919-22922), (23033-23036), (23065-23068), |
| | (23075-23078), (23102-23105), (23202-23205), (23228-23231), (23233-23236), |
| | (23637-23640), (23658-23661), (23668-23671), (23810-23813), (23820-23823), |
| | (23835-23838), (24018-24021), (24192-24195), (24219-24222), (24278-24281), |
| | (24300-24303), (24355-24358), (24375-24378), (24425-24428), (25100-25103), |
| | (25256-25259), (25321-25324), ((25431-25434), (25622-25625), (25660-25663), |
| | (25837-25840), (25922-25925), (26176-26179), (26263-26266), (26308-26311), |
| | (26331-26334) |
| T₄ | (638-641), (1608-1611), (2359-2362), (2378-2381), (2446-2449), (2900-2903), |
| | (3436-3439), (3446-3449), (3666-3669), (3944-3947), (3994-3997), (4109-4112), |
| | (4126-4129), (4170-4173), (4182-4185), (4637-4640), (4781-4784), (4804-4807), |
| | (5332-5335), (5343-5346), (5462-5465), (5484-5487), (5625-5628), (5639-5642), |
| | (5771-5774), (5789-5792), (6196-6199), (6224-6227), (6232-6235), (6324-6327), |
| | (6420-6423), (6614-6617), (6678-6681), (6785-6788), (7440-7443), (7555-7558), |
| | (7892-7895), (8108-8111), (8411-8414), (8452-8455), (9171-9174), (9342-9345) |
| | (9950-9953), (10134-10137), (10166-10169), (10371-10374), (10384-10387), |
| | (10391-10394), (10688-10691), (10736-10739), (10888-10891), (11160-11163), |
| | (11308-11311), (11314-11317), (11483-11486), (11530-11533), (11562-11565), |
| | (11679-11682), (11702-11705), (11716-11719), (11876-11879), (12073-12076), |
| | (12190-12193), (12363-12366), (12492-12495), (12499-12502), (12977-12980), |
| | (13140-13143), (13383-13386), (13404-13407), (13868-13871), (14220-14223), |
| | (14306-14309), (14469-14472), (14541-14544), (14697-14700), (14797-14800), |
| | (14861-14864), (14890-14893), (14993-14996), (15131-15134), (15139-15142), |
| | (15369-15372), (15388-15391), (15930-15933), (15947-15950), (16132-16135), |
| | (16526-16529), (16595-16598), (16722-16725), (16838-16841), (16895-16898), |
| | (17308-17311), (17585-17588), (18292-18295), (18481-18484), (18647-18650), |
| | (18718-18721), (19282-19285), (19576-19579), (19716-19719), (19858-19861), |
| | (19881-19884), (20021-20024), (20046-20049), (20106-20109), (20142-20145), |
| | (20961-20964), (20991-20994), (21035-21038), (21118-21121), (21546-21549), |
| | (21701-21704), (21755-21758), (21787-21790), (21817-21820), (21845-21848), |
| | (21852-21855), (21879-21882), (21904-21907), (21909-21912), (22016-22019), |
| | (22045-22048), (22235-22238) , (22963-22966), (23710-23713), (23908-23911), |
| | (23924-23927), (23965-23968), (24003-24006), (24266-24269), (24406-24409), |
| | (24604-24607), (24656-24659), (24821-24824), (25042-25045), (25070-25073), |
| | (25142-25145), (25248-25251), (25241-25244), (25397-25400), (25538-25541), |
| | (25509-25512), (26119-26122), (26219-26222) |
| G₄ | (1325-1328), (1413-1416), (2010-2013), (2018-2021), (3288-3291), (3293-3296), |
| | (8619-8622), (8837-8840), (10714-10717), (11801-11804), (11972-11975), |
| | (12034-12037), (12346-12349), (12610-12613), (13144-13147), (13497-13500), |
| | (13503-13506), (13825-13828), (14238-14241), (15226-15229), (15562-15565), |
| | (16039-16042), (16876-16879), (18375-18378), (19325-19328), (20750-20753), |
| | ((24720-24723), (25714-25717) |
| C₄ | (101-104), (229-232), (574-577), (852-855), (989-992), (1211-1214), (1255- |
| | 1258), (1342-1345), (1409-1412), (1448-1451), (1671-1674), (1826-1829), |
| | (1862-1865), (1956-1959), (2059-2062), (2107-2110), (2974-2977), (3005- |
| | 3008), (3137-3140), (4351-4354), (6467-6470), (7280-7283), (7632-7635), (8237- |
| | 8240), (8503-8506), (8912-8915), (9677-9680), (10130-10133), (10152-10155), |
| | (10582-10585), (10661-10664), (12825-12828), (13419-13422), (13431-13434), |
| | (14511-14514), (15463-15466), (15711-15714), (16087-16090), (16966-16969), |
| | (18004-18007), (18158-18161), (18253-18256), (18558-18561), (21010-21013), |
| | (21067-21070), (22498-22501), (22529-22532), (22568-22571), (22694-22697), |
| | (22952-22955) |
| A₅ | (283-287), (746-750), (1317-1321), (2685-2689), (4141-4145), (4647-4651), |
| | (4810-4814), (5392-5396), (5614-5618), (5798-5802), (5952-5956), (5961-5965), |
| | (5973-5977), (5990-5994), (6453-6457), (6503-6507), (6583-6587), 6863-6867), |
| | (7393-7397), (8626-8630), (11348-11352), (10989-10993), (12296-12300), |
| | (12928-12932), (13156-13160), (13487-13491), (16610-16614), (16930-16934), |
| | (17019-17023), (17025-17029), (17505-17509), (18662-18666), (18808-18812), |
| | (19038-19042), (19263-19267), (19778-19782), (20480-20484), (21123-21127), |
| | (21476-21480), (21620-21624), (22120-22124), (22172-22176), (22181-22185), |
| | (22276-22280), (23123-23127), (23501-23505), (23869-23873), (25126-25130), |
| | (25444-25448), (26163-26167) |
| T₅ | (2308-2312), (2316-2320), (2670-2674), (3964-3968), (4484-4488), (4584-4588), |
| | (4828-4832), (5414-5418), (5514-5518), (5821-5825), 6619-6623), 8646-8650), |
| | (8655-8659), (9230-9234), (9327-9331), (10354-10358), (12697-12701), (13594- |
| | 13598), (13678-13682), (14103-14107), (15315-15319), (15895-15899), (16517- |
| | 16521), (17240-17244), (17934-17938), (18818-18822), (21565-21569), (21574- |
| | 21578), (21797-21801), (21825-21829), (21834-21838), (24667-24671), (24804- |
| | 24808), (25411-25415), (25676-25680), (26082-26086) |
| G₅ | (6432-6436), 6728-6732), (6917-6921), (8536-8540), (10015-10019), |
| | (11828-11832), (14057-14061) |
| C₅ | (12-160), (302-306), (398-402), (1802-1806), (2123-2127), (2995-2999), (3124- |
| | 3128), 7274-7278), (14764-14768) |
| A₆ | (4629-4634), (4748-4753), (4652-4657), (5897-5902), (7233-7238), (8668-8673), |
| | (9062-9067), (11623-11628), (13260-13265), (11763-11768), (19103-19108), |
| | (19255-19260), (19600-19605), (19740-19745), (20158-20163), (21934-21939), |
| | (22284-22289), (22956-22961), (24919-24924), (25021-25026), (25224-25229) |
| T₆ | (5782-5787), (10222-10227), (10605-10610), (10938-10943), (11167-11172), |
| | (14438-14443), (17806-17811), (22816-22821), (23764-23769) |
| G₆ | (654-659), (11806-11811) |
| C₆ | (421-426), (1602-1607), (2086-2091), (10094-10099), (12880-12885), |
| | (19390-19395), (22489-22494) |
| A₇ | (2256-2262), (6060-6066), (7017-7023), (8120-8126), (8810-8816), (9700-9706), |
| | (13971-13977), (19530-19536), (20349-20355), (20057-20063), (21689-21695), |
| | (25132-25138) |
| T₇ | (2370-2376), (3936-3942), (4601-4607), 6720-6726), (9798-9804), (9864-9870), |
| | (10214-10220), (11213-11219), (16004-16010), (19011-19017), (22924-22930) |
| G₇ | (6909-6915) |
| A₈ | (2264-2271), (7934-7941), (11044-11051) |
| T₈ | (8750-8757), (9749-9756), (11721-11728) |
| T₉ | (9159-9167), (9783-9791), (14500-14508) |
| (TA)₄ | (5506-5513) |
| (GA)₄ | (9387-9394) |
| (AT)₄ | (11628-11635) |
| (AG)₄ | (11813-11820) |
| (TC)₄ | (18139-18146), (22552-22559) |
| (CT)₄ | (22640-22647), (22731-22738) |
| (CA)₅ | (22780-22789) |
| (GA)₅ | (11842-11851) |
| (CT)₈ | (8510-8525) |
| (TA)₈ | (3414-3429) |
| (CT)₁₂ | (22790-22813) |
| (AT)₂₀ | (3623-3662) |
| (CT)₂₅ | (10042-10092) |
| (TTC)₅ | (8735-8749) |
| (TTC)₈ | (10107-10130) |
| (AAAG)₃ | (8083-8094) |
| (GGAGAA)₃ | (9519-9536) |

The SSRs identified in the PDK_{—}30s944511 scaffold (GenBank accession number: GL747212-11556 nucleotides in length) and their respective locations in this scaffold are given in the following table:

| SSR | Positions occupées dans le scaffold PDK_30s944511 |
|---|---|
| A₄ | (127-130), (237-240), (295-298), (365-368), (487-490), (503-506), (512-515), (537- |
| | 540), (559-562), (583-586), (599-602), (648-651), (662-665), (732-735), (1510- |
| | 1513), (1551-1554), (1765-1768), (1816-1819), (1841-1844), (1886-1889), (2514- |
| | 2517), (2578-2581), (3016-3019), (3444-3447), (3769-3772), (5256-5259), (5695- |
| | 5698), (5787-5790), (6264-6267), (6396-6399), (6449-6452), (6730-6733), (6877- |
| | 6880), (6887-6890), (6976-6979), (7065-7068), (7070-7073), ((7201-7204), (7213- |
| | 7216), (7362-7365), (7649-7652), (7679-7682), (7723-7726), (7732-7735), (7742- |
| | 7745), (7908-7911), (7951-7954), (7994-7997), (7983-7986), (8094-8097), (8127- |
| | 8130), (8155-8158), (8270-8273), (8391-8394), (8443-8446), (8968-8971), (9077- |
| | 9080), (9649-9652), (9677-9680), (9875-9878), (10014-10017), (10352-10355), |
| | (10598-10601), (10625-10628), (10696-10699), (11008-11011), (11031-11034), |
| | (11379-11382) |
| T₄ | (290-293), (345-348), (383-386), (498-501), (721-724), (750-753), (755-758), |
| | (1491-1494), (1536-1539), (1574-1577), (1965-1968), (2185-2188), (2564-2567), |
| | (4611-4614), (4845-4848), (4950-4953), (5139-5142), (5146-5149), (5490-5493), |
| | (5558-5561), (6022-6025), (6142-6145), (6152-6155), (6178-6181), (6213-6216), |
| | (6659-6662), (6714-6717), (6855-6858), (6883-6886), (7091-7094), (7127-7130), |
| | (7132-7135), (7675-7678), (7727-7730), (7746-7749), (7770-7773), (7878-7881), |
| | (8029-8032), (8079-8082), (8089-8092), (8496-8499), (9013-9016), (9063-9066), |
| | (9338-9341), (9803-9806), (10142-10145), (10563-10566), (10781-10784), (11209- |
| | 11212), (10629-10632) |
| G₄ | (411-414), (1015-1018), (1415-1418), (3425-3428), (3464-3467), (5521-5524), |
| | (9862-9865), (11139-11142) |
| C₄ | (728-731), (770-773), (2179-2182), (2568-2571), (2766-2769), (2905-2908), (3021- |
| | 3024), (3212-3215), (3541-3544), (3966-3969), (4144-4147), (4835-4838), (6048- |
| | 6051), (6351-6354), (6486-6489), (6721-6724), (7975-7978), (9596-9599), (9736- |
| | 9739), (10479-10482), (11329-11332) |
| A₅ | (90-94), (109-113), (146-150), (355-359), (441-445), (466-470), (564-568), (604- |
| | 608), (653-657), (5764-5768), (6384-6388), (6403-6407), (7098-7102), (7151- |
| | 7155), (7327-7331), (7386-7390), (7642-7646), (7713-7717), (7752-7756), (7940- |
| | 7944), (8014-8018), (8074-8078), (8247-8251), (9795-9799), (10196-10200), |
| | (10603-10607), (10585-10589) |
| T₅ | (471-475), (701-705), (1477-1481), (4401-4405), (5155-5159), (5224-5228), (5298- |
| | 5302), (5547-5551), (6067-6071), (6160-6164), (6492-6496), (6999-7003), (7630- |
| | 7634), (7930-7934), (8259-8263), (8501-8505), (8992-8996), (9125-9129), (9809- |
| | 9813), (9890-9894), (10064-10068), (10345-10349), (11454-11458), (10715- |
| | 10719), (10670-10674) |
| G₅ | (858-862), (6752-6756), (9030-9034) |
| C₅ | (805-809), (1860-1864), (1942-1946), (2630-2634), (2802-2806), (4057-4061), |
| | (5827-5831) |
| A₆ | 6896-6901), (6867-6872), (7613-7618), (7622-7627), (7667-7672), (7694-7699), |
| | (7761-7766), (7883-7888), (8034-8039), (8046-8051), (8102-8107), (10576-10581) |
| T₆ | (283-288), (5123-5128), (7829-7834) |
| G₆ | (3430-3435) |
| C₆ | (10046-10051) |
| A₇ | (6585-6591), (10180-10186), (10374-10380) |
| T₇ | (275-281), (630-636), (10546-10552), (10800-10806), (10849-10855) |
| A₈ | (7703-7710), (7868-7875) |
| A₉ | (374-382) |
| T₉ | (711-719) |
| (CT)₄ | (2991-2998), (10129-10136), (11314-11321) |
| (AC)₄ | (6388-6395) |
| (AG)₄ | (10835-10842) |
| (TC)₅ | (11289-11298) |
| (TG)₁₀ | (11528-11547) |
| (TTGC)₃ | (4285-4296) |
| (TTAT)₄ | (9110-9125) |

The SSRs identified in the PDK_{—}30s1038101 scaffold (GenBank accession number: GL742134-34146 nucleotides in length) and their respective locations in this scaffold are given in the following table:

| SSR | Positions occupées dans le scaffold PDK_30s1038101 |
|---|---|
| A₄ | (80-83), (85-88), (483-486), (589-592), (619-622), (641-644), (820-823), (901-904), |
| | (1165-1168), (1206-1209), (1217-1220), (1391-1394), (1578-1581), (1723-1726), |
| | (1756-1759), (1787-1790), (2012-2015), (2351-2354), (2997-3000), (3133-3136), |
| | (4650-4653), (4816-4819), (4889-4892), (5097-5100), (6119-6122), (6568-6571), |
| | (6715-6719), (6806-6810), (6995-6998), (7762-7765), (7872-7875), (7657-7660), |
| | (7960-7963), (8033-8036), (8294-8297), (8899-8902), (9081-9084), (9176-9179), |
| | (9214-9217), (9336-9340), (9438-9441), (9749-9754), (9934-9937), (10209- |
| | 10213), (10275-10278), (10471-10474), (10788-10791), (10864-10868), (11118- |
| | 11122), (11132-11135), (11166-11169), (11716-11719), (12131-12134), (12176- |
| | 12179), (12481-12484), (12539-12542), (12619-12622), (12790-12793), (13275- |
| | 13278), (13410-13413), (13437-13440), (13524-13527), (13740-13743), (13905- |
| | 13908), (13926-13929), (13964-13967), (14007-14010), (14128-14131), (14140- |
| | 14143), (14163-14166), (14185-14188), (14208-14211), (14403-14406), (14490- |
| | 14493), (14524-14527), (14690-14693), (14850-14853), (14981-14984), (15107- |
| | 15110), (15161-15164), (15391-15394), (15436-15439), (15525-15528), (15568- |
| | 15571), (15576-15579), (15797-15800), (15989-15992), (16058-16061), (16155- |
| | 16158), (16225-16228), (16240-16243), (16251-16254), (16267-16270), (16336- |
| | 16339), (17044-17047), (17115-17118), (17259-17262), (17659-17662), (17693- |
| | 17696), (17985-17988), (18013-18016), (18523-18526), (19512-19515), (19665- |
| | 19668), (19708-19711), (19822-19825), (19881-19884), (19896-19899), (20154- |
| | 20157), (20159-20162), (20273-20276), (20303-20306), (20534-20537), (20664- |
| | 20667), (20759-20762), (20830-20833), (20840-20843), (20879-20882), (21092- |
| | 21095), (21249-21252), (21268-21271), (21625-21628), (21706-21709), (21755- |
| | 21758), (22004-22007), (22095-22098), (22518-22521), (22604-22607), (22805- |
| | 22808), (22812-22815), (22820-22823), (22864-22867), (22975-22978), (23046- |
| | 23049), (23149-23152), (23212-23215), (23418-23421), (23757-23760), (23843- |
| | 23846), (23858-23861), (23863-23866), (24068-24071), (24519-24522), (24658- |
| | 24661), (24711-24714), (24933-24936), (25077-25080), (25299-25302), (25838- |
| | 25841), (26352-26355), (26487-26490), (26553-26556), (26679-26682), (26721- |
| | 26724), (26780-26783), (26793-26796), (26989-26992), (27035-27038), (27523- |
| | 27526), (27583-27586), (27622-27625), (27640-27643), (27720-27723), (28078- |
| | 28081), (28099-28102), (28221-28224), (28307-28310), (28423-28426), (28559- |
| | 28562), (28650-28653), (28901-28904), (29040-29043), (29051-29054), (29273- |
| | 29276), (29441-29444), (29815-29818), (29934-29937), (29979-29982), (30027- |
| | 30030), (30111-30114), (30205-30208), (30389-30392), (30401-30404), (30532- |
| | 30535), (31011-31014), (31219-31222), (31564-31567), (32022-32025), (32330- |
| | 32333), (32349-32352), (32516-32519), (32529-32532), (32534-32537), (32794- |
| | 32797), (33116-33119), (33122-33125), (33152-33155), (33157-33160), (33304- |
| | 33307), (33499-33502), (34045-34048) |
| T₄ | (196-199), (295-298), (343-346), (380-383), (510-513), (601-604), (755-758), (917- |
| | 920 ), (930-933), (1001-1004), (1016-1019), (1058-1061), (1172-1175), (1911- |
| | 1914), (2027-2030), (2085-2088), (2233-2236), (2298-2301), (2417-2420), (2597- |
| | 2600), (2603-2606), (2922-2925), (3065-3068), (3201-3204), (3362-3365), (3367- |
| | 3370), (4702-4706), (4811-4814), (5207-5210), (5341-5344), (5503-5506), (5562- |
| | 5565), (5863-5866), (6670-6673), (6687-6690), (7137-7140), (7210-7213), (7268- |
| | 7271), (7328-7331), (7349-7352), (7518-7522), (7524-7527), (7756-7760), (8071- |
| | 8074), (9674-9678), (9941-9946), (10089-10092), (10443-10446), (10569-10573), |
| | (10598-10602), (10823-10826), (11544-11547), (11637-11640), (11649-11652), |
| | (12064-12067), (12180-12183), (12317-12320), (12420-12423), (12888-12891), |
| | (12951-12954), (12957-12960), (12980-12983), (13073-13076), (13079-13082), |
| | (13203-13206), (13249-13252), (13381-13384), (13447-13450), (14019-14022), |
| | (14107-14110), (14611-14614), (14845-14848), (14854-14857), (15826-15829), |
| | (15902-15905), (16048-16051), (16180-16183), (16185-16188), (16459-16462), |
| | (16590-16593), (16887-16890), (17226-17229), (17677-17680), (17708-17711), |
| | (17740-17743), (17949-17952), (18542-18545), (18603-18606), (18628-18631), |
| | (18651-18654), (18807-18810), (19263-19266), (19573-19576), (19733-19736), |
| | (19797-19800), (19998-20001), (20292-20295), (20596-20599), (20660-20663), |
| | (20697-20700), (20755-20758), (21385-21388), (21399-21402), (21527-21530), |
| | (21547-21550), (21621-21624), (22232-22235), (22246-22249), (22885-22888), |
| | (23107-23110), (23124-23127), (23324-23327), (23400-23403), (23584-23587), |
| | (23638-23641), (23941-23944), (23897-23900), (23908-23911), (23992-23995), |
| | (24015-24018), (24054-24057), (24193-24196), (24200-24203), (24343-24346), |
| | (25111-25114), (25682-25685), (25763-25766), (25904-25907), (25916-25919), |
| | (26342-26345), (26348-26351), (26382-26385), (27217-27220), (28275-28278), |
| | (28300-28303), (28910-28913), (28967-28970), (28994-28997), (29594-29597), |
| | (29623-29626), (29707-29710), (29773-29776), (29794-29797), (30020-30023), |
| | (30309-30312), (30727-30730), (30749-30752), (30874-30877), (31026-31029), |
| | (31052-31055), (31092-31095), (31118-31121), (31226-31229), (31253-31256), |
| | (31334-31337), (31710-31713), (31885-31888), (32010-32013), (33021-33024), |
| | (33068-33071), (33148-33151), (33163-33166), (33340-33343), (33433-33436), |
| | (33484-33487), (33575-33578), (33712-33715) |
| G₄ | (5311-5314), (5379-5382), (5477-5480), (5530-5533), (6495-6498), (7978-7981), |
| | (8152-8155), (8339-8343), (8479-8482), (10884-10887), (11711-11714), (12325- |
| | 12328), (15277-15280), (15961-15964), (16017-16020), (16839-16842), (16987- |
| | 16990), (17025-17028), (17560-17563), (17744-17747), (18142-18145), (18197- |
| | 18200), (18253-18256), (18304-18307), (19167-19170), (22028-22031), (22430- |
| | 22433), (22460-22463), (29504-29507), (31183-31186), (32158-32161), (32612- |
| | 32615), (32624-32627), (32641-32644), (32864-32867), (32929-32932), (32943- |
| | 32946), (33058-33061), (34124-34127) |
| C₄ | (171-174), (1148-1151), (2075-2078), (2145-2148), (4724-4727), (5759-5763), |
| | (5975-5978), (6043-6046), (6098-6101), (6267-6271), (6402-6405), (7036-7039), |
| | (7336-7340), (8621-8626), (980-983), (10150-10153), (12805-12808), (12859- |
| | 12862), (12871-12874), (13269-13272), (15366-15369), (15846-15849), (17216- |
| | 17219), (17364-17367), (17632-17635), (18159-18162), (18214-18217), (19549- |
| | 19552), (22788-22791), (24802-24805), (24969-24972), (25168-25171), (25532- |
| | 25535), (26934-26937), (28569-28572), (29074-29077), (30833-30836), (32453- |
| | 32456), (32752-32755), (32837-32840), (34141-34144) |
| A₅ | (28-32), (630-634), (885-889), (951-955), (1084-1088), (1152-1156), (2093-2097), |
| | (2407-2411), (3044-3048), (5966-5969), (6475-6478), (7575-7578), (8573-8578), |
| | (9593-9597), (9743-9747), (9831-9835), (9896-9899), (10424-10427), (10655- |
| | 10659), (10714-10717), (10896-10901), (11166-11169), (11380-11384), (11386- |
| | 11390), (11452-11456), (11489-11493), (13369-13373), (12845-12849), (14082- |
| | 14086), (14093-14097), (14568-14572), (14894-14898), (15410-15414), (16234- |
| | 16238), (18288-18292), (18398-18402), (18817-18821), (19603-19607), (19646- |
| | 19650), (19867-19871), (20606-20610), (20799-20803), (21665-21669), (21684- |
| | 21688), (21716-21720), (21725-21729), (22076-22080), (22239-22243), (22506- |
| | 22510), (24218-24222), (25509-25513), (25815-25819), (25846-25850), (26006- |
| | 26010), (26921-26925), (27944-27948), (28372-28376), (28405-28409), (28655- |
| | 28659), (29898-29902), (29988-29992), (30179-30183), (30230-30234), (30270- |
| | 30274), (30426-30430), (30643-30647), (32258-32262), (32270-32274), (32431- |
| | 32435), (33211-33215), (33440-33444), (33703-33707) |
| T₅ | (404-408), (736-740), (1028-1032), (3223-3227), (4749-4752), (6917-6920), (6988- |
| | 6992), (7001-7004), (7426-7429), (7820-7823), (8964-8968), (9155-9158), (9690- |
| | 9693), (10746-10749), (13650-13654), (16162-16166), (17233-17237), (17499- |
| | 17503), (17786-17790), (18707-18711), (18745-18749), (18848-18852), (19591- |
| | 19595), (19612-19616), (19876-19880), (19904-19908), (20006-20010), (21535- |
| | 21539), (23959-23963), (24428-24432), (26750-26754), (28007-28011), (28135- |
| | 28139), (28828-28832), (28999-29003), (29087-29091), (29531-29535), (29539- |
| | 29543), (29562-29566), (29739-29743), (29715-29719), (29673-29677), (29679- |
| | 29683), (30053-30057), (30149-30153), (30352-30356), (31073-31077), (31161- |
| | 31165), (31201-31205) |
| G₅ | (26162-26166), (32545-32549), (32617-32621) |
| C₅ | (25539-25543) |
| A₆ | (8651-8654), (9861-9864), (11132-11135), (14215-14220), (14319-14324), (14578- |
| | 14583), (14662-14667), (16584-16589), (17019-17024), (19622-19627), (19639- |
| | 19644), (19915-19920), (20890-20895), (21674-21679), (25945-25950), (25973- |
| | 25978), (26896-26901), (27694-27699), (28643-28648), (30418-30423), (33052- |
| | 33057), (33139-33144) |
| T₆ | (893-898), (8668-8672), (10137-10140), (12306-12311), (12853-12858), (12899- |
| | 12904), (15451-15456), (16791-16796), (19960-19965), (26599-26604), (26997- |
| | 27002), (29569-29574), (30009-30014), (31145-31150), (33426-33431) |
| G₆ | (26168-26173) |
| C₆ | (24992-24997) |
| A₇ | (12434-12440), (12463-12469), (19561-19567), (19655-19661), (19971-19977), |
| | (21473-21479), (21767-21773), (26055-26061), (29845-29851), (29924-29930), |
| | (31976-31982), (32297-32303), (33736-33742), (33736-33742) |
| T₇ | (354-360), (16448-16454), (17697-17703) |
| G₇ | (28479-28485) |
| A₈ | (15056-15063), (16063-16070), (20849-20856), (19774-19781) |
| T₈ | (29726-29733) |
| A₉ | (19629-19637) |
| A₁₀ | (12080-12089) |
| T₁₀ | (16798-16807) |
| A₁₁ | (17003-17013), (30406-30416) |
| A₁₂ | (20061-20072) |
| A₁₃ | (18033-18045) |
| A₁₅ | (33629-33643) |
| A₁₉ | (31926-31944) |
| (CT)₄ | (8629-8636) |
| (TC)₄ | (17095-17102), (17165-17172) |
| (TG)₄ | (17450-17457) |
| (TA)₄ | (18062-18069) |
| (AT)₄ | (19581-19588), (33721-33728) |
| (TC)₅ | (1884-1893) |
| (AT)₅ | (22919-22928) |
| (TC)₅ | (32824-32833) |
| (TG)₅ | (28461-28470) |
| (AT)₆ | (23302-23313), (33125-33136) |
| (TA)₇ | (427-440) |
| (AC)₁₀ | (22897-22916) |
| (TA)₁₁ | (32129-32150) |
| (ATA)₄ | (1195-1206) |
| (AAG)₇ | (26138-26158) |
| (AAAG)₃ | (17260-17271) |
| (CTAT)₃ | (17418-17429) |
| (CTTT)₃ | (29659-29670), (30338-30349) |
| (CGTAC)₃ | (16518-16532) |

The SSRs identified in the PDK_{—}30s1038231 scaffold (GenBank accession number: GL743804-23119 nucleotides in length) and their respective locations in this scaffold are given in the following table:

| SSR | Positions occupées dans le scaffold PDK_30s1038231 |
|---|---|
| A₄ | (147-150), (176-179), (216-219), (448-451), (872-875), (974-977), (1152-1155), |
| | (1182-1185), (1290-1293), (1471-1474), (1816-1819), (2050-2053), (2215-2218), |
| | (2773-2776), (2785-2788), (3261-3264), (3476-3479), (3635-3638), (3786-3789), |
| | (3815-3818), (4068-4071), (4469-4472), (4583-4586), (4888-4891), (5205-5208), |
| | (5297-5300), (5400-5403), (5564-5567), (5603-5606), (5778-5781), (5817-5820), |
| | (5915-5918), (5943-5946), (6036-6039), (6119-6122), (6693-6696), (7337-7340), |
| | (8118-8121), (8169-8172), (8235-8238), (8265-8268), (8282-8285), (9281-9284), |
| | (10272-10275), (10883-10886), (10927-10930), (11147-11150), (11176-11179), |
| | (11266-11269), (11408-11411), (11420-11423), (11428-11431), (11479-11482), |
| | (11537-11540), (11572-11575), (11957-11960), (11702-11705), (12061-12064), |
| | (12332-12335), (12832-12835), (12857-12860), (12888-12891), (13073-13076), |
| | (13162-13165), (13432-13435), (13574-13577), (13671-13674), (13710-13713), |
| | (13805-13808), (13867-13870), (13878-13881), (14658-14661), (14895-14898), |
| | (14913-14916), (15146-15149), (15440-15443), (15665-15668), (15730-15733), |
| | (15825-15828), (15859-15862), (15910-15913), (15984-15987), (16107-16110), |
| | (16166-16169), (16179-16182), (16200-16203), (16219-16222), (16535-16538), |
| | (16956-16959), (17001-17004), (17628-17631), (17666-17669), (17710-17713), |
| | (17922-17925), (17927-17930), (18090-18093), (18403-18406), (18457-18460), |
| | (18734-18737), (18746-18749), (18846-18849), (18920-18923), (18934-18937), |
| | (18939-18942), (19202-19205), (19296-19299), (19517-19520), (19587-19590), |
| | (20206-20209), (20287-20290), (20371-20374), (20495-20498), (20542-20545), |
| | (20567-20570), (20621-20624), (20663-20666), (20756-20759), (21095-21098), |
| | (21449-21452), (21810-21813), (21911-21914), (22076-22079), (22354-22357), |
| | (22370-22373), (22402-22405), (22435-22438), (22549-22552), (22608-22611), |
| | (22647-22650), (22698-22701), (23008-23011), (23019-23022) |
| T₄ | (130-133), (155-158), (246-249), (258-261), (405-408), (509-512), (902-905), |
| | (1248-1251), (1299-1302), (1407-1410), (1493-1496), (1750-1753), (2233-2236), |
| | (2282-2285), (2448-2451), (2563-2566), (2723-2726), (3307-3310), (3365-3368), |
| | (3402-3405), (3906-3909), (3950-3953), (3999-4002), (4055-4058), (4171-4174), |
| | (4241-4244), (4486-4489), (4674-4677), (4949-4952), (4982-4985), (5499-5502), |
| | (5713-5716), (6074-6077), (6086-6089), (6595-6598), (6659-6662), (7064-7067), |
| | (7571-7574), (7815-7818), (8212-8215), (8730-8733), (8886-8889), (8898-8901), |
| | (9439-9442), (10023-10026), (10224-10227), (10230-10233), (10395-10398), |
| | (10429-10432), (10463-10466), (11375-11378), (11655-11658), (11822-11825), |
| | (11867-11870), (12183-12186), (12680-12683), (12993-12996), (13000-13003), |
| | (13081-13084), (13198-13201), (13242-13245), (13285-13288), (13358-13361), |
| | (13548-13551), (13607-13610), (13645-13648), (13990-13993), (15214-15217), |
| | (15734-15737), (15807-15810), (16283-16286), (16314-16317), (16443-16446), |
| | (16541-16544), (16643-16646), (17294-17297), (17363-17366), (17583-17586), |
| | (17701-17704), (17706-17709), (17940-17943), (17959-17962), (17967-17970), |
| | (18106-18109), (18432-18435), (18657-18660), (18786-18789), (18797-18800), |
| | (18887-18890), (19237-19240), (19328-19331), (19542-19545), (19485-19488), |
| | (19499-19502), (19974-19977), (20614-20617), (20681-20684), (20891-20894), |
| | (21377-21380), (21438-21441), (21498-21501), (21878-21881), (21959-21962), |
| | (22125-22128), (22855-22858), (22907-22910), (23035-23038) |
| G₄ | (1768-1771), (2752-2755), (4565-4568), (5995-5998), (7615-7618), (8672-8675), |
| | (8691-8694), (9323-9326), (10148-10151), (12086-12089), (17390-17393), (19364- |
| | 19367), (21552-21555), (22584-22587) |
| C₄ | (473-476), (2121-2124), (2548-2551), (8529-8532), (8871-8874), (9143-9146), |
| | (10341-10344), (10351-10354), (12492-12495), (14113-14116), (15385-15388), |
| | (16004-16007), (21047-21050), (21422-21425) |
| A₅ | (27-31), (46-50), (160-164), (346-350), (409-413), (559-563), (565-569), (1117- |
| | 1121), (1340-1344), (1360-1364), (1624-1628), (2086-2090), (2733-2737), (3520- |
| | 3524), (3758-3762), (4012-4016), (4122-4126), (5179-5183), (5194-5198), (5430- |
| | 5434), (7005-7009), (7782-7786), (5644-5648), (5884-5888), (10040-10044), |
| | (10414-10418), (11840-11844), (12270-12274), (12851-12855), (13189-13193), |
| | (14589-14593), (14861-14865), (15254-15258), (15380-15384), (15868-15872), |
| | (16022-16026), (16137-16141), (16340-16344), (16528-16532), (17053-17057), |
| | (17215-17219), (18963-18967), (19053-19057), (19062-19066), (19287-19291), |
| | (19643-19647), (20685-20689), (21111-21115), (21785-21789), (21885-21889), |
| | (22004-22008), (22252-22256) |
| T₅ | (355-359), (611-615), (1142-1146), (1983-1987), (3223-3227), (3342-3346), (3506- |
| | 3510), (5173-5177), (5456-5460), (5670-5674), (6854-6858), (7466-7470), (7589- |
| | 7593), (7871-7875), (9986-9990), (10913-10917), (10970-10974), (11002-11006), |
| | (11217-11221), (11400-11404), (11488-11492), (11830-11834), (12156-12160), |
| | (12770-12774), (12784-12788), (13271-13275), (13553-13557), (13775-13779), |
| | (14634-14638), (17714-17718), (18441-18445), (18468-18472), (19799-19803), |
| | (22674-22678), (22860-22864), (22866-22870), (22947-22951) |
| G₅ | (3525-3529), (3562-3566), (4017-4021), (3863-3867), (8323-8327), (9364-9368), |
| | (9524-9528), (12112-12116), (14160-14164), (14169-14173), (14334-14338), |
| | (14406-14410), (18167-18171), (21287-21291), (23100-23104) |
| C₅ | (8905-8909), (10908-10912), (12515-12519), (21517-21521), (21523-21527) |
| A₆ | (4153-4158), (6158-6163), (11649-11654), (11882-11887), (12041-12046), (12285- |
| | 12290), (12716-12721), (12793-12798), (13169-13174), (13301-13306), (14768- |
| | 14773), (15955-15960), (16908-16913), (17887-17892), (19871-19876), (20745- |
| | 20750) |
| T₆ | (4413-4418), (4600-4605), (5286-5291), (7279-7284), (7888-7893), (8557-8562), |
| | (11944-11949), (11961-11966), (12867-12872), (15165-15170), (15641-15646), |
| | (19352-19357), (19616-19621), (21426-21431), (21443-21448), (22872-22877) |
| C₆ | (12463-12468), (18637-18642) |
| A₇ | (729-735), (5846-5852), (11080-11086), (11385-11391), (15325-15331), (15344- |
| | 15350), (20226-20232), (20391-20397), (21855-21861), (22058-22064) |
| T₇ | (1784-1790), (22778-22784) |
| A₈ | (11307-11314), (11316-11323), (11558-11565), (13053-13060) |
| T₈ | (11203-11210), (12953-12960) |
| G₈ | (10204-10211) |
| A₉ | (1088-11096), (5267-5275) |
| T₉ | (12241-12249), (12027-12035) |
| A₁₀ | (18496-18505) |
| T₁₀ | (19341-19350), (11582-11591) |
| C₁₀ | (6193-6202) |
| A₁₁ | (1954-1964) |
| (CT)₄ | (5157-5164), (21384-21391) |
| (GA)₄ | (8297-8304), (21347-21354) |
| (TA)₄ | (11189-11196), (13092-13099), (18060-18067), (22969-22976) |
| (TC)₄ | (11662-11669) |
| (AT)₄ | (12642-12649) |
| (GA)₅ | (17796-17805) |
| (CA)₅ | (20022-20031) |
| (CCTC)₄ | (21472-21487) |
| (TATG)₄ | (22975-22990) |
| (TCCAC)₃ | (17343-17357) |

The SSRs identified in the PDK_{—}30s1106761 scaffold (GenBank accession number: GL744424-20200 nucleotides in length) and their respective locations in this scaffold are given in the following table:

| SSR | Positions occupées dans le scaffold PDK_30s1106761 |
|---|---|
| A₄ | (149-152), (569-572), (792-795), (836-839), (894-897), (923-926), (1426-1429), |
| | (1600-1603), (1605-1608), (1647-1650), (1679-1682), (1688-1691), (1705-1708), |
| | (3003-3006), (3212-3215), (3223-3226), (3346-3349), (4202-4205), (4569-4572), |
| | (4596-4599), (4629-4632), (5508-5511), (5534-5537), (5929-5932), (7352-7355), |
| | (7848-7851), (8457-8460), (8473-8476), (8500-8503), (9429-9432), (9465-9468), |
| | (9732-9735), (10110-10113), (10123-10126), (10236-10239), (10445-10448), |
| | (10469-10472), (10551-10554), (10716-10719), (10781-10784), (10805-10808), |
| | (10835-10838), (10877-10880), (11423-11426), (11588-11591), (11852-11855), |
| | (12632-12635), (13268-13271), (13344-13347), (13409-13412), (13462-13465), |
| | (13622-13625), (13795-13798), (13810-13813), (13834-13837), (13846-13849), |
| | (14023-14026), (14140-14143), (14738-14741), (14952-14955), (14993-14996), |
| | (15009-15012), (15014-15017), (15394-15397), (15677-15680), (15703-15706), |
| | (16014-16017), (16120-16123), (16150-16153), (16356-16359), (16622-16625), |
| | (16714-16717), (16795-16798), (16802-16805), (16818-16821), (16827-16830), |
| | (17372-17375), (17377-17380), (17393-17396), (17402-17405), (17884-17887), |
| | (17956-17959), (18371-18374), (18387-18390), (18396-18399), (18410-18413), |
| | (19161-19164), (19321-19324), (20151-20154) |
| T₄ | (126-129), (144-147), (1412-1415), (1781-1784), (2105-2108), (2366-2369), (2469- |
| | 2472), (2505-2508), (2546-2549), (3160-3163), (4965-4968), (6231-6234), (7022- |
| | 7025), (7132-7135), (7317-7320), (7506-7509), (7561-7564), (7957-7960), (8022- |
| | 8025), (8342-8345), (8360-8363), (8408-8411), (8496-8499), (8671-8674), (8817- |
| | 8820), (8826-8829), (9006-9009), (9097-9100), (9212-9215), (9220-9223), (9238- |
| | 9241), (9273-9276), (9388-9391), (9394-9397), (9419-9422), (9667-9670), (9744- |
| | 9747), (9953-9956), (9960-9963), (10009-10012), (10280-10283), (10927-10930), |
| | (10998-11001), (11007-11010), (11265-11268), (11274-11277), (11331-11334), |
| | (11341-11344), (11383-11386), (11486-11489), (11576-11579), (11723-11726), |
| | (11729-11732), (11769-11772), (11951-11954), (12384-12387), (12430-12433), |
| | (12500-12503), (12639-12642), (12647-12650), (12661-12664), (12743-12746), |
| | (13339-13342), (13372-13375), (13420-13423), (13491-13494), (13526-13529), |
| | (13641-13644), (13686-13689), (13780-13783), (13790-13793), (13957-13960), |
| | (14131-14134), (14356-14359), (14532-14535), (14610-14613), (14646-14649), |
| | (14680-14683), (14692-14695), (14785-14788), (15248-15251), (15321-15324), |
| | (15404-15407), (15552-15555), (16111-16114), (16178-16181), (16212-16215), |
| | (16331-16334), (16618-16621), (16703-16706), (17427-17430), (17610-17613), |
| | (17696-17699), (18422-18425), (18605-18608), (18677-18680), (18803-18806), |
| | (19204-19207), (19428-19431), (20034-20037) |
| G₄ | (263-266), (526-529), (556-559), (562-565), (623-626), (690-693), (1047-1050), |
| | (3541-3544), (4521-4524), (4659-4662), (4956-4959), (5504-5507), (5461-5464), |
| | (5526-5529), (6398-6401), (6439-6442), (6478-6481), (6519-6522), (7016-7019), |
| | (8150-8153), (8188-8191), (8210-8213), (8845-8848), (10590-10593), (11848- |
| | 11851), (14493-14496), (15159-15162), (15748-15751), (15912-15915), (15929- |
| | 15932), (15991-15994), (16004-16007), (17724-17727) |
| C₄ | (800-803), (840-843), (997-1000), (1129-1132), (1297-1300), (1658-1661), (1968- |
| | 1971), (2080-2083), (2967-2970), (3114-3117), (3352-3355), (4213-4216), (4261- |
| | 4264), (4513-4516), (4901-4904), (5136-5139), (5921-5924), (6063-6066), (6384- |
| | 6387), (6505-6508), (6546-6549), (6587-6590), (6934-6937), (7979-7982), (8288- |
| | 8291), (8382-8385), (10097-10100), (10284-10287), (11112-11115), (11126- |
| | 11129), (11131-11134), (11138-11141), (11825-11828), (12070-12073), (12261- |
| | 12264), (14055-14058), (14313-14316), (15864-15867), (17487-17490), (17857- |
| | 17860), (18482-18485), (18877-18880) |
| A₅ | (850-854), (917-921), (1711-1715), (2099-2103), (2220-2224), (3377-3381), (3383- |
| | 3387), (4857-4861), (5239-5243), (6151-6155), (7737-7741), (8577-8581), (9310- |
| | 9314), (9423-9427), (10224-10228), (10678-10682), (11145-11149), (12723- |
| | 12727), (13717-13721), (14497-14501), (14509-14513), (14516-14520), (16078- |
| | 16082), (16125-16129), (16162-16166), (19340-19344), (20170-20174) |
| T₅ | (1462-1466), (1834-1838), (2432-2436), (2540-2544), (2648-2652), (2756-2760), |
| | (7517-7521), (7566-7570), (8348-8352), (8849-8853), (8862-8866), (9781-9785), |
| | (10292-10296), (10772-10776), (10839-10843), (10920-10924), (12160-12164), |
| | (12440-12444), (12450-12454), (12923-12927), (13140-13144), (13553-13557), |
| | (14667-14671), (14814-14818), (15026-15030), (15373-15377), (16264-16268), |
| | (16644-16648), (16670-16674), (17669-17673), (17675-17679), (18664-18668), |
| | (18670-18674), (19278-19282), (19487-19491), (19493-19497), (20108-20112) |
| G₅ | (357-361), (960-964), (4370-4374), (5001-5005), (6186-6190), (12775-12779), |
| | (17595-17599), (17662-17666), (19413-19417) |
| C₅ | (1801-1805), (5663-5667), (6037-6041), (11106-11110), (17970-17974) |
| A₆ | (3289-3294), (5424-5429), (9640-9645), (10748-10753), (13232-13237), (14892- |
| | 14897), (17895-17900) |
| T₆ | (8467-8472), (8486-8491), (8504-8509), (8586-8591), (8977-8982), (13439- |
| | 13444), (14759-14764), (15287-15292), (16676-16681), (16853-16858) |
| G₆ | (2821-2826), (4925-4930), (19479-19484) |
| C₆ | (1064-1069), (14819-14824), (16876-16881), (17450-17455), (18445-18450) |
| A₇ | (11410-11416), (16838-16844), (17413-17419) |
| T₇ | (8003-8009), (13225-13231), (13908-13914), (15669-15675), (16661-16667) |
| C₇ | (7384-7390) |
| A₈ | (11281-11288) |
| T₈ | (13650-13657), (8374-8381), (8392-8399), (9491-9498) |
| T₉ | (8918-8926), (9471-9479) |
| A₁₇ | (7401-7417) |
| (AG)₄ | (10387-10394) |
| (AT)₄ | (14673-14680), (15409-15416), (16782-16789) |
| (TA)₅ | (14883-14892) |
| (TC)₁₉ | (11493-11530) |
| (TGC)₄ | (12303-12314) |
| (AAAT)₃ | (16248-16259) |
| (CCCA)₃ | (17453-17464), (18448-18459) |

The SSRs identified in the PDK_{—}30s1118051 scaffold (GenBank accession number: GL739992-77159 nucleotides in length) and their respective locations in this scaffold are given in the following table:

| SSR | Positions occupées dans le scaffold PDK_30s1118051 |
|---|---|
| A₄ | (32-35), (59-62), (94-97), (321-324), (453-456), (486-489), (604-607), (678-681), |
| | (768-771), (780-783), (924-927), (11441147), (1351-1354), (1608-1611), (1672- |
| | 1675), (2418-2421), (2551-2554), (2807-2810), (2945-2948), (3098-3101), (3456- |
| | 3459), (3781-3784), (3874-3877), (3904-3907), (4517-4520), (4636-4639), (4641- |
| | 4644), (4956-4959), (5344-5347), (5437-5440), (5466-5469), (5772-5775), (5864- |
| | 5867), (5884-5887), (6216-6219), (6286-6289), (6587-6590), (6711-6714), (7149- |
| | 7152), (7504-7507), (7579-7582), (8008-8011), (8022-8025), (8405-8408), (8570- |
| | 8573), (9057-9060), (9385-9388), (10710-10713), (10865-10868), (11525-11528), |
| | (11852-11855), (12391-12394), (12878-12881), (14201-14204), (14215-14218), |
| | (14319-14322), (14918-14921), (14924-14927), (15233-15236), (15293-15296), |
| | (15382-15385), (15641-15644), (15747-15750), (16227-16230), (16612-16615), |
| | (16956-16959), (17057-17060), (17219-17222), (17296-17299), (17361-17364), |
| | (18432-18435), (18863-18866), (19138-19141), (19359-19362), (19594-19597), |
| | (19662-19665), (19870-19873), (20058-20061), (20226-20229), (20334-20337), |
| | (20397-20400), (20520-20523), (20560-20563), (20933-20936), (21234-21237), |
| | (21276-21279), (21284-21287), (21296-21299), (21337-21340), (21883-21886), |
| | (22368-22371), (22434-22437), (22713-22716), (22907-22910), (23023-23026), |
| | (23037-23040), (23428-23431), (23482-23485), (23529-23532), (23759-23762), |
| | (23852-23855), (24039-24042), (24066-24069), (24273-24276), (24869-24872), |
| | (25174-25177), (25005-25008), (25023-25026), (25359-25362), (25577-25580), |
| | (25627-25630), (25655-25658), (25692-25695), (25758-25761), (26362-26365), |
| | (26379-26382), (26548-26551), (26632-26635), (26873-26876), (27424-27427), |
| | (27827-27830), (27838-27841), (27861-27864), (27887-27890), (28360-28363), |
| | (28593-28596), (28764-28767), (30072-30075), (30130-30133), (30159-30162), |
| | (30182-30185), (30264-30267), (30599-30602), (30626-30629), (30873-30876), |
| | (30878-30881), (32856-32859), (32948-32951), (33013-33016), (33270-33273), |
| | (33365-33368), (35964-35967), (36053-36056), (36110-36113), (36115-36118), |
| | (36377-36380), (36612-36615), (36652-36655), (36870-36873), (36893-36896), |
| | (37308-37311), (37607-37610), (37915-37918), (38003-38006), (38011-38014), |
| | (38103-38106), (38160-38163), (38228-38231), (38516-38519), (38570-38573), |
| | (38884-38887), (38914-38917), (40609-40612), (38988-38991), (39317-39320), |
| | (39415-39418), (39514-39517), (40788-40791), (41219-41222), (41611-41614), |
| | (41730-41733), (41965-41968), (42002-42005), (42021-42024), (42108-42111), |
| | (42220-42223), (42272-42275), (42324-42327), (42367-42370), (42422-42425), |
| | (42709-42712), (42847-42850), (42948-42951), (42967-42970), (43572-43575), |
| | (43733-43736), (43832-43835), (44299-44302), (44905-44908), (45377-45380), |
| | (45464-45467), (45550-45553), (45755-45758), (45760-45763), (45821-45824), |
| | (46042-46045), (46270-46273), (46278-46281), (46392-46395), (46595-46598), |
| | (47181-47184), (47447-47450), (47560-47563), (47584-47587), (47645-47648), |
| | (47715-47718), (47760-47763), (48193-48196), (48244-48247), (48301-48304), |
| | (48443-48446), (48545-48548), (48555-48558), (48745-48748), (48798-48801), |
| | (48863-48866), (48884-48887), (48940-48943), (48988-48991), (49090-49093), |
| | (49292-49295), (49376-49379), (49456-49459), (49537-49540), (49547-49550), |
| | (49604-49607), (49996-49999), (50706-50709), (50741-50744), (51445-51448), |
| | (51454-51457), (51722-51725), (51730-51733), (51758-51761), (51869-51872), |
| | (52161-52164), (52456-52459), (52802-52805), (52854-52857), (53663-53666), |
| | (54088-54091), (54887-54890), (55003-55006), (55301-55304), (55439-55442), |
| | (55587-55590), (56006-56009), (56059-56062), (56155-56158), (56696-56699), |
| | (56996-56999), (57135-57138), (57210-57213), (57955-57958), (57960-57963), |
| | (57972-57975), (58100-58103), (58322-58325), (58628-58631), (58734-58737), |
| | (58922-58925), (59356-59359), (59529-59532), (60107-60110), (60355-60358), |
| | (60360-60363), (60462-60465), (60474-60477), (60585-60588), (60623-60626), |
| | (60631-60634), (60887-60890), (60980-60983), (60996-60999), (61004-61007), |
| | (61053-61056), (61237-61240), (61265-61268), (61281-61284), (62086-62089), |
| | (62325-62328), (62360-62363), (62369-62372), (62766-62769), (62923-62926), |
| | (63140-63143), (63471-63474), (63606-63609), (63619-63622), (63633-63636), |
| | (63672-63675), (63785-63788), (64421-64424), (64569-64572), (65280-65283), |
| | (65522-65525), (65575-65578), (65640-65643), (65662-65665), (65773-65776), |
| | (65833-65836), (65885-65888), (66018-66021), (66032-66035), (66343-66346), |
| | (66434-66437), (66620-66623), (66728-66731), (66747-66750), (66824-66827), |
| | (66996-66999), (67053-67056), (67193-67196), (67256-67259), (67458-67461), |
| | (67894-67897), (68977-68980), (69226-69229), (69304-69307), (69591-69594), |
| | (69986-69989), (70244-70247), (70476-70479), (70818-70821), (71077-71080), |
| | (72148-72151), (72362-72365), (72657-72660), (72977-72980), (73364-73367), |
| | (73507-73510), (73541-73544), (73677-73680), (73965-73968), (74723-74726), |
| | (74877-74880), (74966-74969), (74900-74903), (74978-74981), (75027-75030), |
| | (75108-75111), (75430-75433), (75435-75438) |
| T₄ | (63-66), (170-173), (344-347), (349-352), (371-374), (692-695), (710-713), (915- |
| | 918), (1292-1295), (1619-1622), (1652-1655), (1679-1682), (2010-2013), (2124- |
| | 2127), (2213-2216), (2232-2235), (2262-2265), (2650-2653), (2734-2737), (2794- |
| | 2797), (3048-3051), (3125-3128), (3387-3390), (3438-3441), (3473-3476), (3807- |
| | 3810), (3856-3859), (3863-3866), (3923-3926), (4345-4348), (4543-4546), (4548- |
| | 4551), (4600-4603), (4982-4985), (5031-5034), (6193-6196), (5178-5181), (5407- |
| | 5410), (5420-5423), (5448-5451), (5453-5456), (5494-5497), (5517-5520), (5846- |
| | 5849), (5903-5906), (5911-5914), (6243-6246), (6613-6616), (6662-6665), (6729- |
| | 6732), (6835-6838), (6960-6963), (7059-7062), (7102-7105), (7136-7139), (7153- |
| | 7156), (7168-7171), (7391-7394), (7484-7487), (7595-7598), (7876-7879), (8004- |
| | 8007), (8012-8015), (8399-8402), (8412-8415), (8425-8428), (8726-8729), (8892- |
| | 8895), (8904-8907), (9028-9031), (9034-9038), (9044-9047), (9068-9071), (9258- |
| | 9261), (9314-9317), (9331-9334), (9482-9485), (9498-9501), (9602-9605), (9666- |
| | 9669), (9887-9890), (10042-10045), (10074-10077), (10356-10359), (10399- |
| | 10402), (10501-10504), (10717-10720), (10967-10970), (11007-11010), (11035- |
| | 11038), (11236-11239), (10749-10752), (11674-11677), (11716-11719), (11764- |
| | 11767), (11830-11833), (11910-11913), (12007-12010), (12106-12109), (8226- |
| | 8229), (12268-12271), (12481-12484), (12584-12587), (13809-13812), (13935- |
| | 13938), (14941-14944), (20254-20257), (20275-20278), 14964-14967), (15280- |
| | 15283), (15366-15369), (15696-15699), (15757-15760), (15811-15814), (16133- |
| | 16136), (16408-16411), (16659-16662), (16723-16726), (16903-16906), (17063- |
| | 17066), (17127-17130), (17257-17260), (17270-17273), (17319-17322), (17342- |
| | 17345), (17385-17388), (17456-17459), (17507-17510), (17512-17515), (17540- |
| | 17543), (17653-17656), (17707-17710), (17799-17802), (17991-17994), (18035- |
| | 18038), (18229-18232), (18525-18528), (19021-19024), (18878-18881), (19411- |
| | 19414), (19439-19442), (19738-19741), (19911-19914), (20039-20042), (20149- |
| | 20152), (20156-20159), (20536-20539), (20581-20584), (20820-20823), (20861- |
| | 20864), (20894-20897), (21186-21189), (22085-22088), (22294-22297), (22377- |
| | 22380), (22447-22450), (22554-22557), (22585-22588), (22699-22702), (22708- |
| | 22711), (22895-22898), (22934-22937), (23300-23303), (23829-23832), (23894- |
| | 23897), (23910-23913), (24296-24299), (24382-24385), (24687-24690), (24793- |
| | 24796), (24928-24931), (25062-25065), (25152-25155), (25159-25162), (25202- |
| | 25205), (25252-25255), (25354-25357), (25416-25419), (25467-25470), (25646- |
| | 25649), (25696-25699), (25707-25710), (25938-25941), (26224-26227), (26391- |
| | 26394), (26457-26460), (26468-26471), (26587-26590), (26637-26640), (26645- |
| | 26648), (26726-26729), (26850-26853), (27515-27518), (27697-27700), (27893- |
| | 27896), (27981-27984), (28005-28008), (28128-28131), (28226-28229), (28379- |
| | 28382), (28478-28481), (28483-28486), (28625-28628), (28708-28711), (28724- |
| | 28727), (29254-29257), (29864-29867), (30152-30155), (30233-30236), (30257- |
| | 30260), (30314-30317), (30454-30457), (30454-30457), (30585-30588), (30659- |
| | 30662), (30883-30886), (31083-31086), (31244-31247), (32825-32828), (32835- |
| | 32838), (33227-33230), (33388-33391), (35638-35641), (36700-36703), (37128- |
| | 37131), (37142-37145), (37211-37214), (37248-37251), (37651-37654), (37778- |
| | 37781), (37855-37858), (37902-37905), (37939-37942), (38007-38010), (38107- |
| | 38110), (38375-38378), (38474-38477), (39177-39180), (39304-39307), (40536- |
| | 40539), (40636-40639), (40685-40688), (41560-41563), (41898-41901), (42085- |
| | 42088), (42379-42382), (42384-42387), (42762-42765), (42782-42785), (43029- |
| | 43032), (43169-43172), (43347-43350), (43352-43355), (43425-43428), (43540- |
| | 43543), (43639-43642), (43675-43678), (43989-43992), (44020-44023), (44325- |
| | 44328), (44364-44367), (44735-44738), (44766-44769), (44820-44823), (44844- |
| | 44847), (45027-45030), (45107-45110), (45210-45213), (45902-45905), (45907- |
| | 45910), (45948-45951), (46274-46277), (46309-46312), (46325-46328), (46464- |
| | 46467), (46571-46574), (46619-46622), (46637-46640), (46747-46750), (46920- |
| | 46923), (47317-47320), (47322-47325), (47364-47367), (47459-47462), (47497- |
| | 47500), (47739-47742), (47851-47854), (47856-47859), (47999-48002), (48371- |
| | 48374), (48433-48436), (48689-48692), (48723-48726), (48787-48790), (48905- |
| | 48908), (48992-48995), (49096-49099), (49229-49232), (49260-49263), (49561- |
| | 49564), (49893-49896), (50088-50091), (50117-50120), (51050-51053), (51161- |
| | 51164), (51305-51308), (51449-51452), (51508-51511), (51629-51632), (51709- |
| | 51712), (52356-52359), (52552-52555), (52660-52663), (52751-52754), (52828- |
| | 52831), (53071-53074), (53490-53493), (53560-53563), (53792-53795), (53816- |
| | 53819), (53834-53837), (54041-54044), (54213-54216), (54314-54317), (54327- |
| | 54330), (54397-54400), (54603-54606), (54719-54722), (54837-54840), (54958- |
| | 54961), (55293-55296), (55320-55323), (55505-55508), (55534-55537), (56055- |
| | 56058), (56320-56323), (56352-56355), (56362-56365), (56372-56375), (56431- |
| | 56434), (56792-56795), (56806-56809), (56837-56840), (57111-57114), (57265- |
| | 57268), (57328-57331), (57517-57520), (57636-57639), (57665-57668), (57710- |
| | 57713), (57793-57796), (58146-58149), (58272-58275), (58280-58283), (58292- |
| | 58295), (58303-58306), (58330-58333), (58379-58382), (58601-58604), (58910- |
| | 58913), (59155-59158), (59205-59208), (59337-59340), (59495-59498), (59752- |
| | 59755), (59763-59766), (59789-59792), (60025-60028), (60040-60043), (60084- |
| | 60087), (60627-60630), (60917-60920), (60935-60938), (60949-60952), (60988- |
| | 60991), (61061-61064), (61089-61092), (61434-61437), (61743-61746), (61755- |
| | 61758), (62004-62007), (62163-62166), (62257-62260), (62453-62456), (62965- |
| | 62968), (62989-62992), (63385-63388), (63750-63753), (64405-64408), (64596- |
| | 64599), (64612-64615), (64634-64637), (64808-64811), (65246-65249), (65387- |
| | 65390), (65474-65477), (65513-65516), (65620-65623), (65705-65708), (65814- |
| | 65817), (65837-65840), (65891-65894), (66077-66080), (66164-66167), (66176- |
| | 66179), (66290-66293), (66307-66310), (66321-66324), (66724-66727), (67048- |
| | 67051), (67138-67141), (67405-67408), (67694-67697), (67936-67939), (68100- |
| | 68103), (68439-68442), (69252-69255), (69539-69542), (70734-70737), (72323- |
| | 72326), (72498-72501), (72710-72713), (72911-72914), (72928-72931), (72957- |
| | 72960), (73292-73295), (73415-73418), (73473-73476), (73854-73857), (74001- |
| | 74004), (74130-74133), (74167-74170), (74228-74231), (74300-74303), (74410- |
| | 74413), (74426-74429), (74470-74473), (74653-74656), (74674-74677), (74954- |
| | 74957), (75072-75075), (75412-75415), (75548-75551), (75961-75964), (75996- |
| | 75999), (76272-76275), (76290-76293), (76347-76350), (76420-76423), (76448- |
| | 76451), (76620-76623), (76892-76895), (77042-77045) |
| G₄ | (732-735), (951-954), (1206-1209), (1214-1217), (8669-8672), (8779-8782), (9724- |
| | 9727), (10129-10132), (10594-10597), (12059-12062), (16117-16120), (16302- |
| | 16305), (16311-16314), (16480-16483), (16616-16619), (16652-16655), (17199- |
| | 17202), (17204-17207), (18089-18092), (19198-19201), (23262-23265), (22523- |
| | 22526), (23638-23641), (28346-28349), (29665-29668), (30018-30021), (32717- |
| | 32720), (36329-36332), (37273-37276), (37455-37458), (37516-37519), (37729- |
| | 37732), (38144-38147), (38727-38730), (43375-43378), (43400-43403), (45978- |
| | 45981), (46444-46447), (46966-46969), (49569-49572), (50730-50733), (50921- |
| | 50924), (52037-52040), (52623-52626), (53418-53421), (53761-53764), (54096- |
| | 54099), (54224-54227), (56063-56066), (56138-56141), (56246-56249), (57951- |
| | 57954), (58019-58022), (59177-59180), (60398-60401), (61736-61739), (64857- |
| | 64860), (64903-64906), (69019-69022), (69468-69471), (71850-71853), (71879- |
| | 71882), (72091-72094), (72185-72188), (73057-73060), (75042-75045), (75820- |
| | 75823), (76112-76115), (76276-76279) |
| C₄ | (586-589), (682-685), (788-791), (942-945), (2954-2957), (3193-3196), (4717- |
| | 4720), (8351-8354), (8434-8437), (8764-8767), (8833-8836), (9174-9177), (10300- |
| | 10303), (12184-12187), (12702-12705), (14149-14152), (14829-14832), (14886- |
| | 14889), (15154-15157), (16037-16040), (17545-17548), (19180-19183), (20508- |
| | 20511), (22364-22367), (23315-23318), (26423-26426), (27008-27011), (29691- |
| | 29694), (29883-29886), (30346-30349), (35831-35834), (36160-36163), (36547- |
| | 36550), (39084-39087), (42259-42262), (42477-42480), (43370-43373), (44027- |
| | 44030), (47072-47075), (47735-47738), (47753-47756), (50851-50854), (54677- |
| | 54680), (56279-56282), (58461-58464), (62628-62631), (63972-63975), (64008- |
| | 64011), (64260-64263), (64458-64461), (65952-65955), (67490-67493), (67655- |
| | 67658), (70013-70016), (70119-70122), (70541-70544), (70958-70961), (71051- |
| | 71054), (71732-71735), (76454-76457), (76988-76991) |
| A₅ | (1365-1369), (2464-2468), (3018-3022), (3066-3070), (3361-3365), (3834-3838), |
| | (1740-1744), (1849-1853), (4610-4614), (5002-5006), (5390-5394), (6167-6171), |
| | (6261-6265), (6633-6637), (6680-6684), (7028-7032), (7073-7077), (7550-7554), |
| | (7974-7978), (7573-7577), (7608-7612), (8139-8143), (8174-8178), (9108-9112), |
| | (9148-9152), (11132-11136), (11396-11400), (11613-11617), (11904-11908), |
| | (12337-12341), (12889-12893), (14775-14779), (15085-15089), (15241-15245), |
| | (15581-15585), (16384-16388), (16547-16551), (16566-16570), (16601-16605), |
| | (17445-17449), (18093-18097), (18373-18377), (18417-18421), (19040-19044), |
| | (20311-20315), (21059-21063), (21256-21260), (22089-22093), (22611-22615), |
| | (23200-23204), (24044-24048), (26937-26941), (28061-28065), (28150-28154), |
| | (28515-28519), (31024-31028), (32847-32851), (36461-36465), (36718-36722), |
| | (37224-37228), (38321-38325), (38453-38457), (40763-40767), (42374-42378), |
| | (45747-45751), (45834-45838), (46240-46244), (47213-47217), (47388-47392), |
| | (47657-47661), (49556-49560), (51692-51696), (52221-52225), (52439-52443), |
| | (52729-52733), (52759-52763), (53929-53933), (54707-54711), (55033-55037), |
| | (56357-56361), (56972-56976), (60651-60655), (60726-60730), (61022-61026), |
| | (61045-61049), (61097-61101), (61138-61142), (61270-61274), (62907-62911), |
| | (64607-64611), (64625-64629), (64655-64659), (64662-64666), (64974-64978), |
| | (65599-65603), (65903-65907), (66834-66838), (66862-66866), (67198-67202), |
| | (67861-67865), (68613-68617), (68854-68858), (69414-69418), (70051-70055), |
| | (70797-70801), (70890-70894), (76037-76041) |
| T₅ | (142-146), (209-213), (338-342), (357-361), (798-802), (1592-1596), (2678-2682), |
| | (2960-2964), (3003-3007), (3010-3014), (3061-3065), (3074-3078), (3083-3087), |
| | (3228-3232), (3392-3396), (3517-3521), (3620-3624), (3812-3816), (3881-3885), |
| | (3890-3894), (1838-1842), (3953-3957), (4485-4489), (4577-4581), (4592-4596), |
| | (4605-4609), (4618-4622), (4627-4631), (4684-4688), (4768-4772), (4987-4991), |
| | (4994-4998), (5036-5040), (5042-5046), (5057-5061), (5096-5100), (5382-5386), |
| | (5432-5436), (5784-5788), (5802-5806), (5870-5874), (5997-6001), (6198-6202), |
| | (6205-6209), (6250-6254), (6256-6260), (6271-6275), (6618-6622), (6625-6629), |
| | (6669-6673), (6675-6679), (6688-6692), (6697-6701), (6759-6763), (6843-6847), |
| | (7065-7069), (7107-7111), (7127-7131), (7280-7284), (7489-7493), (7557-7561), |
| | (7566-7570), (8031-8035), (8154-8158), (9230-9234), (9547-9551), (9915-9919), |
| | (10158-10162), (10667-10671), (10683-10687), (11078-11082), (11427-11431), |
| | (11861-11865), (12038-12042), (12788-12792), (14208-14212), (15123-15127), |
| | (15258-15262), (16294-16298), (16870-16874), (16895-16899), (16926-16930), |
| | (17790-17794), (18140-18144), (18481-18485), (18610-18614), (18625-18629), |
| | (18704-18708), (18979-18983), (19465-19469), (20004-20008), (20881-20885), |
| | (22051-22055), (22539-22543), (22939-22943), (23052-23056), (23310-23314), |
| | (24922-24926), (25067-25071), (25732-25736), (26607-26611), (28106-28110), |
| | (28140-28144), (28583-28587), (30800-30804), (30866-30870), (30969-30973), |
| | (31018-31022), (32952-32956), (35944-35948), (35968-35972), (36046-36050), |
| | (36058-36062), (36600-36604), (36737-36741), (36767-36771), (37055-37059), |
| | (37093-37097), (37427-37431), (37720-37724), (38211-38215), (38622-38626), |
| | (38664-38668), (38681-38685), (38769-38773), (38944-38948), (38975-38979), |
| | (40781-40785), (41413-41417), (42100-42104), (42132-42136), (42402-42406), |
| | (42552-42556), (42599-42603), (42879-42883), (43147-43151), (44163-44167), |
| | (44243-44247), (44285-44289), (44412-44416), (44963-44967), (45003-45007), |
| | (45077-45081), (45083-45087), (45262-45266), (45329-45333), (45339-45343), |
| | (45365-45369), (45510-45514), (46385-46389), (46489-46493), (47617-47621), |
| | (47369-47373), (48680-48684), (48842-48846), (48911-48915), (49490-49494), |
| | (49585-49589), (49637-49641), (49648-49652), (49787-49791), (49913-49917), |
| | (50080-50084), (51203-51207), (51794-51798), (51261-51265), (51267-51271), |
| | (52076-52080), (52581-52585), (52679-52683), (53050-53054), (54939-54943), |
| | (55022-55026), (55280-55284), (55482-55486), (55881-55885), (56310-56314), |
| | (56386-56390), (58006-58010), (58503-58507), (58536-58540), (59081-59085), |
| | (59238-59242), (59876-59880), (60008-60012), (60481-60485), (60534-60538), |
| | (60676-60680), (61031-61035), (61039-61043), (61070-61074), (61125-61129), |
| | (61393-61397), (61771-61775), (62068-62072), (62940-62944), (63001-63005), |
| | (63007-63011), (63020-63024), (63581-63585), (63667-63671), (63679-63683), |
| | (63741-63745), (65149-65153), (65482-65486), (65557-65561), (67714-67718), |
| | (72467-72471), (72730-72734), (72760-72764), (74395-74399), (74443-74447), |
| | (74496-74500), (74544-74548), (74662-74666), (74815-74819), (75142-75146), |
| | (75222-75226), (76177-76181), (76210-76214), (76763-76767), (76820-76824) |
| G₅ | (8363-8367), (8486-8490), (11494-11498), (18103-18107), (21308-21312), (21818- |
| | 21822), (29224-29228), (31095-31099), (31153-31157), (37475-37479), (37657- |
| | 37661), (50901-5090550715-50719), (50723-50727), (55305-55309), (60153- |
| | 60157), (61628-61632), (61634-61638), (62667-62671), (62827-62831), (72023- |
| | 72027), (72063-72067), (75185-75189) |
| C₅ | (24401-24405), (23247-23251), (26659-26663), (49347-49351), (63525-63529), |
| | (71269-71273), (71491-71495), (75231-75235), (76011-76015), (76925-76929) |
| A₆ | (929-934), (2833-2838), (2936-2941), (3025-3030), (3131-3136), (3408-3413), |
| | (3523-3528), (3827-3832), (3897-3902), (3935-3940), (4562-4567), (4569-4574), |
| | (5076-5081), (5083-5088), (5459-5464), (5816-5821), (5823-5828), (5877-5882), |
| | (6639-6644), (6704-6709), (7119-7124), (7142-7147), (8051-8056), (8102-8107), |
| | (12782-12787), (15735-15740), (16992-16997), (19390-19395), (21170-21175), |
| | (21265-21270), (23103-23108), (27414-27419), (28416-28421), (30137-30142), |
| | (30496-30501), (30558-30563), (36472-36477), (37230-37235), (38070-38075), |
| | (41295-41300), (41552-41557), (42123-42128), (42155-42160), (46097-46102), |
| | (49363-49368), (51215-51220), (60295-60300), (62586-62591), (66023-66028), |
| | (66182-66187), (66551-66556), (67303-67308), (68750-68755), (73095-73100), |
| | (73198-73203), (75463-75468) |
| T₆ | (5375-5380), (3053-3058), (3443-3448), (3450-3455), (3501-3506), (3535-3540), |
| | (3818-3823), (3868-3873), (5425-5430), (5602-5607), (5851-5856), (5858-5863), |
| | (5930-5935), (6280-6285), (6401-6406), (6737-6742), (7176-7181), (7544-7549), |
| | (7616-7621), (8879-8884), (9786-9791), (10515-10520), (11362-11367), (12031- |
| | 12036), (10934-10939), (17193-17198), (18583-18588), (19274-19279), (19996- |
| | 20001), (20162-20167), (21514-21519), (21527-21532), (22059-22064), (24395- |
| | 24400), (25257-25262), (25851-25856), (25944-25949), (26352-26357), (26430- |
| | 26435), (27932-27937), (28072-28077), (28422-28427), (30044-30049), (36381- |
| | 36386), (36632-36637), (36670-36675), (37062-37067), (38588-38593), (39134- |
| | 39139), (42310-42315), (42333-42338), (42454-42459), (42471-42476), (44485- |
| | 44490), (44885-44890), (46341-46346), (51060-51065), (51295-51300), (53495- |
| | 53500), (53565-53570), (53886-53891), (55191-55196), (55496-55501), (55527- |
| | 55532), (55839-55844), (56293-56298), (56616-56621), (56656-56661), (57180- |
| | 57185), (57319-57324), (57400-57405), (57726-57731), (58576-58581), (59011- |
| | 59016), (60065-60070), (60550-60555), (60851-60856), (60870-60875), (62512- |
| | 62517), (63299-63304), (63724-63729), (72028-72033), (74528-74533), (74771- |
| | 74776), (74894-74899), (76241-76246), (76258-76263) |
| G₆ | (21355-21360), (43058-43063), (50769-50774), (64062-64067) |
| C₆ | 12559-12564), (15179-15184), (32871-32876), (38027-38032), (51038-51043), |
| | (64023-64028), (71637-71642), (73500-73505) |
| A₇ | (3090-3096), (3494-3500), (5499-5505), (7182-7188), (8041-8047), (8145-8151), |
| | (15462-15468), (16778-16784), (18200-18206), (30464-30470), (30789-30795), |
| | (35952-35958), (37686-37692), (47347-47353), (51593-51599), (60280-60286), |
| | (61336-61342), (63730-63736), (64617-64623), (72687-72693), (73220-73226) |
| T₇ | (248-254), (5477-5483), (7194-7200), (9958-9964), (7625-7631), (19428-19434), |
| | (21876-21882), (24774-24780), (24836-24842), (25540-25546), (30219-30225), |
| | (30397-30403), (37183-37189), (38711-38717), (38957-38963), (44992-44998), |
| | (46330-46336), (48710-48716), (49206-49212), (53017-53023), (53534-53540), |
| | (53779-53785), (57087-57093), (60056-60062), (61403-61409), (63283-63289), |
| | (64648-64654), (71131-71137), (72611-72617), (76047-76053), (76196-76202), |
| | (76798-76804) |
| C₇ | (2163-2169), (40801-40807), (56873-56879) |
| G₇ | (31087-31093) |
| A₈ | (3479-3486), (4670-4677), (5396-5403), (17516-17523), (22807-22814), (30172- |
| | 30179), (33099-33106), (36179-36186), (36348-36355), (42293-42300), (49281- |
| | 49288), (48487-48494), (53285-53292), (60956-60963), (62129-62136), (72618- |
| | 72625) |
| T₈ | (748-755), (1318-1325), (9221-9228), (19288-19295), (25184-25191), (27059- |
| | 27066), (42353-42360), (47306-47313), (49082-49089), (54443-54450), (61817- |
| | 61824), (63709-63716), (75455-75462) |
| A₉ | (6324-6332), (64558-64566), (42633-42641) |
| T₉ | (9236-9244), (20851-20859), (37802-37810), (48736-48744), (51664-51672), |
| | (52066-52074), (75299-75307) |
| G₉ | (64072-64080) |
| A₁₀ | (2535-2544), (2967-2976), (6743-6752), (42045-42054), (51913-51922), (61833- |
| | 61842) |
| T₁₀ | (9247-9256), (9320-9329), (42197-42206), (45061-45070), (54007-54016), (57383- |
| | 57392), (61011-61020), (76324-76333) |
| C₁₀ | (56862-56871) |
| A₁₁ | (72425-72435), (75492-75502) |
| T₁₁ | (43526-43536) |
| C₁₁ | (42929-42939) |
| A₁₂ | (62598-62609) |
| T₁₂ | (43588-43599), (47238-47249), (59940-59951) |
| T₁₄ | (57247-57260), (63592-63605) |
| T₁₆ | (63348-63363) |
| T₁₇ | (2063-2079) |
| A₈₄ | (56886-56969) |
| (AT)₄ | (129-136), (3528-3535), (7516-7523), (42223-42230), (42833-42840), (50236- |
| | 50243), (50486-50493), (50578-50585), (53186-53193), (66387-66394) |
| (TC)₄ | (330-337), (50983-50990) |
| (TA)₄ | (12674-12681), (23331-23338), (27911-27918), (50381-50388) |
| (TG)₄ | (13858-13865), (26333-26340), (45542-45549), (39341-39348), (50219-50226), |
| | (50447-50454) |
| (CA)₄ | (38365-38372), (65338-65345), (50744-50751), (64575-64582), |
| (GC)₄ | (61508-61515) |
| (GA)₄ | (50905-50912), (68011-68018) |
| (CT)₄ | (51043-51050), (68079-68086) |
| (GT)₄ | (65941-65948) |
| (AT)₅ | (41477-41486), (50438-50447) |
| (TG)₅ | (49759-49768) |
| (GA)₅ | (50774-50783) |
| (GT)₅ | (2081-2090) |
| (AG)₅ | (72172-72181) |
| (AT)₆ | (50330-50341) |
| (AG)₉ | (61703-61720) |
| (AT)₉ | (67326-67343) |
| (TC)₁₆ | (61475-61507) |
| (AC)₁₉ | (50256-50294) |
| (AT)₃₄ | (49692-49759) |
| (CAC)₄ | (3744-3755) |
| (CCA)₄ | (4924-4935) |
| (CAA)₄ | (7209-7220) |
| (TTA)₄ | (7283-7294) |
| (CGC)₄ | (7227172282) |
| (TAT)₅ | (7521-7535) |
| (TCT)₅ | (8705-8719) |
| (ATGT)₃ | (10083-10094) |
| (AGGG)₃ | (21155-21166) |
| (TTTC)₃ | (43676-43687), (76893-76904), |
| (GGTT)₃ | (44606-44617) |
| (TTAT)₃ | (50069-50080) |
| (AGGG)₃ | (56009-56020) |
| (CTTT)₃ | (76058-76069) |
| (AATA)₄ | (11265-11280) |
| (CTAT)₄ | (26304-26319) |
| (TTTG)₄ | (76331-76346) |
| (GTAT)₇ | (1-28) |
| (TAATA)₃ | (62009-62023) |
| (ATATA)₃ | (63801-63815) |
| (ATAAT)₃ | (64500-64514) |
| (ACCCA)₄ | (18765-18784) |
| (GCCATC)₃ | (7368-7385) |

The SSRs identified in the PDK_{—}30s1150131 scaffold (GenBank accession number: GL740885-50308 nucleotides in length) and their respective locations in this scaffold are given in the following table:

| SSR | Positions occupées dans le scaffold PDK_30s1150131 |
|---|---|
| A₄ | (41-44), (61-64), (141-144), (197-200), (264-267), (458-461), (476-479), (623-626), |
| | (840-843), (979-982), (1008-1011), (1013-1016), (1039-1042), (1070-1073), (1282- |
| | 1285), (1287-1290), (1437-1440), (1481-1484), (1586-1589), (1966-1969), (1991- |
| | 1994), (2044-2047), (2151-2154), (2385-2388), (3351-3354), (3536-3539), (3668- |
| | 3671), (3922-3925), (4056-4059), (4140-4143), (4259-4262), (4491-4494), (4634- |
| | 4637), (4651-4654), (4656-4659), (4680-4683), (4762-4765), (4809-4812), (4991- |
| | 4994), (5102-5105), (5496-5499), (5533-5536), (5564-5567), (5710-5713), (6186- |
| | 6189), (6335-6338), (6341-6344), (6469-6472), (6508-6511), (6653-6656), (6679- |
| | 6682), (6750-6753), (6766-6769), (6868-6871), (7199-7202), (7319-7322), (7336- |
| | 7339), (7651-7654), (7889-7892), (7928-7931), (7959-7962), (8121-8124), (8129- |
| | 8132), (8342-8345), (8595-8598), (8680-8683), (8764-8767), (8805-8808), (9018- |
| | 9021), (9569-9572), (9625-9628), (9894-9897), (10095-10098), (10254-10257), |
| | (10893-10896), (11259-11262), (11600-11603), (11728-11731), (11892-11895), |
| | (12131-12134), (12652-12655), (12676-12679), (12722-12725), (12896-12899), |
| | (12951-12954), (13248-13251), (13262-13265), (13338-13341), (13417-13420), |
| | (13572-13575), (13614-13617), (13635-13638), (13672-13675), (13732-13735), |
| | (13896-13899), (13959-13962), (14099-14102), (14159-14162), (14505-14508), |
| | (14527-14530), (14819-14822), (14842-14845), (15031-15034), (15374-15377), |
| | (15447-15450), (15689-15692), (15774-15777), (15984-15987), (16189-16192), |
| | (16223-16226), (16309-16312), (16334-16337), (16395-16398), (16472-16475), |
| | (16526-16529), (16541-16544), (16779-16782), (16801-16804), (16904-16907), |
| | (17000-17003), (17317-17320), (17458-17461), (17650-17653), (17777-17780), |
| | (17798-17801), (17950-17953), (17961-17964), (17966-17969), (18063-18066), |
| | (18187-18190), (18302-18305), (18359-18362), (18472-18475), (18706-18709), |
| | (18724-18727), (18734-18737), (18774-18777), (19075-19078), (19392-19395), |
| | (19398-19401), (19645-19648), (19751-19754), (20245-20248), (20255-20258), |
| | (20347-20350), (20357-20360), (20402-20405), (20418-20421), (20535-20538), |
| | (20564-20567), (20591-20594), (20606-20609), (20631-20634), (20658-20661), |
| | (20785-20788), (20850-20853), (20996-20999), (21088-21091), (21230-21233), |
| | (21754-21757), (22253-22256), (22376-22379), (22531-22534), (22584-22587), |
| | (22711-22714), (22908-22911), (22999-23002), (23129-23132), (23166-23169), |
| | (23281-23284), (23320-23323), (23432-23435), (23934-23937), (24083-24086), |
| | (24223-24226), (24236-24239), (24321-24324), (24339-24342), (24374-24377), |
| | (24578-24581), (24924-24927), (24994-24997), (24999-25002), (25893-25896), |
| | (25948-25951), (26026-26029), (26053-26056), (26066-26069), (26151-26154), |
| | (26228-26231), (26309-26312), (26596-26599), (26978-26981), (27040-27043), |
| | (27082-27085), (27109-27112), (27489-27492), (27553-27556), (27639-27642), |
| | (27658-27661), (27690-27693), (27813-27816), (27881-27884), (27913-27916), |
| | (27975-27978), (28074-28077), (28127-28130), (28597-28600), (28646-28649), |
| | (28843-28846), (28853-28856), (28943-28946), (29086-29089), (29130-29133), |
| | (29196-29199), (29224-29227), (29294-29297), (29321-29324), (30514-30517), |
| | (30603-30606), (30653-30656), (30670-30673), (30703-30706), (30745-30748), |
| | (31066-31069), (31357-31360), (31528-31531), (31911-31914), (31921-31924), |
| | (32023-32026), (32407-32410), (32591-32594), (32680-32683), (32834-32837), |
| | (32922-32925), (33136-33139), (33152-33155), (33366-33369), (33931-33934), |
| | (33938-33941), (33957-33960), (34118-34121), (34342-34345), (34560-34563), |
| | (34833-34836), (35083-35086), (35106-35109), (35720-35723), (35770-35773), |
| | (35876-35879), (35893-35896), (35953-35956), (35958-35961), (36037-36040), |
| | (36455-36458), (36571-36574), (36810-36813), (37244-37247), (37435-37438), |
| | (37692-37695), (37961-37964), (38137-38140), (38170-38173), (38321-38324), |
| | (38332-38335), (38349-38352), (38383-38386), (38409-38412), (38425-38428), |
| | (38596-38599), (38703-38706), (38898-38901), (38922-38925), (38994-38997), |
| | (39066-39069), (39288-39291), (39294-39297), (39326-39329), (39476-39479), |
| | (39760-39763), (39833-39836), (39999-40002), (40017-40020), (40236-40239), |
| | (40368-40371), (40435-40438), (40507-40510), (40540-40543), (40589-40592), |
| | (40713-40716), (40743-40746), (40751-40754), (40803-40806), (41446-41449), |
| | (41578-41581), (42049-42052), (42402-42405), (42922-42925), (43584-43587), |
| | (43608-43611), (43647-43650), (44046-44049), (44220-44223), (44228-44231), |
| | (44238-44241), (44248-44251), (44326-44329), (44362-44365), (44402-44405), |
| | (44414-44417), (44575-44578), (45218-45221), (45250-45253), (45271-45274), |
| | (45345-45348), (45537-45540), (45586-45589), (45660-45663), (45702-45705), |
| | (45707-45710), (45860-45863), (46154-46157), (46162-46165), (46368-46371), |
| | (46373-46376), (46661-46664), (46921-46924), (47105-47108), (47372-47375), |
| | (47850-47853), (47970-47973), (48160-48163), (48181-48184), (48475-48478), |
| | (49378-49381), (49470-49473), (49610-49613), (49663-49666), (49899-49902), |
| | (50049-50052), (50079-50082), (50097-50100), (50102-50105), (50171-50174), |
| | (50201-50204), (50283-50286) |
| T₄ | (259-262), (521-524), (848-851), (862-865), (1019-1022), (1052-1055), (1312- |
| | 1315), (1491-1494), (1611-1614), (1689-1692), (1725-1728), (1735-1738), (1882- |
| | 1885), (1946-1949), (2008-2011), (2090-2093), (2230-2233), (2465-2468), (2598- |
| | 2601), (2771-2774), (3149-3152), (3277-3280), (3285-3288), (3654-3657), (3773- |
| | 3776), (3851-3854), (3896-3899), (3947-3950), (3952-3955), (3971-3974), (4048- |
| | 4051), (4062-4065), (4081-4084), (4105-4108), (4405-4408), (4513-4516), (4544- |
| | 4547), (5180-5183), (5272-5275), (5321-5324), (5340-5343), (5406-5409), (5505- |
| | 5508), (5722-5725), (5845-5848), (6169-6172), (6353-6356), (6842-6845), (6919- |
| | 6922), (7022-7025), (7081-7084), (7143-7146), (7188-7191), (7315-7318), (7426- |
| | 7429), (7553-7556), (7718-7721), (7969-7972), (8032-8035), (8171-8174), (8191- |
| | 8194), (8268-8271), (8375-8378), (8521-8524), (8540-8543), (8634-8637), (8643- |
| | 8646), (8771-8774), (8898-8901), (9377-9380), (9430-9433), (9757-9760), (10310- |
| | 10313), (10352-10355), (10367-10370), (10374-10377), (11055-11058), (11160- |
| | 11163), (11463-11466), (11698-11701), (11765-11768), (11779-11782), (11797- |
| | 11800), (12204-12207), (12316-12319), (12355-12358), (12692-12695), (12964- |
| | 12967), (13106-13109), (13137-13140), (13162-13165), (13175-13178), (13200- |
| | 13203), (13360-13363), (13408-13411), (13550-13553), (13555-13558), (13724- |
| | 13727), (14103-14106), (14109-14112), (14119-14122), (14128-14131), (14137- |
| | 14140), (14147-14150), (14214-14217), (14252-14255), (14638-14641), (14684- |
| | 14687), (12204-12207), (12316-12319), (12355-12358), (12692-12695), (12964- |
| | 12967), (13106-13109), (13137-13140), (13162-13165), (13175-13178), (13200- |
| | 13203), (13360-13363), (13408-13411), (13550-13553), (13555-13558), (13724- |
| | 13727), (14103-14106), (14109-14112), (14119-14122), (14128-14131), (14137- |
| | 14140), (14147-14150), (14214-14217), (14252-14255), (14638-14641), (14684- |
| | 14687), (16645-16648), (16889-16892), (16936-16939), (17063-17066), (17470- |
| | 17473), (17735-17738), (17855-17858), (17890-17893), (17956-17959), (18627- |
| | 18630), (18664-18667), (19359-19362), (19767-19770), (19832-19835), (19973- |
| | 19976), (20274-20277), (20522-20525), (20620-20623), (21234-21237), (21382- |
| | 21385), (21525-21528), (21764-21767), (22442-22445), (22535-22538), (22593- |
| | 22596), (22715-22718), (22851-22854), (23055-23058), (23397-23400), (23553- |
| | 23556), (23580-23583), (23588-23591), (23763-23766), (23806-23809), (24190- |
| | 24193), (24294-24297), (24299-24302), (24378-24381), (24601-24604), (24688- |
| | 24691), (24811-24814), (24821-24824), (24843-24846), (24848-24851), (24903- |
| | 24906), (25777-25780), (25859-25862), (25929-25932), (26011-26014), (26322- |
| | 26325), (26334-26337), (26343-26346), (26768-26771), (26959-26962), (27097- |
| | 27100), (27512-27515), (27823-27826), (27940-27943), (28136-28139), (28411- |
| | 28414), (28663-28666), (28926-28929), (29274-29277), (29311-29314), (29483- |
| | 29486), (29494-29497), (29513-29516), (29640-29643), (30478-30481), (30577- |
| | 30580), (30682-30685), (31015-31018), (31032-31035), (31180-31183), (31253- |
| | 31256), (31259-31262), (31264-31267), (31344-31347), (31601-31604), (31812- |
| | 31815), (32045-32048), (32177-32180), (32435-32438), (32881-32884), (32898- |
| | 32901), (32903-32906), (33140-33143), (33233-33236), (33263-33266), (33269- |
| | 33272), (33302-33305), (33325-33328), (33331-33334), (33447-33450), (33464- |
| | 33467), (33529-33532), (33534-33537), (33584-33587), (33606-33609), |
| | (3370533708), (33748-33751), (34033-34036), (34251-34254), (34386-34389), |
| | (34484-34487), (34606-34609), (34758-34761), (34819-34822), (34876-34879), |
| | (35158-35161), (35190-35193), (35286-35289), (35560-35563), (37026-37029), |
| | (37494-37497), (37837-37840), (38088-38091), (38175-38178), (38313-38316), |
| | (38327-38330), (38479-38482), (38808-38811), (38990-38993), (3930439307), |
| | (39378-39381), (39480-39483), (39756-39759), (40160-40163), (40657-40660), |
| | (40926-40929), (40967-40970), (41076-41079), (41427-41430), (41844-41847), |
| | (41871-41874), (42531-42534), (42583-42586), (42684-42687), (42689-42692), |
| | (42694-42697), (42739-42742), (42863-42866), (42928-42931), (43027-43030), |
| | (43239-43242), (43433-43436), (43446-43449), (44143-44146), (44178-44181), |
| | (44270-44273), (44382-44385), (44762-44765), (44881-44884), (45350-45353), |
| | (45672-45675), (45761-45764), (46080-46083), (46169-46172), (46511-46514), |
| | (46722-46725), (46845-46848), (46989-46992), (47029-47032), (47057-47060), |
| | (47155-47158), (47199-47202), (47259-47262), (47359-47362), (47483-47486), |
| | (47833-47836), (48150-48153), (48592-48595), (48624-48627), (48629-48632), |
| | (48700-48703), (48729-48732), (48792-48795), (49438-49441), (50167-50170), |
| | (50221-50224) |
| G₄ | (882-885), (2676-2679), (5438-5441), (8384-8387), (8569-8572), (10290-10293), |
| | (11625-11628), (12119-12122), (12489-12492), (15230-15233), (15883-15886), |
| | (15894-15897), (18592-18595), (18641-18644), (18653-18656), (19418-19421), |
| | (19629-19632), (19687-19690), (19698-19701), (24156-24159), (25211-25214), |
| | (26502-26505), (26631-26634), (28312-28315), (28335-28338), (31574-31577), |
| | (32350-32353), (33697-33700), (36293-36296), (36691-36694), (37168-37171), |
| | (38627-38630), (39075-39078), (41988-41991), (42728-42731), (42971-42974), |
| | (50019-50022) |
| C₄ | (2992-2995), (3124-3127), (3243-3246), (3355-3358), (3996-3999), (4445-4448), |
| | (5527-5530), (6368-6371), (9667-9670), (11489-11492), (11693-11696), (14323- |
| | 14326), (14697-14700), (16808-16811), (17217-17220), (18809-18812), (19599- |
| | 19602), (23063-23066), (24980-24983), (25500-25503), (26507-26510), (26668- |
| | 26671), (27645-27648), (29126-29129), (31789-31792), (34186-34189), (34320- |
| | 34323), (34358-34361), (34587-34590), (36070-36073), (37604-37607), (38609- |
| | 38612), (39159-39162), (40747-40750), (41209-41212), (41500-41503), (41571- |
| | 41574), (45065-45068), (45200-45203), (45290-45293), (45914-45917), (49011- |
| | 49014), (49334-49337) |
| A₅ | (24-28), (534-538), (589-593), (1775-1779), (1984-1988), (2053-2057), (2237- |
| | 2241), (2351-2355), (2855-2859), (2917-2921), (2957-2961), (1620-1624), (2802- |
| | 2806), (3804-3808), (4204-4208), (4927-4931), (5331-5335), (5571-5575), (5615- |
| | 5619), (5873-5877), (6661-6665), (6829-6833), (6861-6865), (7735-7739), (7769- |
| | 7773), (8794-8798), (8833-8837), (9501-9505), (10108-10112), (10985-10989), |
| | (10991-10995), (11546-11550), (11841-11845), (12439-12443), (12861-12865), |
| | (13034-13038), (13041-13045), (13048-13052), (13060-13064), (13285-13289), |
| | (13300-13304), (13427-13431), (13526-13530), (13882-13886), (14626-14630), |
| | (14999-15003), (15103-15107), (15123-15127), (15788-15792), (16206-16210), |
| | (16377-16381), (16464-16468), (16483-16487), (17347-17351), (18219-18223), |
| | (18295-18299), (18669-18673), (18739-18743), (19424-19428), (20187-20191), |
| | (21824-21828), (21879-21883), (22684-22688), (23050-23054), (23155-23159), |
| | (23189-23193), (23413-23417), (24363-24367), (24571-24575), (24698-24702), |
| | (24712-24716), (24898-24902), (25007-25011), (26118-26122), (26260-26264), |
| | (26459-26463), (26533-26537), (26734-26738), (26922-26926), (27060-27064), |
| | (27228-27232), (27379-27383), (27586-27590), (27593-27597), (27723-27727), |
| | (27989-27993), (28011-28015), (30548-30552), (30566-30570), (30617-30621), |
| | (32118-32122), (32200-32204), (32704-32708), (32985-32989), (33095-33099), |
| | (33205-33209), (33548-33552), (33943-33947), (34112-34116), (34624-34628), |
| | (35696-35700), (35738-35742), (35920-35924), (37104-37108), (37426-37430), |
| | (38119-38123), (38130-38134), (38222-38226), (38263-38267), (39265-39269), |
| | (40788-40792), (41171-41175), (41254-41258), (41589-41593), (43114-43118), |
| | (44254-44258), (44307-44311), (44320-44324), (44356-44360), (45257-45261), |
| | (45334-45338), (45600-45604), (46250-46254), (47279-47283), (48098-48102), |
| | (48990-48994), (49141-49145), (49455-49459), (50118-50122), (50147-50151) |
| T₅ | (505-509), (2131-2135), (2188-2192), (2217-2221), (2548-2552), (2617-2621), |
| | (2831-2835), (3371-3375), (3528-3532), (3640-3644), (3932-3936), (4230-4234), |
| | (4270-4274), (6621-6625), (7420-7424), (7432-7436), (7443-7447), (7449-7453), |
| | (7990-7994), (8529-8533), (8653-8657), (9357-9361), (9542-9546), (9559-9563), |
| | (9590-9594), (9600-9604), (9845-9849), (12238-12242), (14242-14246), (15006- |
| | 15010), (16231-16235), (16823-16827), (16833-16837), (18176-18180), (18728- |
| | 18732), (19772-19776), (20290-20294), (20797-20801), (21293-21297), (21648- |
| | 21652), (23288-23292), (23494-23498), (23593-23597), (24037-24041), (26982- |
| | 26986), (25247-25251), (25800-25804), (26762-26766), (29046-29050), (30761- |
| | 30765), (31119-31123), (31300-31304), (31515-31519), (32356-32360), (32669- |
| | 32673), (32800-32804), (32822-32826), (33454-33458), (33620-33624), (34143- |
| | 34147), (34685-34689), (34881-34885), (34973-34977), (35175-35179), (35383- |
| | 35387), (35781-35785), (37359-37363), (37486-37490), (37733-37737), (38303- |
| | 38307), (38731-38735), (38843-38847), (38934-38938), (40614-40618), (41196- |
| | 41200), (41791-41795), (42459-42463), (42675-42679), (43169-43173), (43522- |
| | 43526), (43962-43966), (44334-44338), (46897-46901), (46904-46908), (49382- |
| | 49386), (48325-48329), (48442-48446), (48038-48042) |
| G₅ | (539-543), (667-671), (731-735), (3606-3610), (5048-5052), (5465-5469), (15276- |
| | 15280), (18517-18521), (19795-19799), (28577-28581), (40821-40825), (42471- |
| | 42475), (49718-49722) |
| C₅ | (3087-3091), (5094-5098), (18164-18168), (18236-18240), (18247-18251), (18748- |
| | 18752), (19182-19186), (20410-20414), (24181-24185), (25660-25664), (29118- |
| | 29122), (32500-32504), (41021-41025), (42097-42101), (49299-49303), (49657- |
| | 49661) |
| A₆ | (552-557), (2494-2499), (5781-5786), (6586-6591), (7131-7136), (7348-7353), |
| | (7778-7783), (8145-8150), (9864-9869), (10978-10983), (14886-14891), (17093- |
| | 17098), (17752-17757), (18129-18134), (20572-20577), (20754-20759), (23304- |
| | 23309), (24024-24029), (25306-25311), (26075-26080), (27114-27119), (27859- |
| | 27864), (28582-28587), (29451-29456), (29532-29537), (31397-31402), (31452- |
| | 31457), (37346-37351), (38249-38254), (38452-38457), (40387-40392), (43675- |
| | 43680), (44957-44962), (45079-45084), (45322-45327), (45689-45694), (45736- |
| | 45741), (49150-49155) |
| T₆ | (1327-1332), (1958-1963), (3679-3684), (4525-4530), (6113-6118), (8399-8404), |
| | (9632-9637), (9719-9724), (10116-10121), (11787-11792), (12156-12161), (14905- |
| | 14910), (15304-15309), (19103-19108), (21484-21489), (24826-24831), (25898- |
| | 25903), (26492-26497), (29279-29284), (29442-29447), (31269-31274), (32127- |
| | 32132), (32316-32321), (34069-34074), (34691-34696), (34902-34907), (34981- |
| | 34986), (35196-35201), (37439-37444), (38200-38205), (38374-38379), (38400- |
| | 38405), (38603-38608), (40815-40820), (42628-42633), (43105-43110), (45009- |
| | 45014), (46121-46126), (46327-46332), (46890-46895), (48479-48484), (48521- |
| | 48526), (50231-50236) |
| G₆ | (8065-8070), (24201-24206), (28243-28248) |
| C₆ | (5410-5415), (33839-33844), (40003-40008) |
| A₇ | (874-880), (5248-525411850-11856), (16675-16681), (17481-17487), (23511- |
| | 23517), (27192-27198), (30843-30849), (32215-32221), (33797-33803), (37113- |
| | 37119), (37214-37220), (38662-38668), (38886-38892), (41696-41702), (46551- |
| | 46557), (47780-47786), (50136-50142) |
| T₇ | (2686-2692), (3779-3785), (7625-7631), (14092-14098), (18699-18705), (20139- |
| | 20145), (24524-24530), (28627-28633), (28886-28892), (28961-28967), (33541- |
| | 33547), (38256-38262), (39487-39493), (39764-39770), (42194-42200), (43130- |
| | 43136), (43321-43327) |
| C₇ | (1349-1355), (31644-31650) |
| A₈ | (3913-3920), (6571-6578), (18137-18144), (20597-20604), (27781-27788), (28043- |
| | 28050), (37014-37021), (42983-42990), (43566-43573), (46195-46202), (46223- |
| | 46230) |
| T₈ | (9107-9114), (16839-16846), (27086-27093), (29154-29161), (29355-29362), |
| | (38365-38372) |
| A₉ | (1763-1771), (15289-15297), (40377-40385) |
| T₉ | (9548-9556), (14194-14202), (14794-14802), (25967-25975) |
| A₁₀ | (5256-5265), (20580-20589), (41155-41164) |
| T₁₀ | (39962-39971) |
| A₁₁ | (3686-3696), (49391-49401) |
| T₁₂ | (7997-8008) |
| A₁₃ | (11908-11920) |
| T₁₅ | (7115-7129) |
| A₁₈ | (20694-20711) |
| (AC)₄ | (40080-40087) |
| (AG)₄ | (49705-49712) |
| (AT)₄ | (3728-3735), (8969-8976), (16694-16701), (35023-35030), (39183-39190), (50110- |
| | 50117) |
| (CT)₄ | (18893-18900), (25457-25464), (32054-32061), (34062-34069), (40196-40203) |
| (TA)₄ | (9272-9279), (16083-16090), (35332-35339), (42565-42572), (46626-46633) |
| (TG)₄ | (36186-36193) |
| (TC)₄ | (37497-37504) |
| (AC)₅ | (34995-35004) |
| (AT)₅ | (29759-29768) |
| (CT)₅ | (37457-37466) |
| (TC)₅ | (18880-18889), (18904-18913), (25438-25447) |
| (TG)₅ | (46608-46617) |
| (GA)₅ | (41263-41272) |
| (GA)₆ | (18521-18532 |
| (CT)₇ | (18103-18116) |
| (TA)₇ | (30405-30418) |
| (AG)₉ | (16020-16037) |
| (TA)₁₁ | (30913-30934) |
| (TA)₁₅ | (35048-35077) |
| (TAA)₄ | (2156-2167) |
| (TCA)₄ | (19108-19119) |
| (GAG)₄ | (19303-19314) |
| (AAT)₄ | (35671-35682) |
| (GGT)₅ | (49757-49771) |
| (GAGG)₃ | (24531-24542) |
| (CATG)₃ | (29691-29702) |
| (CAAG)₃ | (41276-41287) |
| (GTGC)₄ | (35349-35364) |
| (TATC)₆ | (30889-30912) |
| (ATAC)₈ | (29660-29693) |
| (TATC)₈ | (30979-31010) |
| (GACCC)₃ | (25280-25294) |

The SSRs identified in the PDK_{—}30s1202771 scaffold (GenBank accession number: GL744456-20074 nucleotides in length) and their respective locations in this scaffold are given in the following table:

| SSR | Positions occupées dans le scaffold PDK_30s1202771 |
|---|---|
| A₄ | (1108-1111), (1117-1120), (1359-1362), (2302-2305), (2314-2317), (2457-2460), |
| | (2639-2642), (2887-2890), (2987-2990), (3113-3116), (3148-3151), (3361-3364), |
| | (3385-3388), (3508-3511), (3568-3571), (3751-3754), (3893-3896), (4092-4095), |
| | (4210-4213), (4333-4336), (4607-4610), (4870-4873), (5236-5239), (5266-5269), |
| | (5291-5294), (5411-5414), (5657-5660), (5969-5972), (6345-6348), (6686-6689), |
| | (6946-6949), (7188-7191), (7208-7211), (7388-7391), (7616-7619), (7712-7715), |
| | (8592-8595), (8615-8618), (9020-9023), (9718-9721), (11479-11482), (12076- |
| | 12079), (12046-12049), (12878-12881), (13075-13078), (13134-13137), (13181- |
| | 13184), (13257-13260), (13563-13566), (13502-13505), (13781-13784), (13881- |
| | 13884), (13947-13950), (14282-14285), (14300-14303), (14453-14456), (14685- |
| | 14688), (14807-14810), (14955-14958), (15787-15790), (16191-16194), (16242- |
| | 16245), (16250-16253), (16318-16321), (16511-16514), (16891-16894), (16905- |
| | 16908), (16918-16921), (16981-16984), (17028-17031), (17037-17040), (17301- |
| | 17304), (18970-18973), (19393-19396), (19411-19414), (19999-20002) |
| T₄ | (187-190), (1194-1197), (1261-1264), (1659-1662), (1694-1697), (2226-2229), |
| | (2347-2350), (2444-2447), (2449-2452), (2730-2733), (2952-2955), (3135-3138), |
| | (3153-3156), (3467-3470), (3610-3613), (3646-3649), (4050-4053), (4129-4132), |
| | (4242-4245), (4255-4258), (4341-4344), (5184-5187), (5389-5392), (5532-5535), |
| | (5687-5690), (6321-6324), (6459-6462), (6662-6665), (6754-6757), (6903-6906), |
| | (7032-7035), (7050-7053), (7170-7173), (7256-7259), (7463-7466), (7548-7551), |
| | (7572-7575), (7831-7834), (7851-7854), (7974-7977), (8215-8218), (8448-8451), |
| | (8518-8521), (8558-8561), (8619-8622), (8655-8658), (8834-8837), (8846-8849), |
| | (8869-8872), (9050-9053), (9274-9277), (9308-9311), (9648-9651), (9870-9873), |
| | (10939-10942), (10980-10983), (10994-10997), (11409-11412), (11464-11467), |
| | (11505-11508), (12601-12604), (12858-12861), (12942-12945), (13725-13728), |
| | (13747-13750), (13846-13849), (13988-13991), (15113-15116), (15573-15576), |
| | (15581-15584), (16103-16106), (16593-16596), (16691-16694), (17226-17229), |
| | (17278-17281), (19657-19660), (19772-19775), (19861-19864) |
| G₄ | (1275-1278), (1303-1306), (2198-2201), (4795-4798), (5806-5809), (8541-8544), |
| | (10900-10903), (11012-11015), (11017-11020), (11036-11039), (11210-11213), |
| | (11317-11320), (11377-11380), (11864-11867), (12326-12329), (12373-12376), |
| | (12629-12632), (14366-14369), (15133-15136), (16678-16681), (16767-16770), |
| | (19127-19130), (19494-19497), (19557-19560), (19740-19743), (20042-20045) |
| C₄ | (194-197), (2677-2680), (9524-9527), (10324-10327), (11239-11242), (11783- |
| | 11786), (14079-14082), (14101-14104), (14118-14121), (14595-14598), (14629- |
| | 14632), (14903-14906), (15028-15031), (15201-15204), (15344-15347), (17424- |
| | 17427), (18866-18869), (19014-19017), (19053-19056), (19147-19150), (19212- |
| | 19215), (19567-19570), (19928-19931) |
| A₅ | (986-990), (1159-11631189-1193), (1618-1622), (2048-2052), (2526-2530), (2749- |
| | 2753), (3472-3476), (3496-3500), (4357-4361), (4500-4504), (5730-5734), (6416- |
| | 6420), (6837-6841), (7103-7107), (7513-7517), (8828-8832), (11484-11488), |
| | (12154-12158), (12795-12799), (12980-12984), (13140-13144), (13520-13524), |
| | (13606-13610), (14036-14040), (14049-14053), (15241-15245), (16098-16102), |
| | (16266-16270), (16272-16276), (16845-16849), (16861-16865), (17202-17206) |
| T₅ | (1015-1019), (1201-1205), (1997-2001), (3818-3822), (4069-4073), (4187-4191), |
| | (4372-4376), (4988-4992), (5512-5516), (5612-5616), (5643-5647), (6177-6181), |
| | (7399-7403), (7684-7688), (7773-7777), (8045-8049), (8342-8346), (8529-8533), |
| | (8874-8878), (8893-8897), (9681-9685), (10230-10234), (11078-11082), (11165- |
| | 11169), (12137-12141), (13020-13024), (13055-13059), (13460-13464), (13554- |
| | 13558), (13705-13709), (13712-13716), (15510-15514), (16786-16790), (16895- |
| | 16899), (16949-16953) |
| G₅ | (40-44), (11097-11101), (16493-16497) |
| C₅ | (10904-10908), (10951-10955), (10957-10961), (14149-14153), (15015-15019), |
| | (17342-17346), (19500-19504) |
| A₆ | (26-31), (2205-2210), (7766-7771), (12999-13004), (15300-15305), (16278-16283) |
| T₆ | (1308-1313), (5194-5199), (7607-7612), (8640-8645), (9622-9627), (12913- |
| | 12918), (13718-13723) |
| G₆ | (11065-11070) |
| C₆ | (10555-10560), (19159-19164), (19795-19800) |
| A₇ | (1099-1105), (2755-2761), (7521-7527), (15177-15183), (16038-16044) |
| T₇ | (2255-2261), (6676-6682), (8122-8128), (8931-8937), (10676-10682) |
| A₈ | (9556-9563), (11470-11477) |
| T₈ | (2554-2561) |
| T₉ | (5482-5490), (12968-12976) |
| T₁₀ | (3620-3629) |
| T₁₂ | (13961-13972) |
| A₁₇ | (17055-17071) |
| (AT)₄ | (1790-1797), (11297-11304) |
| (GA)₄ | (11380-11387), (12867-12874) |
| (TC)₄ | (4305-4312), (15834-15841) |
| (CA)₄ | (14702-147090 |
| (CA)₅ | (5860-5869) |
| (CG)₇ | (5846-5859) |
| (AT)₇ | (7063-7076) |
| (GA)₁₀ | (5871-5890) |
| (CATG)₃ | (15556-15567) |

The SSRs identified in the PDK_{—}30s6550963 scaffold (GenBank accession number: GL739764-95115 nucleotides in length) and their respective locations in this scaffold are given in the following table:

| SSR | Positions occupees dans le scaffold PDK_30s6550963 |
|---|---|
| A₄ | (63-66), (316-319), (322-325), (316-319), (322-325), (496-499), (530-533), (570- |
| | 573), (703-706), (735-738), (1389-1392), (1430-1433), (1274-1277), (1521-1524), |
| | (1575-1578), (1807-1810), (2010-2013), (2100-2103), (2306-2309), (2574-2577), |
| | (3767-3770), (3803-3806), (3839-3842), (3869-3872), (3921-3924), (4526-4529), |
| | (4567-4570), (4818-4821), (5611-5614), (5531-5534), (6023-6026), (6105-6108), |
| | (6192-6195), (6508-6511), (6608-6611), (6888-6891), (6917-6920), (7195-7198), |
| | (7606-7609), (8098-8101), (8469-8472), (8564-8567), (8700-8703), (8846-8849), |
| | (8870-8873), (8945-8948), (9113-9116), (9124-9127), (9149-9152), (9170-9173), |
| | (9290-9293), (9479-9482), (9614-9617), (9644-9647), (9739-9742), (9972-9975), |
| | (9983-9986), (10358-10361), (10778-10781), (10880-10883), (10961-10964), |
| | (11177-11180), (11417-11420), (11757-11760), (12065-12068), (12381-12384), |
| | (12423-12426), (12542-12545), (12635-12638), (12945-12948), (12965-12968), |
| | (13870-13873), (14194-14197), (14003-14006), (14478-14481), (14485-14488), |
| | (15226-15229), (15264-15267), (15470-15473), (15964-15967), (16052-16055), |
| | (16258-16261), (16303-16306), (16576-16579), (16675-16678), (17349-17352), |
| | (17392-17395), (17640-17643), (17652-17655), (17686-17689), (17716-17719), |
| | (17765-17768), (17845-17848), (18347-18350), (18530-18533), (18652-18655), |
| | (18889-18892), (19171-19174), (19296-19299), (19318-19321), (19452-19455), |
| | (19683-19686), (19901-19904), (19928-19931), (19990-19993), (20038-20041), |
| | (20081-20084), (20264-20267), (20596-20599), (20749-20752), (21121-21124), |
| | (21459-21462), (21494-21497), (21518-21521), (21569-21572), (22753-22756), |
| | (22881-22884), (22978-22981), (23018-23021), (23160-23163), (23243-23246), |
| | (23273-23276), (23445-23448), (23617-23620), (23622-23625), (23631-23634), |
| | (23706-23709), (23890-23893), (24148-24151), (24166-24169), (24279-24282), |
| | (24384-24387), (24461-24464), (24552-24555), (25962-25965), (26034-26037), |
| | (26055-26058), (26106-26109), (26255-26258), (26268-26271), (26321-26324), |
| | (26386-26389), (26410-26413), (26527-26530), (26665-26668), (26695-26698), |
| | (26719-26722), (27576-27579), (27605-27608), (27637-27640), (27691-27694), |
| | (27699-27702), (27752-27755), (27781-27784), (27910-27913), (27942-27945), |
| | (27975-27978), (28115-28118), (28148-28151), (28192-28195), (28259-28262), |
| | (28307-28310), (28352-28355), (28616-28619), (28736-28739), (28802-28805), |
| | (29273-29276), (29326-29329), (29365-29368), (29407-29410), (29413-29416), |
| | (29442-29445), (29557-29560), (29617-29620), (29741-29744), (30007-30010), |
| | (30095-30098), (30102-30105), (30116-30119), (30188-30191), (30198-30201), |
| | (30278-30281), (30416-30419), (30467-30470), (30609-30612), (30636-30639), |
| | (30706-30709), (30724-30727), (30766-30769), (30878-30881), (30980-30983), |
| | (31015-31018), (31023-31026), (31099-31102), (31216-31219), (31347-31350), |
| | (31476-31479), (31513-31516), (31820-31823), (31906-31909), (32061-32064), |
| | (32107-32110), (32226-32229), (32402-32405), (32535-32538), (32886-32889), |
| | (32950-32953), (33170-33173), (33176-33179), (33319-33322), (33330-33333), |
| | (33340-33343), (34472-34475), (34609-34612), (34682-34685), (34766-34769), |
| | (34792-34795), (34807-34810), (34903-34906), (35124-35127), (35166-35169), |
| | (35220-35223), (35247-35250), (35282-35285), (35287-35290), (35445-35448), |
| | (35589-35592), (35595-35598), (35797-35800), (36409-36416), (36500-36503), |
| | (36614-36617), (36784-36787), (36799-36802), (37585-37588), (37590-37593), |
| | (37729-37732), (37826-37829), (38146-38149), (38591-38594), (38608-38611), |
| | (38781-38784), (38855-38858), (38875-38878), (38910-38913), (39029-39032), |
| | (39127-39130), ( ), (39152-39155), (39311-39314), (39419-39422), (39515-39518), |
| | (39556-39559), (39677-39680), (39832-39835), (39857-39860), (39947-39950), |
| | (40052-40055), (40086-40089), (40129-40132), (40145-40148), (40253-40256), |
| | (40401-40404), (40602-40605), (40746-40749), (40808-40811), (40885-40888), |
| | (40892-40895), (40953-40956), (40972-40975), (41242-41245), (41281-41284), |
| | (41361-41364), (41394-41397), (41400-41403), (41576-41579), (41659-41662), |
| | (41672-41675), (41425-41428), (41438-41441), (41510-41513), (42988-42991), |
| | (43560-43563), (43781-43784), (43890-43893), (44031-44034), (44134-44137), |
| | (44318-44321), (44598-44601), (44734-44737), (44739-44743), (44864-44867), |
| | (45136-45139), (45167-45170), (45174-45177), (45625-45628), (45765-45768), |
| | (45797-45800), (45854-45857), (45859-45862), (45928-45931), (45985-45988), |
| | (46099-46102), (46108-46111), (46183-46186), (46254-46257), (46259-46262), |
| | (46283-46286), (46376-46379), (46516-46519), (46525-46528), (46534-46537), |
| | (46540-46543), (46557-46560), (46568-46571), (46875-46878), (47087-47090), |
| | (47363-47366), (47795-47798), (47842-47845), (47906-47909), (48224-48227), |
| | (48512-48515), (48940-48943), (48971-48974), (49527-49530), (49567-49570), |
| | (49766-49769), (49778-49781), (50197-50200), (50287-50290), (50526-50529), |
| | (50531-50534), (50713-50716), (51104-51107), (51246-51249), (51386-51389), |
| | (51408-51411), (51493-51496), (51790-51793), (51861-51864), (51910-51913), |
| | (52610-52613), (53231-53234), (53628-53631), (53873-53876), (54068-54071), |
| | (54083-54086), (54118-54121), (54218-54221), (54263-54266), (54655-54658), |
| | (54956-54959), (55274-55277), (55522-55525), (55555-55558), (55711-55714), |
| | (55872-55875), (55926-55929), (56011-56014), (56258-56261), (56333-56336), |
| | (56385-56388), (56428-56431), (56416-56419), (56557-56560), (56579-56582), |
| | (56591-56594), (56637-56640), (56733-56736), (56743-56746), (56805-56808), |
| | (56829-56832), (56963-56966), (58107-58110), (58167-58170), (58844-58847), |
| | (58901-58904), (59005-59008), (59029-59032), (59140-59143), (59176-59179), |
| | (59283-59286), (59542-59545), (59638-59641), (59798-59801), (59925-59928), |
| | (59972-59975), (60471-60474), (60510-60513), (60598-60601), (60736-60739), |
| | (60818-60821), (61251-61254), (61308-61311), (61367-61370), (61420-61423), |
| | (61517-61520), (62379-62382), (62765-62768), (62800-62803), (62920-62923), |
| | (63256-63259), (63438-63441), (63585-63588), (64049-64052), (64608-64611), |
| | (64854-64857), (64869-64872), (64946-64949), (65022-65025), (65159-65162), |
| | (65182-65185), (65416-65419), (65554-65557), (65628-65631), (65679-65682), |
| | (65718-65721), (66471-66474), (66624-66627), (66629-66632), (66654-66657), |
| | (66731-66734), (67293-67296), (67377-67380), (67405-67408), (67661-67664), |
| | (67674-67677), (67821-67824), (68352-68355), (68421-68424), (68497-68500), |
| | (68711-68714), (68725-68728), (68783-68786), (68824-68827), (69350-69353), |
| | (69510-69513), (69772-69775), (70125-70128), (70217-70220), (70224-70227), |
| | (70348-70351), (70432-70435), (70613-70616), (70951-70954), (71362-71365), |
| | (71452-71455), (71628-71631), (71666-71669), (71713-71716), (71860-71863), |
| | (72275-72278), (72350-72353), (72377-72380), (72384-72387), (74112-74115), |
| | (74127-74130), (74157-74160), (74838-74841), (74857-74860), (75067-75070), |
| | (75171-75174), (75365-75368), (75475-75478), (75736-75739), (75773-75776), |
| | (76037-76040), (76046-76049), (76076-76079), (76192-76195), (76389-76392), |
| | (76534-76537), (76542-76545), (76652-76655), (76668-76671), (76687-76690), |
| | (76928-76931), (79325-79328), (79519-79522), (79627-79630), (79655-79658), |
| | (79736-79739), (79930-79933), (80366-80369), (80878-80881), (80897-80900), |
| | (81091-81094), (81825-81828), (81974-81977), (81987-81990), (82075-82078), |
| | (82181-82184), (84349-84352), (84614-84617), (84711-84714), (84924-84927), |
| | (85665-85668), (86037-86040), (86084-86087), (86092-86095), (86702-86705), |
| | (87142-87145), (87152-87155), (87164-87167), (87255-87258), (87350-87353), |
| | (88784-88787), (89052-89055), (89116-89119), (89181-89184), (89305-89308), |
| | (89353-89356), (89414-89417), (89441-89444), (89545-89548), (89663-89666), |
| | (89679-89682), (89753-89756), (89784-89787), (90233-90236), (90324-90327), |
| | (90337-90340), (91037-91040), (91046-91049), (92464-92467), (92530-92533), |
| | (92677-92680), (93570-93573), (93720-93723), (93974-93977), (94036-94039), |
| | (94081-94084), (94178-94181), (94389-94392), (94882-94885), (94948-94951), |
| | (95034-95037), (95050-95053), (95084-95087) |
| T₄ | (68-71), (117-120), (617-620), (1069-1072), (1441-1444), (1553-1556), (1569- |
| | 1572), (1709-1712), (1741-1744), (2169-2172), (2194-2197), (2014-2017), (2112- |
| | 2115), (2639-2642), (2644-2647), (2668-2671), (2762-2765), (2852-2855), (3015- |
| | 3018), (3324-3327), (4076-4079), (4353-4356), (4469-4472), (4782-4785), (4809- |
| | 4812), (4833-4836), (4842-4845), (5645-5648), (6199-6202), (6457-6460), (6482- |
| | 6485), (8048-8051), (8127-8130), (8318-8321), (8364-8367), (8480-8483), (8545- |
| | 8548), (8600-8603), (8850-8853), (9078-9081), (9295-9298), (9344-9347), (9560- |
| | 9563), (10443-10446), (10459-10462), (10464-10467), (11366-11369), (11421- |
| | 11424), (11551-11554), (11941-11944), (12022-12025), (12404-12407), (12492- |
| | 12495), (12516-12519), (12688-12691), (12750-12753), (12980-12983), (13796- |
| | 13799), (14112-14115), (14222-14225), (15206-15209), (15216-15219), (15508- |
| | 15511), (15710-15713), (15723-15726), (15884-15887), (16191-16194), (16497- |
| | 16500), (16535-16538), (16589-16592), (16615-16618), (16659-16662), (16712- |
| | 16715), (16757-16760), (16815-16818), (16929-16932), (17094-17097), (17245- |
| | 17248), (17291-17294), (17361-17364), (17369-17372), (17374-17377), (17488- |
| | 17491), (17548-17551), (17634-17637), (17960-17963), (18248-18251), (18486- |
| | 18489), (18526-18529), (18640-18643), (19313-19316), (19440-19443), (19604- |
| | 19607), (19881-19884), (19952-19955), (20075-20078), (20099-20102), (20125- |
| | 20128), (20234-20237), (20363-20366), (20448-20451), (20530-20533), (20788- |
| | 20791), (20831-20834), (20874-20877), (21843-21846), (21923-21926), (22984- |
| | 22987), (23045-23048), (23086-23089), (23097-23100), (23106-23109), (23385- |
| | 23388), (23454-23457), (24472-24475), (24667-24670), (24902-24905), (25015- |
| | 25018), (25131-25134), (25381-25384), (25443-25446), (25655-25658), (25849- |
| | 25852), (25914-25917), (25993-25996), (26061-26064), (27145-27148), (27560- |
| | 27563), (27765-27768), (28064-28067), (28230-28233), (28248-28251), (28344- |
| | 28347), (28371-28374), (28512-28515), (28740-28743), (28882-28885), (28887- |
| | 28890), (28990-28993), (28997-29000), (29030-29033), (29621-29624), (29814- |
| | 29817), (29954-29957), (30239-30242), (30582-30585), (30591-30594), (30743- |
| | 30746), (30752-30755), (30906-30909), (31042-31045), (31222-31225), (31333- |
| | 31336), (31742-31745), (31879-31882), (31953-31956), (32003-32006), (32020- |
| | 32023), (32353-32356), (32394-32397), (32479-32482), (32729-32732), (32751- |
| | 32754), (33197-33200), (33248-33251), (33409-33412), (33678-33681), (34441- |
| | 34444), (34755-34758), (34864-34867), (34921-34924), (34957-34960), (35054- |
| | 35057), (35255-35258), (35278-35281), (35344-35347), (35355-35358), (36342- |
| | 36345), (36355-36358), (36397-36400), (36449-36452), (36525-36528), (36586- |
| | 36589), (36920-36923), (37190-37193), (37266-37269), (37303-37306), (37667- |
| | 37670), (37758-37761), (37888-37891), (38003-38006), (38096-38099), (38559- |
| | 38562), (38725-38728), (39402-39405), (39407-39410), (39423-39426), (39525- |
| | 39528), (39584-39587), (39656-39659), (39672-39675), (40384-40387), (40555- |
| | 40558), (40561-40564), (40699-40702), (40989-40992), (41982-41985), (41990- |
| | 41993), (42074-42077), (42089-42092), (42343-42346), (42350-42353), (42367- |
| | 42370), (42409-42412), (42497-42500), (42610-42613), (42657-42660), (42721- |
| | 42724), (43129-43132), (43185-43188), (43976-43979), (44340-44343), (44409- |
| | 44412), (44611-44614), (44698-44701), (44774-44777), (44784-44787), (44921- |
| | 44924), (45239-45242), (45374-45377), (45426-45429), (45541-45544), (45801- |
| | 45804), (46187-46190), (46229-46232), (46248-46251), (46318-46321), (46512- |
| | 46515), (46796-46799), (47788-47791), (47835-47838), (48150-48153), (48257- |
| | 48260), (48537-48540), (49049-49052), (49589-49592), (49748-49751), (50123- |
| | 50126), (50327-50330), (50441-50444), (50508-50511), (50768-50771), (50862- |
| | 50865), (51234-51237), (51250-51253), (51273-51276), (51281-51284), (51337- |
| | 51340), (52181-52184), (52256-52259), (52267-52270), (52372-52375), (52856- |
| | 52859), (52879-52882), (52943-52946), (53312-53315), (53330-53333), (53463- |
| | 53466), (53624-53627), (53960-53963), (54012-54015), (54129-54132), (54158- |
| | 54161), (54182-54185), (54322-54325), (54382-54385), (54456-54459), (54841- |
| | 54844), (54995-54998), (55250-55253), (55279-55282), (55319-55322), (55376- |
| | 55379), (55448-55451), (55486-55489), (55716-55719), (55777-55780), (55889- |
| | 55892), (56173-56176), (56230-56233), (56305-56308), (56493-56496), (56503- |
| | 56506), (56540-56543), (56563-56566), (56629-56632), (56710-56713), (56888- |
| | 56891), (56991-56994), (57011-57014), (57037-57040), (57171-57174), (57785- |
| | 57788), (57794-57797), (58008-58011), (58028-58031), (58057-58060), (58147- |
| | 58150), (58163-58166), (58196-58199), (58338-58341), (58826-58829), (58868- |
| | 58871), (58912-58915), (58975-58978), (59136-59139), (59655-59658), (59785- |
| | 59788), (59911-59914), (59962-59965), (59985-59988), (60111-60114), (60555- |
| | 60558), (60590-60593), (60848-60851), (60853-60856), (60917-60920), (60997- |
| | 61000), (61267-61270), (61332-61335), (61858-61861), (61867-61870), (61919- |
| | 61922), (62172-62175), (62332-62335), (62345-62348), (62394-62397), (62448- |
| | 62451), (62503-62506), (62509-62512), (62863-62866), (62924-62927), (63567- |
| | 63570), (63662-63665), (63717-63720), (63773-63776), (63812-63815), (63951- |
| | 63954), (64089-64092), (64196-64199), (64517-64520), (64584-64587), (64816- |
| | 64819), (64865-64868), (64932-64935), (65012-65015), (65045-65048), (65367- |
| | 65370), (66096-66099), (66352-66355), (66586-66589), (66705-66708), (66791- |
| | 66794), (66895-66898), (67236-67239), (67257-67260), (67471-67474), (67615- |
| | 67618), (67649-67652), (67889-67892), (68119-68122), (68133-68136), (68247- |
| | 68250), (68252-68255), (68632-68635), (68639-68642), (68776-68779), (68940- |
| | 68943), (68985-68988), (69022-69025), (69082-69085), (69107-69110), (69372- |
| | 69375), (69514-69517), (69531-69534), (69593-69596), (69780-69783), (69810- |
| | 69813), (69835-69838), (69961-69964), (70317-70320), (70783-70786), (70912- |
| | 70915), (70989-70992), (71069-71072), (71196-71199), (71243-71246), (71342- |
| | 71345), (71347-71350), (71552-71555), (71807-71810), (71815-71818), (71821- |
| | 71824), (72079-72082), (72170-72173), (72289-72292), (72425-72428), (72443- |
| | 72446), (72693-72696), (72880-72883), (72990-72993), (73386-73389), (74063- |
| | 74066), (74107-74110), (74206-74209), (74373-74376), (74459-74462), (74618- |
| | 74621), (75080-75083), (75413-75416), (75427-75430), (75447-75450), (75467- |
| | 75470), (75498-75501), (75638-75641), (75674-75677), (75713-75716), (75729- |
| | 75732), (75777-75780), (75870-75873), (75898-75901), (75923-75926), (76225- |
| | 76228), (76249-76252), (76393-76396), (79422-79425), (79591-79594), (79910- |
| | 79913), (80644-80647), (80801-80804), (81776-81779), (81782-81785), (81866- |
| | 81869), (81895-81898), (81993-81996), (82419-82422), (83438-83441), (83567- |
| | 83570), (83781-83784), (83857-83860), (83894-83897), (84345-84348), (84648- |
| | 84651), (84958-84961), (85884-85887), (86178-86181), (86454-86457), (86791- |
| | 86794), (86807-86810), (87125-87128), (87191-87194), (87209-87212), (87215- |
| | 87218), (88812-88815), (88837-88840), (88954-88957), (90419-90422), (90740- |
| | 90743), (89357-89360), (89398-89401), (89650-89653), (89731-89734), (89821- |
| | 89824), (93503-93506), (94169-94172), (94291-94294), (94472-94475), (94595- |
| | 94598), (94677-94680), (95018-95021), (95059-95062) |
| G₄ | (429-4432), (4500-4503), (6717-6720), (7435-7438), (10495-10498), (10808- |
| | 10811), (11040-11043), (11830-11833), (12036-12039), (12520-12523), (14305- |
| | 14308), (16980-16983), (20567-20570), (20653-20656), (22023-22026), (22526- |
| | 22529), (23726-23729), (24803-24806), (26968-26971), (28943-28946), (33667- |
| | 33670), (33845-33848), (34210-34213), (35724-35727), (35793-35796), (36026- |
| | 36029), (37015-37018), (37376-37379), (37479-37482), (38798-38801), (38826- |
| | 38829), (39175-39178), (40155-40158), (42035-42038), (42193-42196), (42663- |
| | 42666), (44361-44364), (49861-49864), (51703-51706), (51715-51718), (51808- |
| | 51811), (52190-52193), (52623-52626), (55035-55038), (55091-55094), (58544- |
| | 58547), (58642-58645), (58812-58815), (59661-59664), (60180-60183), (61434- |
| | 61437), (61718-61721), (61741-61744), (61854-61857), (62491-62494), (62981- |
| | 62984), (63514-63517), (69472-69475), (69877-69880), (70772-70775), (71151- |
| | 71154), (71717-71720), (71750-71753), (73429-73432), (73823-73826), (74318- |
| | 74321), (74649-74652), (74656-74659), (74730-74733), (75312-75315), (76064- |
| | 76067), ( ), (81843-81846), (82400-82403), (82700-82703), (82841-82844), |
| | (82847-82850), (82853-82856), (83467-83470), (83617-83620), (84110-84113), |
| | (84142-84145), (84333-84336), (84447-84450), (84545-84548), (89475-89478), |
| | (90315-90318), (91186-91189), (91234-91237), (92333-92336), (92712-92715), |
| | (92784-92787), (92856-92859), (92991-92994), (93033-93036), (93129-93132), |
| | (93144-93147), (93330-93333), (93335-93338), (93380-93383), (94467-94470), |
| | (94631-94634), (84757-84760), (84855-84858), (85152-85155), (85544-85547) |
| C₄ | (2139-2142), (3752-3755), (4144-4147), (4723-4726), (5850-5853), (9452-9455), |
| | (12351-12354), (12592-12595), (13880-13883), (14492-14495), (16708-16711), |
| | (17117-17120), (17945-17948), (18287-18290), (22230-22233), (22244-22247), |
| | (23291-23294), (23224-23227), (23766-23769), (24879-24882), (25234-25237), |
| | (25760-25763), (25799-25802), (26365-26368), (29678-29681), (30632-30635), |
| | (31673-31676), (31706-31709), (32867-32870), (33488-33491), (36969-36972), |
| | (38762-38765), (41210-41213), (41838-41841), (43140-43143), (43609-43612), |
| | (43689-43692), (43945-43948), (45273-45276), (45637-45640), ( ), (46835-46838), |
| | (47403-47406), (47628-47631), (48975-48978), (49939-49942), (52349-52352), |
| | (53086-53089), ( ), (53308-53311), (5483054833), (61612-61615), (62120-62123), |
| | (63521-63524), (65436-65439), (67205-67208), (68278-68281), (69926-69929), |
| | (70070-70073), (70155-70158), (70458-70461), (70645-70648), (71353-71356), |
| | (71780-71783), (72798-72801), (74368-74371), (74779-74782), (7509975102), |
| | (76355-76358), (79670-79673), (80767-80770), (82222-82225), (82788-82791), |
| | (82826-82829), (82876-82879), (82945-82948), (82958-82961), (82969-82972), |
| | (83178-83181), (83537-83540), (84239-84242), (84358-84361), (86525-86528), |
| | (86847-86850), (86853-86856), (88609-88612), (89769-89772), (93166-93169), |
| | (93294-93297), (93865-93868), (93897-93900) |
| A₅ | (180-184), (355-359), (515-519), (1475-1479), (1503-1507), (1683-1687), (2327- |
| | 2331), (2457-2461), (1988-1992), (3911-3915), (4777-4781), (6776-6780), (6836- |
| | 6840), (7670-7674), (9161-9165), (9784-9788), (10431-10435), (10482-10486), |
| | (10610-10614), (10620-10624), (10631-10635), (10967-10971), (11470-11474), |
| | (12910-12914), (14528-14532), (15244-15248), (15269-15273), (15289-15293), |
| | (15763-15767), (17240-17244), (17286-17290), (18591-18595), (19397-19401), |
| | (19475-19479), (19611-19615), (22715-22719), (23035-23039), (23057-23061), |
| | (23778-23782), (24056-24060), (24270-24274), (24649-24653), (24853-24857), |
| | (25043-25047), (25049-25053), (25055-25059), (25389-25393), (25399-25403), |
| | (25488-25492), (26210-26214), (26245-26249), (26487-26491), (26738-26742), |
| | (27545-27549), (27594-27598), (27744-27748), (28670-28674), (28751-28755), |
| | (28794-28798), (28850-28854), (28856-28860), (28948-28952), (29239-29243), |
| | (29488-29492), (29958-29962), (30402-30406), (31064-31068), (31579-31583), |
| | (32980-32984), (32986-32990), (32993-32997), (34445-34449), (34889-34893), |
| | (34978-34982), (35026-35030), (35150-35154), (35412-35416), (36289-36293), |
| | (36327-36331), (36549-36553), (36595-36599), (37492-37496), (37942-37946), |
| | (37965-37969), (38881-38885), (38978-38982), (39004-39008), (40311-40315), |
| | (40801-40805), (41688-41692), (44286-44290), (44057-44061), (44928-44932), |
| | (45250-45254), (45499-45503), (46117-46121), (46138-46142), (46392-46396), |
| | (46579-46583), (46603-46607), (47130-47134), (48469-48473), (50026-50030), |
| | (51394-51398), (52031-52035), (53236-53240), (54252-54256), (54615-54619), |
| | (56206-56210), (56212-56216), (56281-56285), (56287-56291), (55996-56000), |
| | (56408-56412), (56454-56458), (56699-56703), (56822-56826), (56834-56838), |
| | (56892-56896), (56917-56921), (56995-56999), (57175-57179), (57834-57838), |
| | (58295-58299), (59052-59056), (59131-59135), (59338-59342), (59946-59950), |
| | (60541-60545), (60730-60734), (62441-62445), (64925-64929), (65575-65579), |
| | (65684-65688), (66808-66812), (67398-67402), (67970-67974), (68068-68072), |
| | (68259-68263), (68393-68397), (68415-68419), (68491-68495), (70688-70692), |
| | (70701-70705), (70731-70735), (72585-72589), (72688-72692), (72942-72946), |
| | (72974-72978), (73972-73976), (74917-74921), (75334-75338), (76641-76645), |
| | (77033-77037), (79727-79731), (79840-79844), (80429-80433), (81980-81984), |
| | (84164-84168), (85614-85618), (85829-85833), (86195-86199), (87035-87039), |
| | (90000-90004), (90055-90059), (90091-90095), (90563-90567), (90610-90614), |
| | (90934-90938), (93425-93429), (94135-94139), (95073-95077) |
| G₅ | (23371-23375), (26961-26965), (34193-34197), (35736-35740), (35943-35947), |
| | (37027-37031), (37739-37743), (38745-38749), (46827-46831), (48822-48826), |
| | (51081-51085), (55085-55089), (65102-65106), (73610-73614), (74450-74454), |
| | (76199-76203), (82642-82646), (83322-83326), (85045-85049), (85224-85228), |
| | (87018-87022), (87105-87109), (91204-91208), (91279-91283), (91294-91298), |
| | (91339-91343), (92151-92155), (92981-92985), (92996-93000), (93062-93066), |
| | (93345-93349), (93403-93407) |
| C₅ | (2611-2615), (3328-3332), (5929-5933), (6465-6469), (8757-8761), (11535- |
| | 11539), (18149-18153), (22214-22218), (22220-22224), (22316-22320), (51286- |
| | 51290), (57025-57029), (62513-62517), (70558-70562), (80089-80093), (83193- |
| | 83197) |
| A₆ | (1116-1121), (2024-2029), (3052-3057), (4504-4509), (8381-8386), (8388-8393), |
| | (8971-8976), (9575-9580), (19793-19798), (19969-19974), (19886-19891), |
| | (23067-23072), (23151-23156), (25645-25650), (26862-26867), (29039-29044), |
| | (27265-27270), (29291-29296), (30442-30447), (31938-31943), (32973-32978), |
| | (34421-34426), (37608-37613), (37720-37725), (39015-39020), (39039-39044), |
| | (39063-39068), (41516-41521), (42970-42975), (43796-43801), (46880-46885), |
| | (47321-47326), (47443-47448), (48217-48222), (51024-51029), (51065-51070), |
| | (54076-54081), (58504-58509), (60381-60386), (63240-63245), (64177-64182), |
| | (66825-66830), (67624-67629), (68110-68115), (70079-70084), (70297-70302), |
| | (70528-70533), (74805-74810), (75832-75837), (75929-75934), (79472-79477), |
| | (81732-81737), (86982-86987), (90396-90401), (93524-93529), (94074-94079) |
| T₆ | (669-674), (1110-111), (55573-5578), (8644-8649), (9586-9591), (16487-16492), |
| | (19561-19566), (21434-21439), (21943-21948), (22955-22960), (24521-24526), |
| | (24975-24980), (25879-25884), (30957-30962), (35536-35541), (36347-36352), |
| | (38044-38049), (44578-44583), (44987-44992), (48071-48076), (48863-48868), |
| | (50459-50463), (50719-50723), (52104-52109), (52951-52956), (55329-55334), |
| | (56138-56143), (56845-56850), (57236-57241), (57770-57775), (58001-58006), |
| | (58636-58641), (61907-61912), (62617-62622), (64036-64041), (64573-64578), |
| | (65005-65010), (62246-62252), (64165-64171), (66597-66602), (67580-67585), |
| | (68847-68852), (69763-69768), (69817-69822), (74606-74611), (82187-82192), |
| | (83886-83891), (83901-83906), (86759-86764), (93470-93475), (93477-93482), |
| | (93484-93489), (94442-94447) |
| G₆ | (19787-19792), (26954-26959), (35808-35813), (93226-93231) |
| C₆ | (18235-18240), (21871-21876), (76917-76922), (80991-80996), (88602-88607), |
| | (90791-90796) |
| A₇ | (1127-1133), (2538-2544), (10768-10774), (23606-23612), (23944-23950), |
| | (27773-27779), (34368-3434), (46357-46363), (57809-57815), (61118-61124), |
| | (38847-38853), (39086-39092), (44655-44661), (45892-45898), (49673-49679), |
| | (51719-51725), (56198-56204), (56273-56279), (56723-56729), (65739-65745), |
| | (72577-72583), (86233-86239), (88774-88780), (90171-90177), (90182-90188) |
| T₇ | (1640-1646), (3316-3322), (9361-9367), (13935-13941), (16902-16908), (19814- |
| | 19820), (36431-36437), (47567-47573), (47577-47583), (47591-47597), (51486- |
| | 51492), (52461-52467), (53076-53082), (60673-60679), (60938-60944), (61091- |
| | 61097), (61151-61157), (66753-66759), (69794-69800), (81809-81815), (85947- |
| | 85953), (86096-86102) |
| G₇ | (80396-80402) |
| C₇ | (80396-80402) |
| A₈ | (1528-1535), (1647-1654), (14549-14556), (21587-21594), (34357-34364), |
| | (38933-38940), (38959-38966), (50259-50266), (50517-50524), (51552-51559), |
| | (55070-55077), (59117-59124), (62107-62114), (89700-89707) |
| T₈ | (2868-2875), (4151-4158), (9530-9537), (10450-1045716114-16121), (28933- |
| | 28940), (32187-32194), (36439-36446), (55062-55069), (59070-59077), (65081- |
| | 65088), (66744-66751), (71525-71532), (80038-80045), (81713-81720), (86249- |
| | 86256) |
| A₉ | (2994-3002), (3895-3903), (34631-34639), (53835-53843), (60680-60688), |
| | (75758-75766), (80051-80059), (85924-85932), (94654-94662), (94956-94964) |
| T₉ | (44074-44082), (50314-50322), (51342-51350), (56146-56154), (59108-59116), |
| | (74183-74191) (93491-93499) |
| G₉ | (84729-84737) |
| A₁₀ | (9055-9064), (28807-28816), (40300-40309) (76450-76459), (81745-81754) |
| T₁₀ | (51538-51547), (93559-93568) |
| A₁₁ | (51460-51470) |
| T₁₁ | (65822-65832) |
| T₁₂ | (22989-23000), (65090-65101) |
| A₁₃ | (76426-76438) |
| T₁₃ | (81007-81019) |
| T₁₄ | (53061-53074) |
| A₁₅ | (59409-59423) |
| T₁₅ | (11183-11198) |
| A₁₉ | (46427-46445) |
| T₂₂ | (65866-65887) |
| G₂₃ | (44088-44111) |
| (AC)₄ | (5-12), (34823-34830) |
| (AT)₄ | (2858-2865), (7152-7159), (9245-9252), (9682-9689), ( (57495-57502), (79872- |
| | 79879), (94746-94753) |
| (AG)₄ | (10096-10103), (10105-10112), (26092-26099), (52051-52058), (65506-65513) |
| (CA)₄ | 59722-9729), (29256-29263), (38525-38532) |
| (GA)₄ | (10420-10427), (26422-26429), (58605-58612), (59939-59946), (65547-65554) |
| (CT)₄ | (3520-3527), (75688-75695), (90461-90468) |
| (TA)₄ | (50161-50168), (59147-59154), (61289-61296), (63215-63222) |
| (TC)₄ | (1077-1084), (1561-1568), (3512-3519), (49995-50002), (59395-59402), (69783- |
| | 69790) |
| (AT)₅ | (8352-8361), (59286-59295) |
| (AG)₅ | (6127-6136), (10084-10093) |
| (CT)₅ | (16425-16434) |
| (GA)₅ | (65525-65534) |
| (GA)₆ | (6114-6125) |
| (AG)₆ | (22612-22623) |
| (CT)₆ | (80190-80201) |
| (TA)₇ | (13627-13640) |
| (TG)₇ | (50080-50093) |
| (AC)₈ | (15-30) |
| (GA)₈ | (23323-23338), (74550-74565) |
| (CT)₈ | (90698-90713) |
| (AT)₁₁ | (50998-51019) |
| (CT)₁₂ | (81605-81628) |
| (AT)₁₃ | (12990-13015) |
| (AT)₁₄ | (5534-5561) |
| (CT)₂₀ | (50041-50080) |
| (GA)₂₃ | (6242-6286) |
| (TTC)₄ | (10466-10477) |
| (ATC)₄ | (30327-30338) |
| (AAG)₄ | (33347-33358) |
| (GAA)₄ | (38968-38979), (76440-76451) |
| (GGA)₇ | (33632-33652), (92104-92124) |
| (TTTG)₄ | (94678-94693) |
| (ATTT)₃ | (11387-11398) |
| (AATG)₃ | (21270-21281) |
| (GAAA)₃ | (38984-38995) |
| (CCTT)₃ | (52708-52719) |
| (GAAA)₃ | (54909-54920) |
| (AAAG)₃ | (82076-82087) |
| (TGTA)₅ | (57974-57993) |
| (ATATT)₃ | (14606-14620) |
| (AAAAT)₃ | (50521-50535) |
| (TTTAG)₃ | (63813-63827) |
| (TATAT)₁₆ | (14580-14661) |
| (GGGTC)₄ | (69893-69912) |
| (AAAAAT)₃ | (56200-56217), (56275-56292) |
| (CCTCTC)₄ | (5906-5929) |
| (GGGCCG)₃ | (82836-82853) |

Using the data provided above, those skilled in the art know how to test whether the SSRs identified are specific for the sex of the date palm.

### Example 3

### Validation of the Three Date Palm Sex-Specific Microsatellite Markers in Other Species of the Phoenix Genus

### Materials and Methods

DNA Extraction.

Each sample is composed of leaves which were lyophilized for 72 hours using the Alpha1-4LD Plus lyophilizer (Fisher Scientific, France). The lyophilized leaves were ground using the TissueLyser System (Qiagen, USA), and then the extraction was carried out using the Dneasy plant kit (Qiagen, USA) according to the manufacturer's protocol.

The DNA obtained was assayed with the Tecan GENios™ spectrofluorimeter (Tecan, Switzerland). The concentrations of all the samples were adjusted to 10 ng/μl for the rest of the manipulation.

Microsatellite Amplification by PCR.

The PCR reactions were carried out using an Eppendorf thermocycler (AG, Germany). A reaction volume of 20 μl was used, containing 10 ng of genomic DNA, 10× reaction buffer, 2 mM MgCl₂, 200 μM dNTPs, 1 U of polymerase, 10 pmol of fluorochrome-marked forward primer, 10 pmol of reverse primer, and 2 pmol, and MilliQ water. The microsatellite amplicons were analyzed using the ABI 3130XL genetic analyser (Applied BioSystems, USA). The allele sizes were identified using the GeneMapper software v3.7 (Applied BioSystems, Foster city, CA, USA).

### Results

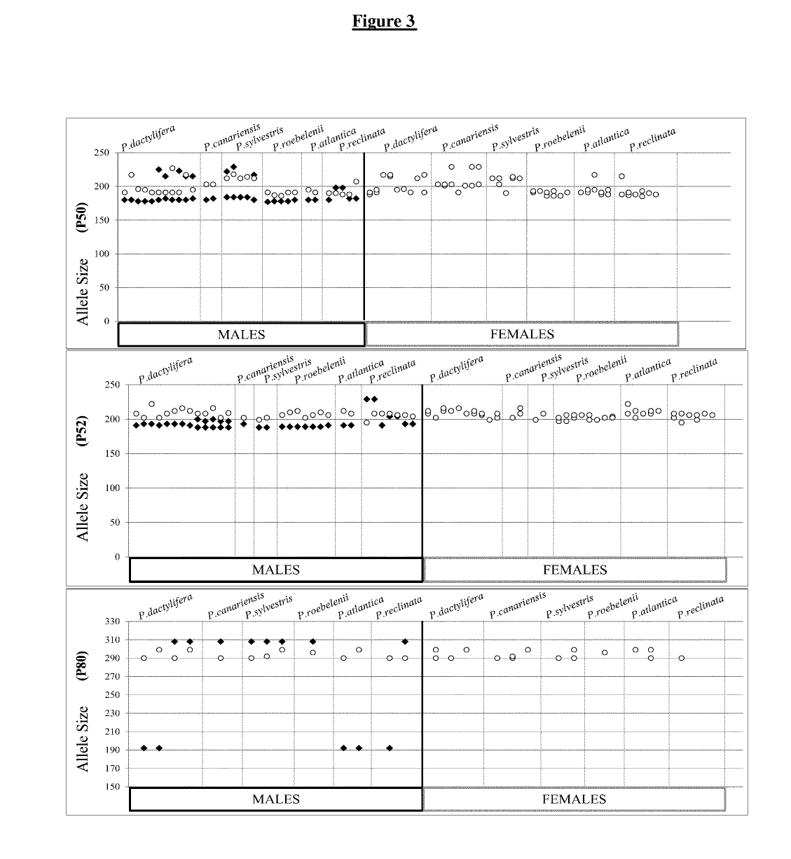

The results obtained are presented in FIG. 3. As shown in this figure, the three molecular markers P80, P50 and P52 also allow sex-determination in several other species of the Phoenix genus, in particular P. canariensis, P. sylvestris, P. roebelenii, P. atlatica and P. reclinata. This result reinforces the efficient ability of the inventive markers to discriminate between male and female date palm plants. It is highly probable that sex-linked regions are older than speciation within the Phoenix genus. This suggests that the three inventive markers are capable of sex discrimination in all individuals of the Phoenix genus, with the exception of very rare particular cases wherein the regions of interest have undergone slight variations.

### Example 4

### Identification of a Male-Specific Nucleotide Sequence

### Materials and Methods

DNA Extraction.

Leaf samples were freeze-dried for 72 hours with an Alpha1-4LD Plus lyophilizer (Fisher Scientific, Illkirch, France) and ground with a Tissue Lyser System (Qiagen). DNA extraction was carried out using the Dneasy plant mini kit (Qiagen) according to the manufacturer's instructions. The DNA was quantified with a Tecan GENios fluorescence microplate reader (Tecan, Mannedorf, Switzerland). All samples were adjusted to a concentration of 10 ng μl⁻¹ for subsequent analyses.

Genetic Analyses.

Polymerase chain reactions were performed in an Eppendorf (AG, Hamburg, Germany) thermocycler. The reaction volume was 20 μl and contained 10 ng of genomic DNA, 10× reaction buffer, 2 mM MgCl₂, 200 μM dNTPs, 0.5 U polymerase, 2 pmol of the forward primer is male-specific MYBF5 (SEQ ID NO: 19: TTCTCAGCCCCTCAAACTTC), 2 pmol of the reverse primer MYBR2 (SEQ ID NO: 20: GGTTGCAGCCATGAGCTCAACC), and MilliQ water. PCR was carried out with following parameters: denaturation for 2 min at 90° C., followed by 30 cycles of 90° C. for 30 s, 57° C. for 30 s and 72° C. for 1.5 min, and a final elongation step at 72° C. for 10 min.

### Results

While studying the evolution of sex-linked regions of the date palm genome, the present inventors have identified, in a region close to P80, an 18 nucleotide long sequence that is only present in male individuals. This male-specific sequence is located at position 74489 in scaffold PDK_{—}30s6550963 (GenBank accession number: GL739764.1) and has the sequence set forth in SEQ ID NO: 18 (AAGTTTGAGGGGCTGAGA).

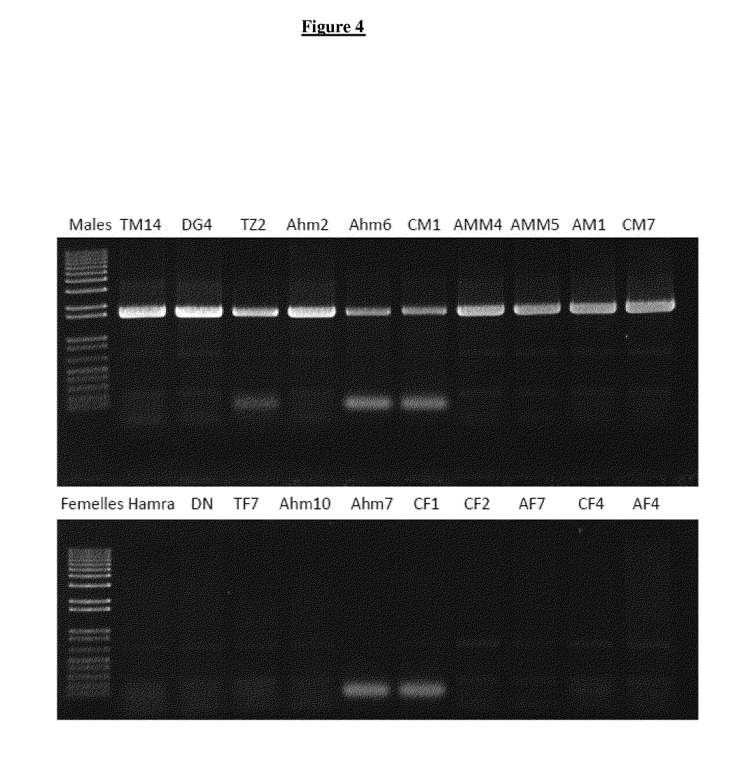

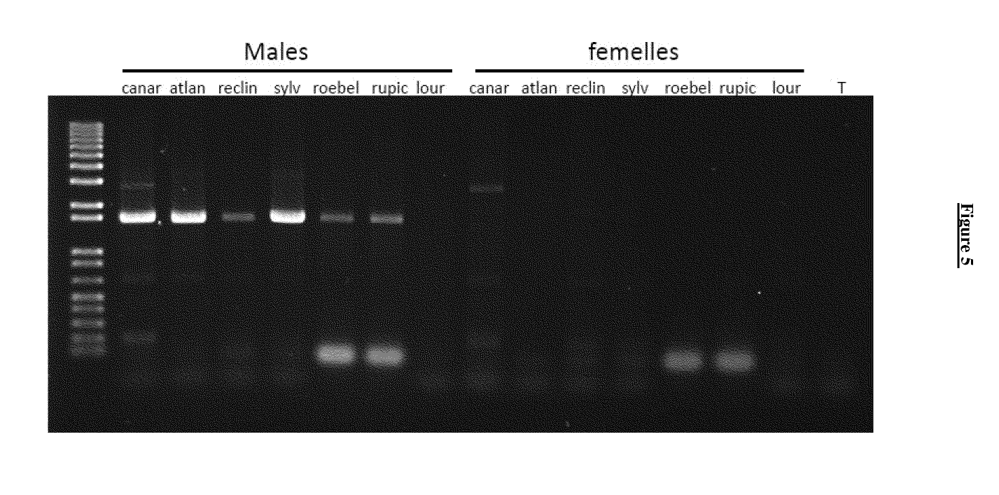

The male-specificity of this sequence has been verified on 10 males and 10 females of the P. dactylifera species (see FIG. 4). This sequence was also found to be male-specific in other species of the Phoenix genus, in particular in P. canariensis, P. sylvestris, P. roebellenii, P. atlantica, P. reclinata and P. rupicola (see FIG. 5).

1. A method for identifying the sex of a date palm by detection, using a microsatellite marker, of an SSR polymorphism in the date palm genome, the method comprising steps of:
amplifying a portion of genomic DNA of the date palm tested using a pair of primers specific of the microsatellite marker in order to obtain amplicons, and
analyzing the amplicons obtained in order to determine the sex of the date palm tested,
wherein the microsatellite marker is selected from the group consisting of:
the P80 microsatellite marker which, in the 5′→3′ direction, has a (ga) motif and is flanked, in 5′, by the nucleotide sequence occupying positions 73550 to 74549 in the PDK_{—}30s6550963 scaffold (GenBank accession number: GL739764.1), and in 3′, by the nucleotide sequence occupying positions 74566 to 75565 in the PDK_{—}30s6550963 scaffold;
the P50 microsatellite marker which, in the 5′→3′ direction, has an (ag) motif and is flanked, in 5′, by the nucleotide sequence occupying positions 4871 to 5870 in the PDK_{—}30s1202771 scaffold (GenBank accession number: GL744456.1), and in 3′, by the nucleotide sequence occupying positions 5891 to 6889 in the PDK_{—}30s1202771 scaffold; and
the P52 microsatellite marker which, in the 5′→3′ direction, has a (ct) motif and is flanked, in 5′, by the nucleotide sequence occupying positions 9276 to 10275 in the PDK_{—}30s680001 scaffold (GenBank accession number: GL745189.1), and in 3′, by the nucleotide sequence occupying positions 10308 to 11307 in the PDK_{—}30 s680001 scaffold.

2. The method according to claim 1, wherein the pair of primers specific of the microsatellite marker comprises:
if the microsatellite marker is P80: a forward SSR primer comprising, or consisting of, between 15 and 50 consecutive nucleotides of the sequence of at most 1000 nucleotides flanking, in 5′, position 74550 in the PDK_{—}30 s6550963 scaffold, and a reverse SSR primer comprising, or consisting of, between 15 and 50 consecutive nucleotides of the sequence complementary to the sequence of at most 1000 nucleotides flanking, in 3′, position 74565 in the PDK_{—}30s6550963 scaffold; or
if the microsatellite marker is P50: a forward SSR primer comprising, or consisting of, between 15 and 50 consecutive nucleotides of the sequence of at most 1000 nucleotides flanking, in 5′, position 5871 in the PDK_{—}30 s1202771 scaffold (GenBank accession number: GL744456.1), and a reverse SSR primer comprising, or consisting of, between 15 and 50 consecutive nucleotides of the sequence complementary to the sequence of at most 1000 nucleotides flanking, in 3′, position 5890 in the PDK_{—}30 s1202771 scaffold; or
if the microsatellite marker is P52: a forward SSR primer comprising, or consisting of, between 15 and 50 consecutive nucleotides of the sequence of at most 1000 nucleotides flanking, in 5′, position 10276 in the PDK_{—}30 s680001 scaffold, and a reverse SSR primer comprising, or consisting of, between 20 and 35 consecutive nucleotides of the sequence complementary to the sequence of at most 1000 nucleotides flanking, in 3′, position 10307 in the PDK_{—}30 s680001 scaffold.

3. The method according to claim 2, wherein the pair of primers specific for the microsatellite marker consists of:
if the microsatellite marker is P80: a forward SSR primer comprising, or consisting of, the sequence set forth in SEQ ID NO: 4 and a reverse SSR primer comprising, or consisting of, the sequence set forth in SEQ ID NO: 5; or
if the microsatellite marker is P50: a forward SSR primer comprising, or consisting of, the sequence set forth in SEQ ID NO: 6 and a reverse SSR primer comprising, or consisting of, the sequence set forth in SEQ ID NO: 7; or
if the microsatellite marker is P52: a forward SSR primer comprising, or consisting of, the sequence set forth in SEQ ID NO: 8 and a reverse SSR primer comprising, or consisting of, the sequence set forth in SEQ ID NO: 9.

4. The method according to claim 1, wherein analyzing the amplicons obtained in order to determine the sex of the date palm tested comprises steps of:
separating, according to their size, the amplicons obtained by amplification of the portion of the date palm genomic DNA; and
comparing the sizes of said amplicons with the sizes of the amplicons obtained by amplification, under the same conditions, of a portion of genomic DNA of a control date palm.

5. The method according to claim 4, wherein:
if the amplification is carried out with a pair of primers consisting of a forward SSR primer comprising, or consisting of, the sequence set forth in SEQ ID NO: 4 and a reverse SSR primer comprising, or consisting of, the sequence set forth in SEQ ID NO: 5, the presence of an amplicon containing 194 nucleotides and/or of an amplicon containing 310 nucleotides indicates that the date palm tested is a male palm plant;
if the amplification is carried out with a pair of primers consisting of a forward SSR primer comprising, or consisting of, the sequence set forth in SEQ ID NO: 6 and a reverse SSR primer comprising, or consisting of, the sequence set forth in SEQ ID NO: 7, the presence of an amplicon containing 180, 182, 214, 223, 225 or 227 nucleotides indicates that the date palm tested is a male palm plant;
if the amplification is carried out with a pair of primers consisting of a forward SSR primer comprising, or consisting of, the sequence set forth in SEQ ID NO: 8 and a reverse SSR primer comprising, or consisting of, the sequence set forth in SEQ ID NO: 9, the presence of an amplicon containing 188, 190, 191, 193, 197 or 199 nucleotides indicates that the date palm tested is a male palm plant.

6. The method according to claim 1, wherein analyzing the amplicons obtained in order to determine the sex of the date palm tested comprises a step of:
sequencing the amplicons in order to determine the number of repeats of the SSR motif of each amplicon.

7. The method according to claim 6, wherein analyzing the amplicons obtained in order to determine the sex of the date palm tested further comprises, prior to the sequencing step, a step of:
separating, according to their size, the amplicons obtained by amplification of the portion of the date palm genomic DNA.

8. The method according to claim 1, wherein the method is carried out on genomic DNA extracted from a sample of the date palm and wherein the date palm sample is a sample of protoplast, of callus, of embryos, of leaf, of trunk, of root, of offshoots of cutting of the date palm or any combination thereof.

9. The method according to claim 8, wherein the date palm sample is collected from a date palm plant up to 6 to 8 years after sowing.

10. A primer pair for the identification of the sex of a date palm consisting of:
a forward SSR primer comprising, or consisting of, between 15 and 50 consecutive nucleotides of the sequence of at most 1000 nucleotides flanking, in 5′, position 74550 in the PDK_{—}30 s6550963 scaffold, and a reverse SSR primer comprising, or consisting of, between 15 and 50 consecutive nucleotides of the sequence complementary to the sequence of at most 1000 nucleotides flanking, in 3′, position 74565 in the PDK_{—}30 s6550963 scaffold; or
a forward SSR primer comprising, or consisting of, between 15 and 50 consecutive nucleotides of the sequence of at most 1000 nucleotides flanking, in 5′, position 5871 in the PDK_{—}30 s1202771 scaffold (GenBank accession number: GL744456.1), and a reverse SSR primer comprising, or consisting of, between 15 and 50 consecutive nucleotides of the sequence complementary to the sequence of at most 1000 nucleotides flanking, in 3′, position 5890 in the PDK_{—}30 s1202771 scaffold; or
a forward SSR primer comprising, or consisting of, between 15 and 50 consecutive nucleotides of the sequence of at most 1000 nucleotides flanking, in 5′, position 10276 in the PDK_{—}30 s680001 scaffold, and a reverse SSR primer comprising, or consisting of, between 20 and 35 consecutive nucleotides of the sequence complementary to the sequence of at most 1000 nucleotides flanking, in 3′, position 10307 in the PDK_{—}30 s680001 scaffold.

11. The primer pair according to claim 10 comprising:
a forward SSR primer comprising, or consisting of, the sequence set forth in SEQ ID NO: 4 and a reverse SSR primer comprising, or consisting of, the sequence set forth in SEQ ID NO: 5; or
a forward SSR primer comprising, or consisting of, the sequence set forth in SEQ ID NO: 6 and a reverse SSR primer comprising, or consisting of, the sequence set forth in SEQ ID NO: 7; or
a forward SSR primer comprising, or consisting of, the sequence set forth in SEQ ID NO: 8 and a reverse SSR primer comprising, or consisting of, the sequence set forth in SEQ ID NO: 9.

12. The primer pair according to claim 10, wherein at least one of the primers of the primer pair comprises a detectable label.

13. (canceled)

14. A kit for identifying the sex of a date palm comprising at least one primer pair according to claim 10, and instructions for identifying the sex of a date palm using a method according to claim 1.

15. (canceled)

16. A method for identifying the sex of a date palm comprising steps of:
amplifying a portion of genomic DNA of the date palm tested using a pair of primers specific of a male-specific marker in order to obtain amplicons, and
detecting if amplicons are produced, wherein detection of amplicons indicates that the date palm tested as a male date palm,
wherein, in the 5′→3′ direction, the male-specific marker is located at position 74489 in scaffold PDK_{—}30 s6550963 and has the sequence set forth in SEQ ID NO: 18.

17. The method according to claim 16, wherein the pair of primers specific for the male-specific marker consists of:
a forward PCR primer comprising, or consisting of, between 15 and 18 consecutive nucleotides of the sequence of at most 2000 nucleotides flanking, in 5′, position 74489 in the PDK_{—}30 s6550963 scaffold, and
a reverse PCR primer comprising, or consisting of, between 15 and 18 consecutive nucleotides of the sequence complementary to the sequence of at most 2000 nucleotides flanking, in 3′, position 74506 in the PDK_{—}30 s6550963 scaffold.

18. The method according to claim 17, wherein the forward PCR primer comprises, or consists of, the sequence set forth in SEQ ID NO: 19 and the reverse PCR primer comprises, or consists of, the sequence set forth in SEQ ID NO: 20.

19. The method according to claim 16, wherein the method is carried out on genomic DNA extracted from a sample of the date palm and wherein the date palm sample is a sample of protoplast, of callus, of embryos, of leaf, of trunk, of root, of offshoots of cutting of the date palm or any combination thereof.

20. The method according to claim 19, wherein the date palm sample is collected from a date palm plant up to 6 to 8 years after sowing.

21. A primer pair for the identification of the sex of a date palm consisting of:
a forward PCR primer comprising, or consisting of, between 15 and 18 consecutive nucleotides of the sequence of at most 2000 nucleotides flanking, in 5′, position 74489 in the PDK_{—}30 s6550963 scaffold, and
a reverse PCR primer comprising, or consisting of, between 15 and 18 consecutive nucleotides of the sequence complementary to the sequence of at most 2000 nucleotides flanking, in 3′, position 74506 in the PDK_{—}30 s6550963 scaffold.

22. The primer pair according to claim 21, wherein the forward PCR primer comprises, or consists of, the sequence set forth in SEQ ID NO: 19 and the reverse PCR primer comprises, or consists of, the sequence set forth in SEQ ID NO: 20.

23. The primer pair according to claim 21 or claim 22, wherein at least one of the primers of the primer pair comprises a detectable label.

24. (canceled)

25. A kit for identifying the sex of a date palm comprising at least one primer pair according to claim 21 and instructions to identify the sex of a date palm using a method according to claim 16.

26. (canceled)

27. (canceled)
